(19) United States
(12) Patent Application Publication
(10) Pub. No.: US 2009/0240055 A1
(43) Pub. Date: Sep. 24, 2009

(54) Method for 1H-Imidazo[4,5-C] Pyridines and Analogs Thereof

(76) Inventors: Larry R. Krepski, White Bear Lake, MN (US); Gregory J. Marszalek, Woodbury, MN (US); Sonja S. Mackey, St. Paul, MN (US); John F. Gerster, Woodbury, MN (US)

Correspondence Address: WOLF GREENFIELD & SACKS, P.C., 600 ATLANTIC AVENUE, BOSTON, MA, 02210-2206, US

(21) Appl. No.: 11/992,371

(22) PCT Filed: Sep. 22, 2006
(86) PCT No.: PCT/US2006/037317

§ 371 Date: May 6, 2009

Related U.S. Application Data
(60) Provisional application No. 60/720,171, filed on Sep. 23, 2005.
(60) Provisional application No. 60/743,505, filed on Mar. 16, 2006.

(51) Int. Cl. C07D 471/04

(52) U.S. Cl. 546/82

(57) ABSTRACT

Methods and intermediates for preparing compounds of the Formulas: (I and X) are disclosed. The methods include a method providing a compound of the Formula: (IV) and converting a compound of Formula IV to a compound of Formula I, a method providing a compound of the Formula: (VIII) and converting a compound of Formula VIII to a compound of Formula I, and a method providing a compound of the Formula: (XI) and converting a compound of Formula XI to a compound of Formula I.

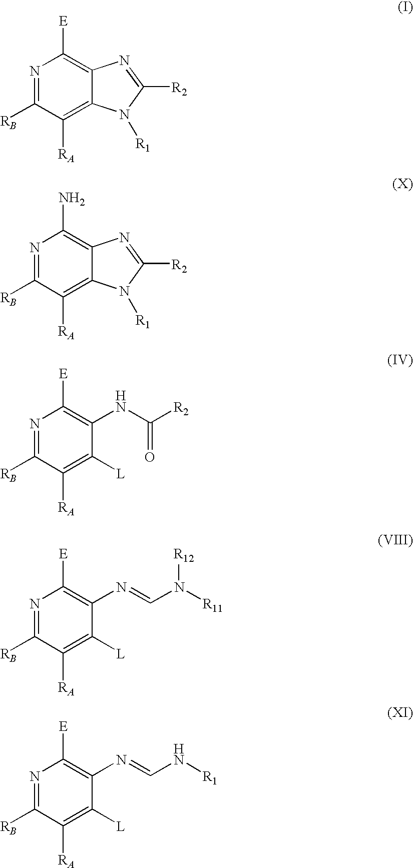

### CROSS-REFERENCE TO RELATED APPLICATIONS

This application claims priority to U.S. Provisional Application Ser. No. 60/720,171, filed on Sep. 23, 2005, and U.S. Provisional Application Ser. No. 60/743,505, filed on Mar. 16, 2006, both of which are incorporated herein by reference.

### BACKGROUND

Certain compounds have been found to be useful as immune response modifiers (IRMs), rendering them useful in the treatment of a variety of disorders. However, there continues to be interest in and a need for compounds that have the ability to modulate the immune response, by induction of cytokine biosynthesis or other mechanisms. Thus, there is a need for methods and intermediates for making such compounds.

### SUMMARY

It has now been found that certain 1H-imidazo[4,5-c]pyridines and analogs thereof, or pharmaceutically acceptable salts thereof, can be prepared by a method comprising:

providing a compound of the Formula IV:

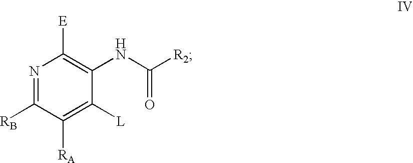

and reacting the compound of Formula IV with an amine of the formula R₁NH₂ to provide a 1H-imidazo[4,5-c]pyridine or analog thereof of the Formula I:

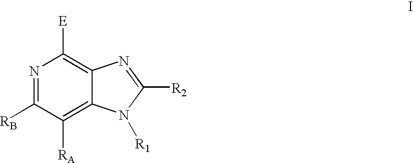

or a pharmaceutically acceptable salt thereof; wherein E, L, R₁, R₂, R_{A}, and R_{B} are defined below.

In another embodiment, certain 1H-imidazo[4,5-c]pyridines and analogs thereof, or pharmaceutically acceptable salts thereof, can be prepared by a method comprising:

providing a compound of the Formula VIII:

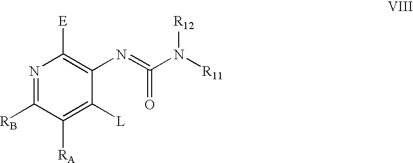

and reacting the compound of Formula VIII with an amine of the formula R₁NH₂ to provide a 1H-imidazo[4,5-c]pyridine or analog thereof of the Formula I:

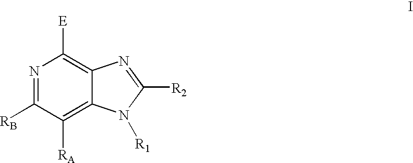

or a pharmaceutically acceptable salt thereof, wherein E, L, R₁, R₂, R₁₁, R₁₂, R_{A}, and R_{B} are defined below.

In another embodiment, certain 1H-imidazo[4,5-c]pyridines and analogs thereof, or pharmaceutically acceptable salts thereof, can be prepared by a method comprising:

providing a compound of the Formula XI:

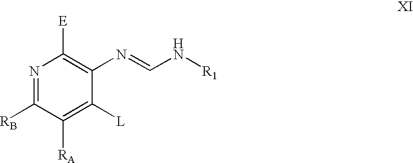

and forming a 1H-imidazo[4,5-c]pyridine or analog thereof of the Formula I:

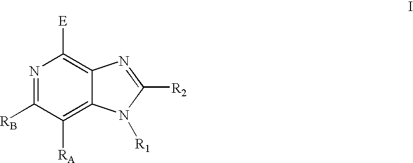

or a pharmaceutically acceptable salt thereof; wherein E, L, R₁, R₂, R_{A}, and R_{B} are defined below.

Compounds and salts of Formula I are useful for making immune response modifying compounds of the following Formula X:

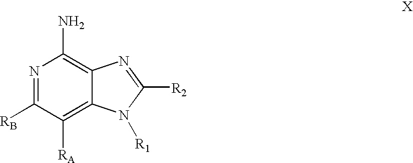

or pharmaceutically acceptable salts thereof; wherein R₁, R₂, R_{A}, and R_{B} are defined below. The compounds and salts of Formula X are known to be useful as immune response modifiers due to their ability to induce or inhibit cytokine biosynthesis (e.g., induces or inhibits the biosynthesis of at least one cytokine) and otherwise modulate the immune response when administered to animals. This makes these compounds and salts useful in the treatment of a variety of conditions such as viral diseases and tumors that are responsive to such changes in the immune response.

In one embodiment, there is provided a method that includes:

providing a compound of the Formula IV:

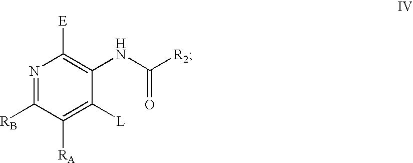

reacting the compound of Formula IV with an amine of the formula R₁NH₂ to provide a 1H-imidazo[4,5-c]pyridine or analog thereof of the Formula I:

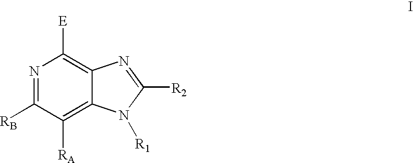

or a pharmaceutically acceptable salt thereof; and

converting E to an amino group in the compound of Formula I to provide a compound (a 1H-imidazo[4,5-c]pyridin-4-amine or analog thereof) of the Formula X:

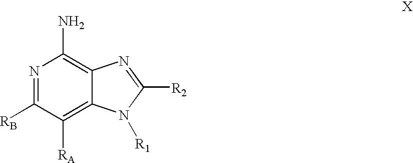

or a pharmaceutically acceptable salt thereof; wherein E, L, R₁, R₂, R_{A}, and R_{B} are defined below.

In another aspect, the invention provides intermediates useful in the preparation of immune response modifiers. In one embodiment, there is provided a compound of the Formula XI:

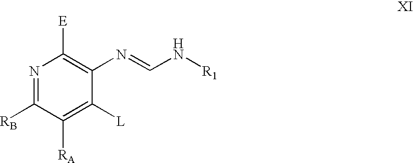

wherein E, L, R₁, R_{A}, and R_{B} are defined below.

As used herein, “a”, “an”, “the”, “at least one”, and “one or more” are used interchangeably.

The terms “comprises” and variations thereof do not have a limiting meaning where these terms appear in the description and claims.

The above summary of the present invention is not intended to describe each disclosed embodiment or every implementation of the present invention. The description that follows more particularly exemplifies illustrative embodiments. In several places throughout the description, guidance is provided through lists of examples, which examples can be used in various combinations. In each instance, the recited list serves only as a representative group and should not be interpreted as an exclusive list.

### DETAILED DESCRIPTION OF ILLUSTRATIVE EMBODIMENTS OF THE INVENTION

The present invention provides methods and intermediates for preparing certain 1H-imidazo[4,5-c]pyridines and analogs thereof of the Formula I:

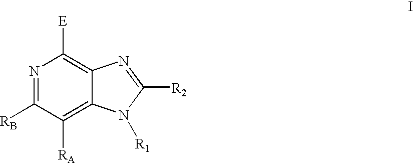

or pharmaceutically acceptable salts thereof; which are useful for preparing compounds (1H-imidazo[4,5-c]pyridin-4-amines or analog thereof) of the Formula X:

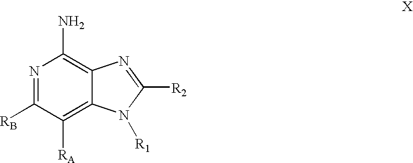

or pharmaceutically acceptable salts thereof, wherein E, R₁, R₂, R_{A}, and R_{B} are defined below.

In one embodiment, there is provided a method (i) comprising:

providing a compound of the Formula IV:

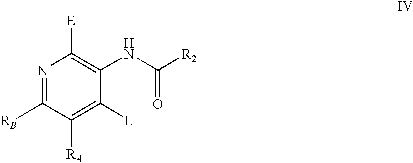

and reacting the compound of Formula IV with an amine of the formula R₁NH₂ to provide a 1H-imidazo[4,5-c]pyridine or analog thereof of the Formula I:

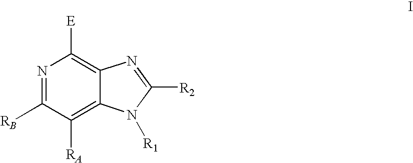

or a pharmaceutically acceptable salt thereof;
wherein:

E is selected from the group consisting of hydrogen, fluoro, chloro, bromo, iodo, hydroxy, phenoxy, —O—S(O)₂—R′, and —N(Bn)₂, wherein R′ is selected from the group consisting of alkyl, haloalkyl, and aryl optionally substituted by alkyl, halo, or nitro, and Bn is selected from the group consisting of benzyl, p-methoxybenzyl, p-methylbenzyl, and 2-furanylmethyl; or

E is joined with the adjacent pyridine nitrogen atom of Formulas I and IV to form the fused tetrazolo ring in Formulas I-1 and IV-1:

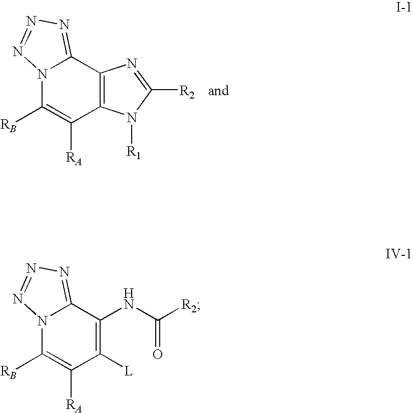

L is selected from the group consisting of fluoro, chloro, bromo, iodo, phenoxy, and —O—S(O)₂—R′, wherein R′ is selected from the group consisting of alkyl, haloalkyl, and aryl optionally substituted by alkyl, halo, or nitro;

R_{A} and R_{B} are independently selected from the group consisting of:
hydrogen,
halogen,
alkyl,
alkenyl,
alkoxy,
alkylthio, and
—N(R₉)₂;

or R_{A} and R_{B} taken together form a fused benzene ring or a fused pyridine ring wherein the benzene ring or pyridine ring is unsubstituted or substituted by one R group, or substituted by one R₃ group, or substituted by one R group and one R₃ group;

or R_{A} and R_{B} taken together form a fused 5 to 7 membered saturated ring optionally containing one nitrogen atom, wherein the fused ring is unsubstituted or substituted by one or more R groups;

R is selected from the group consisting of:
halogen,
hydroxy,
alkyl,
alkenyl,
haloalkyl,
alkoxy,
alkylthio, and
—N(R₉)₂;

R₁ is selected from the group consisting of:
-R₄,
-X-R₄,
-X-Y-R₄,
-X-Y-X-Y-R₄,
-X-R₅,
—N(R₁′)-Q-R₄,
—N(R₁′)—X₁—Y₁—R₄, and
—N(R₁′)—X₁—R_{5b};

R₂ is selected from the group consisting of:
-R₄,
-X-R₄,
-X-Y-R₄, and
-X-R₅;

R₃ is selected from the group consisting of:
-Z-R₄,
-Z-X-R₄,
-Z-X-Y-R₄,
-Z-X-Y-X-Y-R₄, and
-Z-X-R₅;

X is selected from the group consisting of alkylene, alkenylene, alkynylene, arylene, heteroarylene, and heterocyclylene wherein the alkylene, alkenylene, and alkynylene groups can be optionally interrupted or terminated by arylene, heteroarylene or heterocyclylene and optionally interrupted by one or more —O— groups;

X₁ is C₂₋₂₀ alkylene;

Y is selected from the group consisting of:
—O—,
—S(O)₀₋₂—,
—S(O)₂—N(R₈)—,
—C(R₆)—,
—O—C(R₆)—,
—O—C(O)—O—,
—N(R₈)-Q-,
—O—C(R₆)—N(R₈)—,
—C(R₆)—N(OR₉)—,
—O—N(R₈)-Q-,
—O—N═C(R₄)—,
—C(═N—O—R₈)—,
—CH(—N(—O—R₈)-Q-R₄)—,

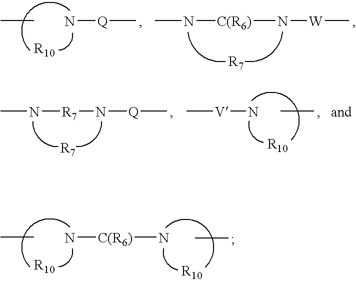

Y₁ is selected from the group consisting of —O—, —S(O)₀₋₂—, —S(O)₂—N(R₈)—, —N(R₈)-Q-, —C(R₆)—N(R₈)—, —O—C(R₆)—N(R₈)—, and

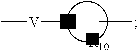

Z is a bond or —O—;

R₁′ is selected from the group consisting of hydrogen, C₁₋₂₀ alkyl, hydroxy-C₂₋₂₀ alkylenyl, and all-oxy-C₂₋₂₀ alkylenyl;

R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of allyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;

R₅ is selected from the group consisting of:

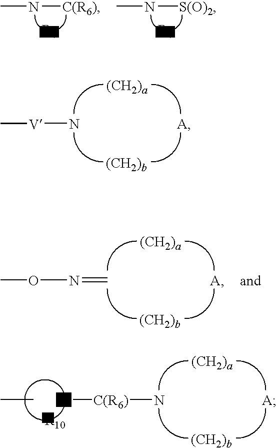

R_{5b} is selected from the group consisting of:

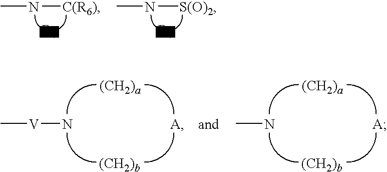

R₆ is selected from the group consisting of ═O and ═S;

R₇ is C₂₋₇ alkylene;

R₈ is selected from the group consisting of hydrogen, alkyl, alkoxyalkylenyl, hydroxyalkylenyl, arylalkylenyl, and heteroarylalkylenyl;

R₉ is selected from the group consisting of hydrogen and alkyl;

R₁₀ is C₃₋₈ allylene;

A is selected from the group consisting of —O—, —C(O)—, —S(O)₀₋₂—, and —N(R₄)—;

A′ is selected from the group consisting of —O—, —S(O)₀₋₂—, —N(-Q-R₄)—, and —CH₂—;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, —C(R₆)—S—, and —C(R₆)—N(OR₉)—;

V is selected from the group consisting of —C(R₆)—, —O—C(R₆)—, —N(R₈)—C(R₆)—, and —S(O)₂—;

V′ is selected from the group consisting of —O—C(R₆)—, —N(R₈)—C(R₆)—, and —S(O)₂—;

W is selected from the group consisting of a bond, —C(O)—, and —S(O)₂—; and

a and b are independently integers from 1 to 6 with the proviso that a+b is ≦7.

This ring forming reaction is unexpected, since the L group is displaced without a strong electron withdrawing group adjacent the L group.

In another embodiment, there is provided a method (ii) wherein the above method (i) further comprises the steps of:

providing a compound of the Formula III:

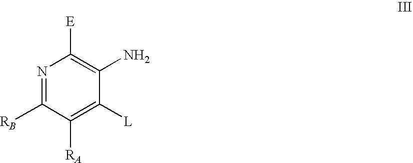

and reacting the compound of Formula III with a carboxylic acid halide of the formula hal-C(O)—R₂, wherein hal is chloro or bromo, or an anhydride or mixed anhydride of the formula O(—C(O)—R₂)₂ to provide an compound of Formula IV.

In another embodiment, there is provided a method (iii) wherein the above method (ii) further comprises the steps of:

providing a compound of the Formula II:

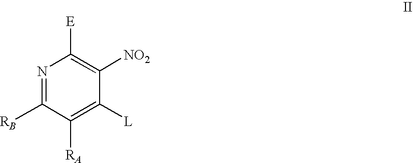

and reducing the compound of Formula II to provide a compound of Formula III.

In another embodiment, there is provided a method (iv) wherein the method (ii) further comprises the steps of:

providing a compound of the Formula VI:

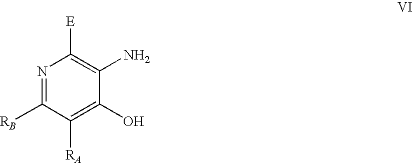

and converting the hydroxy group at the 4-position of Formula VI to an L group to provide a compound of Formula III.

In another embodiment, there is provided a method (v) wherein the method (i) further comprises the steps of:

providing a compound of the Formula VII:

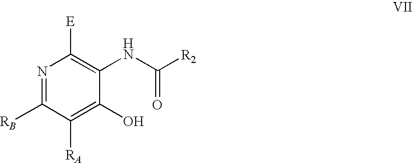

and converting the hydroxy group at the 4-position of Formula VII to an L group to provide a compound of Formula IV.

In another embodiment, there is provided a method (vi) wherein the method (v) further comprises the steps of:

providing a compound of the Formula VI:

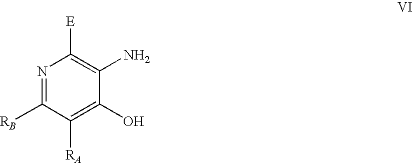

and reacting the compound of Formula VI with a carboxylic acid halide of the formula hal-C(O)—R₂, wherein hal is a chloro or bromo, or an anhydride or mixed anhydride of the formula O(—C(O)—R₂)₂ to provide an compound of Formula VII.

In other embodiments, there is provided a method (vii) or (viii) wherein the method (iv) or (vi), respectively, further comprises the steps of:

providing a compound of the Formula V:

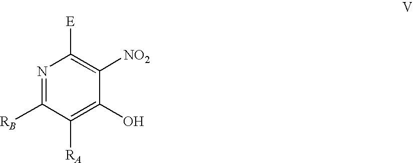

and reducing the compound of Formula V to provide a compound of Formula VI.

In one embodiment, there is provided a method (ix) that includes:

providing a compound of the Formula VIII:

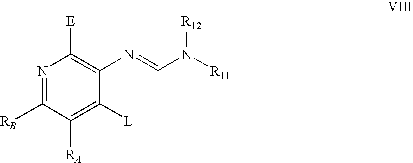

and reacting the compound of Formula VIII with an amine of the formula R₁NH₂ to provide a 1H-imidazo[4,5-c]pyridine or analog thereof of the Formula I:

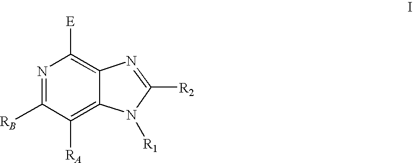

or a pharmaceutically acceptable salt thereof;
wherein:

E is selected from the group consisting of hydrogen, fluoro, chloro, bromo, iodo, hydroxy, phenoxy, —O—S(O)₂—R′, and —N(Bn)₂, wherein R′ is selected from the group consisting of alkyl, haloalkyl, and aryl optionally substituted by alkyl, halo, or nitro, and Bn is selected from the group consisting of benzyl, p-methoxybenzyl, p-methylbenzyl, and 2-furanylmethyl; or

E is joined with the adjacent pyridine nitrogen atom of Formulas I and VIII to form the fused tetrazolo ring in Formulas I-1 and IX:

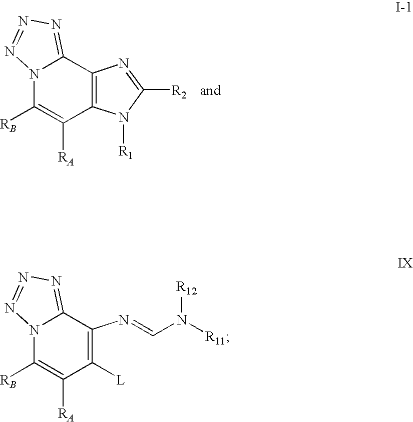

L is selected from the group consisting of fluoro, chloro, bromo, iodo, phenoxy, and —O—S(O)₂—R′, wherein R′ is selected from the group consisting of alkyl, haloalkyl, and aryl optionally substituted by alkyl, halo, or nitro;

R_{A} and R_{B} are independently selected from the group consisting of:
hydrogen,
halogen,
alkyl,
alkenyl,
alkoxy,
alkylthio, and
—N(R₉)₂;

or R_{A} and R_{B} taken together form a fused benzene ring or a fused pyridine ring wherein the benzene ring or pyridine ring is unsubstituted or substituted by one R group, or substituted by one R₃ group, or substituted by one R group and one R₃ group;

or R_{A} and R_{B} taken together form a fused 5 to 7 membered saturated ring optionally containing one nitrogen atom, wherein the fused ring is unsubstituted or substituted by one or more R groups;

R is selected from the group consisting of:
halogen,
hydroxy,
alkyl,
alkenyl,
haloalkyl,
alkoxy,
alkylthio, and
—N(R₉)₂;

R₁ is selected from the group consisting of:
-R₄,
-X-R₄,
-X-Y-R₄,
-X-Y-X-Y-R₄,
-X-R₅,
-N(R₁′)-Q-R₄,
—N(R₁′)—X₁—Y₁—R₄, and
—N(R₁′)—X₁—R_{5b};

R₂ is hydrogen;

R₃ is selected from the group consisting of:
-Z-R₄,
-Z-X-R₄,
-Z-X-Y-R₄,
-Z-X-Y-X-Y-R₄, and
-Z-X-R₅;

X is selected from the group consisting of allylene, alkenylene, alkynylene, arylene, heteroarylene, and heterocyclylene wherein the alkylene, alkenylene, and alkynylene groups can be optionally interrupted or terminated by arylene, heteroarylene or heterocyclylene and optionally interrupted by one or more —O— groups;

X₁ is C₂₋₂₀ alkylene;

Y is selected from the group consisting of:
—O—,
—S(O)₀₋₂—,
—S(O)₂—N(R₈)—,
—C(R₆)—,
—O—C(R₆)—,
—O—C(O)—O—,
—N(R₈)-Q-,
—O—C(R₆)—N(R₈)—,
—C(R₆)—N(OR₉)—,
—O—N(R₈)-Q-,
—O—N═C(R₄)—,
—C(═N—O—R₈)—,
—CH(—N(—O—R₈)-Q-R₄)—,

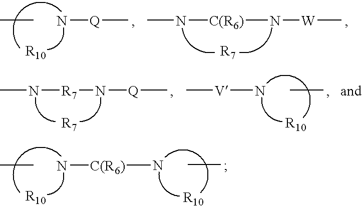

Y₁ is selected from the group consisting of —O—, —S(O)₀₋₂—, —S(O)₂—N(R₈)—, —N(R₈)-Q-, —C(R₆)—N(R₈)—, —O—C(R₆)—N(R₈)—, and

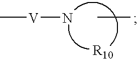

Z is a bond or —O—;

R₁′ is selected from the group consisting of hydrogen, C₁₋₂₀ alkyl, hydroxy-C₂₋₂₀ alkylenyl, and alkoxy-C₂₋₂₀ alkylenyl;

R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;

R₅ is selected from the group consisting of:

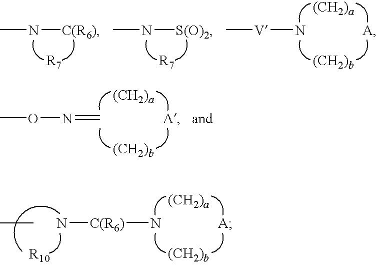

R_{5b} is selected from the group consisting of:

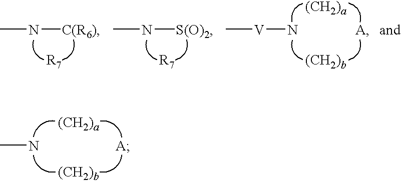

R₆ is selected from the group consisting of ═O and ═S;

R₇ is C₂₋₇ alkylene;

R₈ is selected from the group consisting of hydrogen, alkyl, alkoxyalkylenyl, hydroxyalkylenyl, arylalkylenyl, and heteroarylalkylenyl;

R₉ is selected from the group consisting of hydrogen and alkyl;

R₁₀ is C₃₋₈ alkylene;

R₁₁ and R₁₂ are independently C₁₋₄ allyl or R₁₁ and R₁₂ together with the nitrogen atom to which they are attached form a 5- or 6-membered ring optionally containing —O—, —N(C₁₋₄ alkyl)-, or —S—;

A is selected from the group consisting of —O—, —C(O)—, —S(O)₀₋₂—, and —N(R₄)—;

A′ is selected from the group consisting of —O—, —S(O)₀₋₂—, —N(-Q-R₄)—, and —CH₂—;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, —C(R₆)—S—, and —C(R₆)—N(OR₉)—;

V is selected from the group consisting of —C(R₆)—, —O—C(R₆)—, —N(R₈)—C(R₆)—, and —S(O)₂—;

V′ is selected from the group consisting of —O—C(R₆)—, —N(R₈)—C(R₆)—, and —S(O)₂—;

W is selected from the group consisting of a bond, —C(O)—, and —S(O)₂—; and

a and b are independently integers from 1 to 6 with the proviso that a+b is ≦7.

This ring forming reaction is also unexpected, since the L group is displaced without a strong electron withdrawing group adjacent the L group.

In another embodiment, there is provided a method (x) wherein the above method (ix) further comprises:

forming an intermediate of Formula XI:

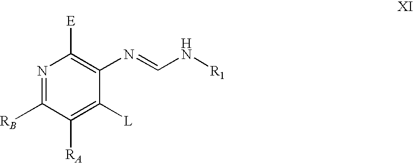

after reacting the compound of Formula VIII with an amine of the formula R₁NH₂.

In another embodiment, there is provided a method (xi) wherein the intermediate of Formula XI in method (x) above is isolated after reacting the compound of Formula VIII with an amine of the formula R₁NH₂.

In other embodiments, there is provided a method (xii) or (xiii) wherein the above method (ix) or (x), respectively, further comprises:

providing a compound of the Formula VI:

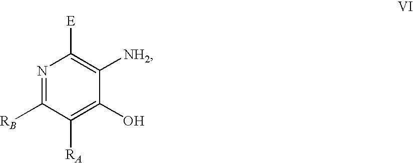

converting the hydroxy group at the 4-position to an L group,
and reacting the amino group at the 3-position with a formamide of the formula H—C(O)—N(R₁₁)R₁₂ to provide a compound of Formula VIII.

In other embodiments, there is provided a method (xiv) or (xv) wherein the compound of Formula VIII in the above method (xii) or (xiii), respectively, is provided without being isolated prior to reacting with an amine of the formula R₁NH₂.

In one embodiment, there is provided a method (xvi) that includes:

providing a compound of the Formula XI:

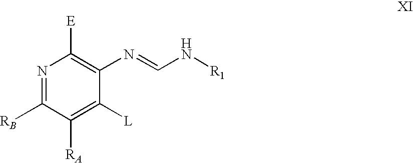

and forming a 1H-imidazo[4,5-c]pyridine or analog thereof of the Formula I:

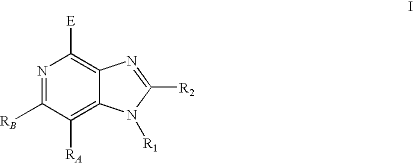

or a pharmaceutically acceptable salt thereof;
wherein:

E is selected from the group consisting of hydrogen, fluoro, chloro, bromo, iodo, hydroxy, phenoxy, —O—S(O)₂—R′, and —N(Bn)₂, wherein R′ is selected from the group consisting of alkyl, haloalkyl, and aryl optionally substituted by alkyl, halo, or nitro, and Bn is selected from the group consisting of benzyl, p-methoxybenzyl, p-methylbenzyl, and 2-furanylmethyl; or

E is joined with the adjacent pyridine nitrogen atom of Formulas I and XI to form the fused tetrazolo ring in Formulas I-1 and XIII:

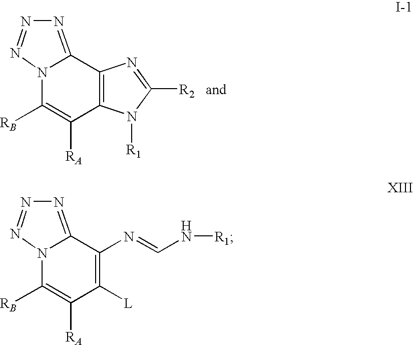

L is selected from the group consisting of fluoro, chloro, bromo, iodo, phenoxy, and —O—S(O)₂—R′, wherein R′ is selected from the group consisting of alkyl, haloalkyl, and aryl optionally substituted by alkyl, halo, or nitro;

R_{A} and R_{B} are independently selected from the group consisting of:
hydrogen,
halogen,
alkyl,
alkenyl,
alkoxy,
alkylthio, and
—N(R₉)₂;

or R_{A} and R_{B} taken together form a fused benzene ring or a fused pyridine ring wherein the benzene ring or pyridine ring is unsubstituted or substituted by one R group, or substituted by one R₃ group, or substituted by one R group and one R₃ group;

or R_{A} and R_{B} taken together form a fused 5 to 7 membered saturated ring optionally containing one nitrogen atom, wherein the fused ring is unsubstituted or substituted by one or more R groups;

R is selected from the group consisting of:
halogen,
hydroxy,
alkyl,
alkenyl,
haloalkyl,
alkoxy,
alkylthio, and
—N(R₉)₂;

R₁ is selected from the group consisting of:
-R₄,
-X-R₄,
-X-Y-R₄,
-X-Y-X-Y-R₄,
-X-R₅,
N(R₁′)-Q-R₄,
—N(R₁′)—X₁—Y₁—R₄, and
—N(R₁′)—X₁—R_{5b};

R₂ is hydrogen;

R₃ is selected from the group consisting of:
-Z-R₄,
-Z-X-R₄,
-Z-X-Y-R₄,
-Z-X-Y-X-Y-R₄, and
-Z-X-R₅;

X is selected from the group consisting of alkylene, alkenylene, alkynylene, arylene, heteroarylene, and heterocyclylene wherein the alkylene, alkenylene, and alkynylene groups can be optionally interrupted or terminated by arylene, heteroarylene or heterocyclylene and optionally interrupted by one or more —O— groups;

X₁ is C₂₋₂₀ alkylene;

Y is selected from the group consisting of:
—O—,
—S(O)₀₋₂—,
—S(O)₂—N(R₈)—,
—C(R₆)—,
—O—C(R₆)—,
—O—C(O)—O—,
—N(R₈)-Q-,
—O—C(R₆)—N(R₈)—,
—C(R₆)—N(OR₉)—,
—O—N(R₈)-Q-,
—O—N═C(R₄)—,
—C(═N—O—R₈)—,
—CH(—N(—O—R₈)-Q-R₄)—,

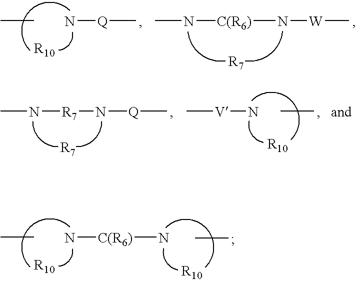

Y₁ is selected from the group consisting of —O—, —S(O)₀₋₂—, —S(O)₂—N(R₈)—, —N(R₈)-Q-, —C(R₆)—N(R₈)—, —O—C(R₆)—N(R₈)—, and

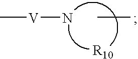

Z is a bond or —O—;

R₁′ is selected from the group consisting of hydrogen, C₁₋₂₀ alkyl, hydroxy-C₂₋₂₀ alkylenyl, and alkoxy-C₂₋₂₀ alkylenyl;

R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the allyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;

R₅ is selected from the group consisting of:

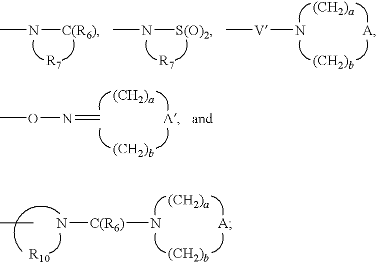

R_{5b} is selected from the group consisting of:

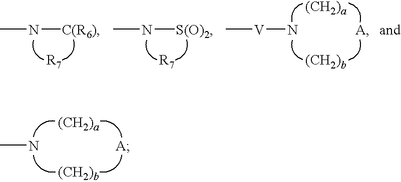

R₆ is selected from the group consisting of ═O and ═S;

R₇ is C₂₋₇ allylene;

R₈ is selected from the group consisting of hydrogen, alkyl, alkoxyalkylenyl, hydroxyalkylenyl, arylalkylenyl, and heteroarylalkylenyl;

R₉ is selected from the group consisting of hydrogen and alkyl;

R₁₀ is C₃₋₈ alkylene;

A is selected from the group consisting of —O—, —C(O)—, —S(O)₀₋₂—, and —N(R₄)—;

A′ is selected from the group consisting of —O—, —S(O)₀₋₂—, —N(-Q-R₄)—, and —CH₂—;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, —C(R₆)—S—, and —C(R₆)—N(OR₉)—;

V is selected from the group consisting of —C(R₆)—, —O—C(R₆)—, —N(R₈)—C(R₆)—, and —S(O)₂—;

V′ is selected from the group consisting of —O—C(R₆)—, —N(R₈)—C(R₆)—, and —S(O)₂—;

W is selected from the group consisting of a bond, —C(O)—, and —S(O)₂—; and

a and b are independently integers from 1 to 6 with the proviso that a+b is ≦7.

This ring forming reaction is also unexpected, since the L group is displaced without a strong electron withdrawing group adjacent the L group.

In another embodiment, there is provided a method (xvii) wherein the above method (xvi) further comprises:

providing a compound of the Formula VI:

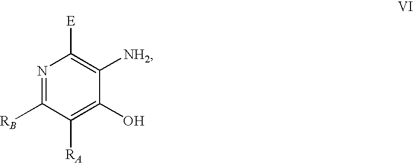

converting the hydroxy group at the 4-position to an L group,
and reacting the amino group at the 3-position with a formamide of the formula H—C(O)—NH(R₁) to provide a compound of Formula XI.

In another embodiment, there is provided a method (xviii) wherein the compound of Formula XI in the above method (xvii) is provided without being isolated prior to forming a compound of Formula I.

In other embodiments, there is provided a method (xix), (xx), (xxi), (xxii), (xxiii), or (xxiv) wherein the above method (xii), (xiii), (xiv), (xv), (xvii), or (xviii) further comprises providing a compound of the Formula V:

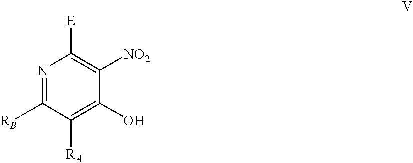

and reducing the compound of Formula V to provide a compound of Formula VI.

In other embodiments, there is provided a method (i-1), (ii-1), (iii-1), (iv-1), (v-1), (vi-1), (vii-1), (viii-1), (ix-1), (x-1), (xi-1), (xii-1), (xiii-1), (xiv-1), (xv-1), (xvi-1), (xvii-1), (xviii-1), (xix-1), (xx-1), (xxi-1), (xxii-1), (xxiii-1), or (xxiv-1) wherein the method (i), (ii), (iii), (iv), (v), (vi), (vii), (viii), (ix), (x), (xi), (xii), (xiii), (xiv), (xv), (xvi), (xvii), (xviii), (xix), (xx), (xxi), (xxii), (xxiii), or (xxiv), respectively, further comprises the step of converting E to an amino group in the compound of Formula I to provide a compound of the Formula X:

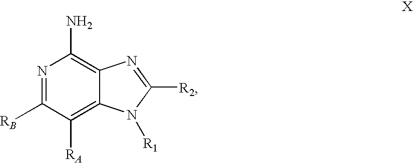

or a pharmaceutically acceptable salt thereof.

In other embodiments, there is provided a method (i-2), (ii-2), (iii-2), (iv-2), (v-2), (vi-2), (vii-2), (viii-2), (ix-2), (x-2), (xi-2), (xii-2), (xiii-2), (xiv-2), (xv-2), (xvi-2), (xvii-2), (xviii-2), (xix-2), (xx-2), (xxi-2), (xxii-2), (xxiii-2), or (xxiv-2) wherein E in the method (i-1), (ii-1), (iii-1), (iv-1), (v-1), (vi-1), (vii-1), (viii-1), (ix-1), (x-1), (xi-1), (xii-1), (xiii-1), (xiv-1), (xv-1), (xvi-1), (xvii-1), (xviii-1), (xix-1), (xx-1), (xxi-1), (xxii-1), (xxiii-1), or (xxiv-1) is hydrogen, the compound of Formula I is the Formula I-2:

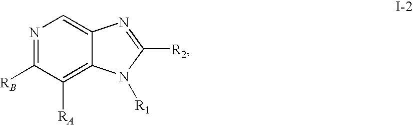

and the step of converting the hydrogen to an amino group in the compound of Formula I-2 comprises:

oxidizing the compound of Formula I-2 to provide a the 5N-oxide of Formula XX:

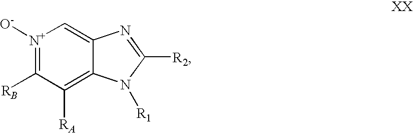

and aminating the compound of Formula XX to provide the compound of Formula X, or a pharmaceutically acceptable salt thereof.

In other embodiments, there is provided a method (i-3), (ii-3), (iii-3), (iv-3), (v-3), (vi-3), (vii-3), (viii-3), (ix-3), (x-3), (xi-3), (xii-3), (xiii-3), (xiv-3), (xv-3), (xvi-3), (xvii-3), (xviii-3), (xix-3), (xx-3), (xxi-3), (xxii-3), (xxiii-3), or (xxiv-3) wherein E in the method (i-1), (ii-1), (iii-1), (iv-1), (v-1), (vi-1), (vii-1), (viii-1), (ix-1), (x-1), (xi-1), (xii-1), (xiii-1), (xiv-1), (xv-1), (xvi-1), (xvii-1), (xviii-1), (xix-1), (xx-1), (xxi-1), (xxii-1), (xxiii-1), or (xxiv-1) is Hal, the compound of Formula I is the Formula I-3:

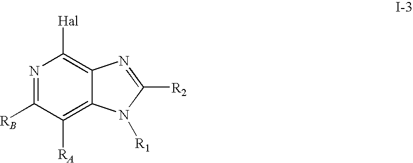

wherein Hal is fluoro, chloro, bromo, or iodo, and the step of converting the Hal group to an amino group in the compound of Formula I-3 comprises aminating the compound of Formula I-3 to provide the compound of Formula X, or a pharmaceutically acceptable salt thereof.

In other embodiments, there is provided a method (i-4), (ii-4), (iii-4), (iv-4), (v-4), (vi-4), (vii-4), (viii-4), (ix-4), (x-4), (xi-4), (xii-4), (xiii-4), (xiv-4), (xv-4), (xvi-4), (xvii-4), (xviii-4), (xix-4), (xx-4), (xxi-4), (xxii-4), (xxiii-4), or (xxiv-4) wherein E in the method (1-1), (ii-1), (iii-1), (iv-1), (v-1), (vi-1), (vii-1), (viii-1), (ix-1), (x-1), (xi-1), (xii-1), (xiii-1), (xiv-1), (xv-1), (xvi-1), (xvii-1), (xviii-1), (xix-1), (xx-1), (xxi-1), (xxii-1), (xxiii-1), or (xxiv-1) is hydroxy, the compound of Formula I is the Formula I-4:

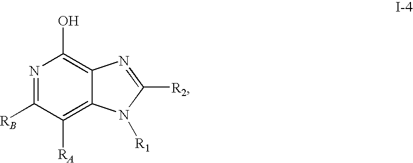

and the step of converting the hydroxy group to an amino group in the compound of Formula I-4 comprises:

converting the hydroxy group at the 4-position of Formula I-4 to a halo group to provide a compound or salt of Formula I-3:

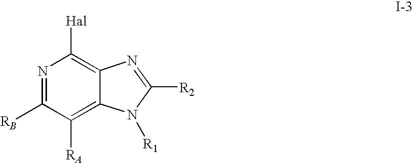

wherein Hal is fluoro, chloro, bromo, or iodo; and

aminating the compound of Formula I-3 to provide the compound of Formula X, or a pharmaceutically acceptable salt thereof.

In other embodiments, there is provided a method (i-5), (ii-5), (iii-5), (iv-5), (v-5), (vi-5), (vii-5), (viii-5), (ix-5), (x-5), (xi-5), (xii-5), (xiii-5), (xiv-5), (xv-5), (xvi-5), (xvii-5), (xviii-5), (xix-5), (xx-5), (xxi-5), (xxii-5), (xxiii-5), or (xxiv-5) wherein E in the method (i-1), (ii-1), (iii-1), (iv-1), (v-1), (vi-1), (vii-1), (viii-1), (ix-1), (x-1), (xi-1), (xii-1), (xiii-1), (xiv-1), (xv-1), (xvi-1), (xvii-1), (xviii-1), (xix-1), (xx-1), (xxi-1), (xxii-1), (xxiii-1), or (xxiv-1) is hydroxy, the compound of Formula I is the Formula I-4:

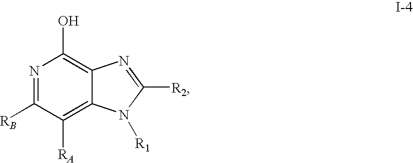

and the step of converting the hydroxy group to an amino group in the compound of Formula I-4 comprises:

sulfonating the compound of Formula I-4 by reaction with a compound of the formula hal-S(O)₂—R′ wherein hal is chloro or bromo, or the formula O(—S(O)₂—R′)₂, to provide a compound of the Formula I-5:

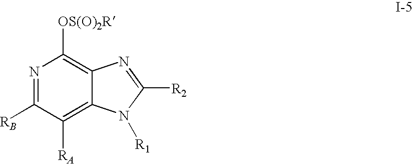

displacing the —O—S(O)₂—R′ group in Formula I-5 by an amino group of the formula —N(Bn)₂ to provide a compound of the Formula I-6:

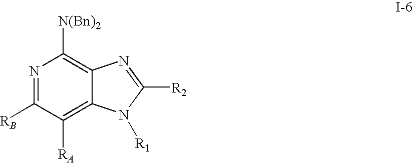

removing the Bn protecting groups in Formula I-6 to provide the compound of Formula X, or a pharmaceutically acceptable salt thereof.

In other embodiments, there is provided a method (i-6), (ii-6), (iii-6), (iv-6), (v-6), (vi-6), (vii-6), (viii-6), (ix-6), (x-6), (xi-6), (xii-6), (xiii-6), (xiv-6), (xv-6), (xvi-6), (xvii-6), (xviii-6), (xix-6), (xx-6), (xxi-6), (xxii-6), (xxiii-6), or (xxiv-6) wherein E in the method (i-1), (ii-1), (iii-1), (iv-1), (v-1), (vi-1), (vii-1), (viii-1), (ix-1), (x-1), (xi-1), (xii-1), (xiii-1), (xiv-1), (xv-1), (xvi-1), (xvii-1), (xviii-1), (xix-1), (xx-1), (xxi-1), (xxii-1), (xxiii-1), or (xxiv-1) is phenoxy, the compound of Formula I is the Formula I-7:

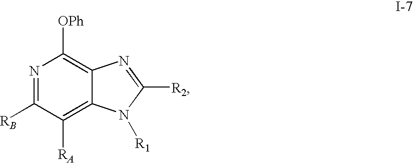

wherein Ph is phenyl, and the step of converting the phenoxy group to an amino group in the compound of Formula I-7 comprises aminating the compound of Formula I-7 to provide the compound of Formula X, or a pharmaceutically acceptable salt thereof.

In other embodiments, there is provided a method (i-7), (ii-7), (iii-7), (iv-7), (v-7), (vi-7), (vii-7), (viii-7), (ix-7), (x-7), (xi-7), (xii-7), (xiii-7), (xiv-7), (xv-7), (xvi-7), (xvii-7), (xviii-7), (xix-7), (xx-7), (xxi-7), (xxii-7), (xxiii-7), or (xxiv-7) wherein E in the method (i-1), (ii-1), (iii-1), (iv-1), (v-1), (vi-1), (vii-1), (viii-1), (ix-1), (x-1), (xi-1), (xii-1), (xiii-1), (xiv-1), (xv-1), (xvi-1), (xvii-1), (xviii-1), (xix-1), (xx-1), (xxi-1), (xxii-1), (xxiii-1), or (xxxiv-1) is —O—S(O)₂—R′, the compound of Formula I is the Formula I-5:

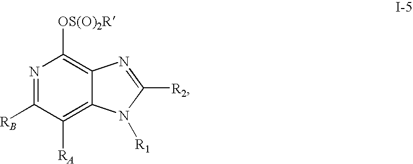

and the step of converting the —O—S(O)₂—R′ group to an amino group in the compound of Formula I-5 comprises:

displacing the —O—S(O)₂—R′ group by an amino group of the formula —N(Bn)₂ to provide a compound of the Formula I-6:

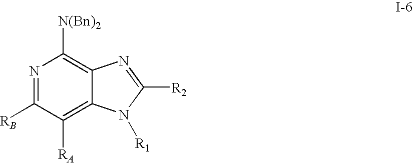

and removing the Bn protecting groups in Formula I-6 to provide the compound of Formula X, or a pharmaceutically acceptable salt thereof.

In other embodiments, there is provided a method (i-8), (ii-8), (iii-8), (iv-8), (v-8), (vi-8), (vii-8), (viii-8), (ix-8), (x-8), (xi-8), (xii-8), (xiii-8), (xiv-8), (xv-8), (xvi-8), (xvii-8), (xviii-8), (xix-8), (xx-8), (xxi-8), (xxii-8), (xxiii-8), or (xxiv-8) wherein E in the method (i-1), (ii-1), (iii-1), (iv-1), (v-1), (vi-1), (vii-1), (viii-1), (ix-1), (x-1), (xi-1), (xii-1), (xiii-1), (xiv-1), (xv-1), (xvi-1), (xvii-1), (xviii-1), (xix-1), (xx-1), (xxi-1), (xxii-1), (xxiii-1), or (xxiv-1) is —N(Bn)₂, the compound of Formula I is the Formula I-6:

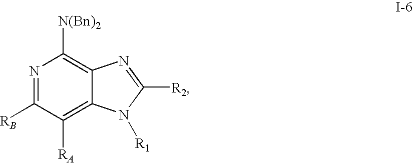

and the step of converting the —N(Bn)₂ group to an amino group in the compound of Formula I-6 comprises removing the Bn protecting groups to provide the compound of Formula X, or a pharmaceutically acceptable salt thereof.

In other embodiments, there is provided a method (i-9), (ii-9), (iii-9), (iv-9), (v-9), (vi-9), (vii-9), (viii-9), (ix-9), (x-9), (xi-9), (xii-9), (xiii-9), (xiv-9), (xv-9), (xvi-9), (xvii-9), (xviii-9), (xix-9), (xx-9), (xxi-9); (xxii-9), (xxiii-9), or (xxiv-9) wherein E in the method (i-1), (ii-1), (iii-1), (iv-1), (v-1), (vi-1), (vii-1), (viii-1), (ix-1), (x-1), (xi-1), (xii-1), (xiii-1), (xiv-1), (xv-1), (xvi-1), (xvii-1), (xviii-1), (xix-1), (xx-1), (xxi-1), (xxii-1), (xxiii-1), or (xxiv-1) is joined with the adjacent pyridine nitrogen atom of Formula I to form the fused tetrazolo ring in Formula I-1:

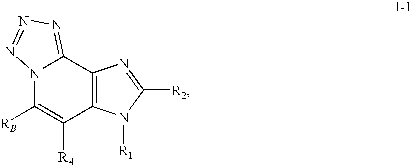

and the step of converting the fused tetrazolo ring to an amino group in the compound of Formula I-1 comprises the steps of:

reacting the compound of Formula I-1 with triphenylphosphine to provide a compound of the Formula XXI:

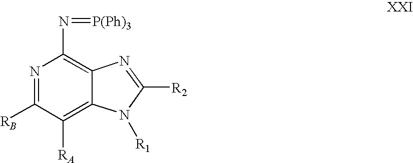

and hydrolyzing the compound of Formula XXI to provide the compound of Formula X, or a pharmaceutically acceptable salt thereof.

In other embodiments, there is provided a method (i-10), (ii-10), (iii-10), (iv-110), (v-10), (vi-10), (vii-10), (viii-10), (ix-10), (x-10), (xi-10), (xii-10), (xiii-10), (xiv-10), (xv-10), (xvi-10), (xvii-10), (xviii-10), (xix-10), (xx-10), (xxi-10), (xxii-10), (xxiii-10), or (xxiv-10) wherein E in the method (i-1), (ii-1), (iii-1), (iv-1), (v-1), (vi-1), (vii-1), (viii-1), (ix-1), (x-1), (xi-1), (xii-1), (xiii-1), (xiv-1), (xv-1), (xvi-1), (xvii-1), (xviii-1), (xix-1), (xx-1), (xxi-1), (xxii-1), (xxiii-1), or (xxiv-1) is joined with the adjacent pyridine nitrogen atom of Formula I to form the fused tetrazolo ring in Formula I-1:

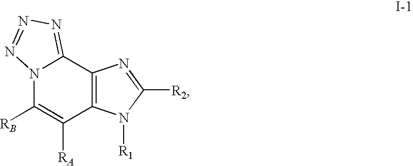

and the step of converting the fused tetrazolo ring to an amino group in the compound of Formula I-1 comprises the step of:

reductively removing the tetrazolo ring from the compound of Formula I-1 to provide the compound of Formula X, or a pharmaceutically acceptable salt thereof.

In another embodiment, the invention provides an intermediate compound of Formula XI:

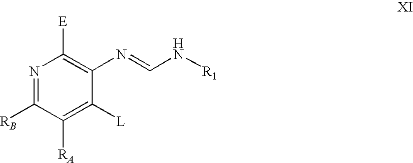

wherein:

E is selected from the group consisting of hydrogen, fluoro, chloro, bromo, iodo, hydroxy, phenoxy, —O—S(O)₂—R′, and —N(Bn)₂, wherein R′ is selected from the group consisting of alkyl, haloalkyl, and aryl optionally substituted by alkyl, halo, or nitro, and Bn is selected from the group consisting of benzyl, p-methoxybenzyl, p-methylbenzyl, and 2-furanylmethyl; or

E is joined with the adjacent pyridine nitrogen atom of Formula XI to form the fused tetrazolo ring in Formula XIII:

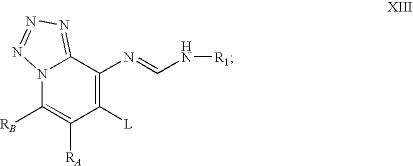

L is selected from the group consisting of fluoro, chloro, bromo, iodo, phenoxy, and —O—S(O)₂—R′, wherein R′ is selected from the group consisting of alkyl, haloalkyl, and aryl optionally substituted by alkyl, halo, or nitro;

R_{A} and R_{B} are independently selected from the group consisting of:
hydrogen,
halogen,
alkyl,
alkenyl,
alkoxy,
alkylthio, and
—N(R₉)₂;

or R_{A} and R_{B} taken together form a fused benzene ring or a fused pyridine ring wherein the benzene ring or pyridine ring is unsubstituted or substituted by one R group, or substituted by one R₃ group, or substituted by one R group and one R₃ group;

or R_{A} and R_{B} taken together form a fused 5 to 7 membered saturated ring optionally containing one nitrogen atom, wherein the fused ring is unsubstituted or substituted by one or more R groups;

R is selected from the group consisting of:
halogen,
hydroxy,
allyl,
alkenyl,
haloalkyl,
alkoxy,
alkylthio, and
—N(R₉)₂;

R₁ is selected from the group consisting of:
-R₄,
-X-R₄;
-X-Y-R₄,
-X-Y-X-Y-R₄,
-X-R₅,
—N(R₁′)-Q-R₄,
—N(R₁′)—X₁—Y₁—R₄, and
—N(R₁′)—X₁—R_{5b};

R₃ is selected from the group consisting of:
-Z-R₄,
-Z-X-R₄,
-Z-X-Y-R₄,
-Z-X-Y-X-Y-R₄, and
-Z-X-R₅;

X is selected from the group consisting of alkylene, alkenylene, alkynylene, arylene, heteroarylene, and heterocyclylene wherein the alkylene, alkenylene, and alkynylene groups can be optionally interrupted or terminated by arylene, heteroarylene or heterocyclylene and optionally interrupted by one or more —O— groups;

X₁ is C₂₋₂₀ alkylene;

Y is selected from the group consisting of:
—O—,
—S(O)₀₋₂—,
—S(O)₂—N(R₈)—,
—C(R₆)—,
—O—C(R₆)—,
—O—C(O)—O—,
—N(R₈)-Q-,
—O—C(R₆)—N(R₈)—,
—C(R₆)—N(OR₉)—,
—O—N(R₈)-Q-,
—O—N═C(R₄)—,
—C(═N—O—R₈)—,
—CH(—N(—O—R₈)-Q-R₄)—,

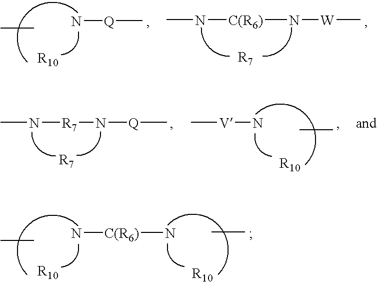

Y₁ is selected from the group consisting of —O—, —S(O)₀₋₂—, —S(O)₂—N(R₈)—, —N(R₈)-Q-, —C(R₆)—N(R₈)—, —O—C(R₆)—N(R₈)—, and

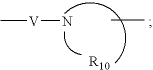

Z is a bond or —O—;

R₁′ is selected from the group consisting of hydrogen, C₁₋₂₀ alkyl, hydroxy-C₂₋₂₀ alkylenyl, and alkoxy-C₂₋₂₀ alkylenyl;

R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of allyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of allyl, alkenyl, alkynyl, and heterocyclyl, oxo;

R₅ is selected from the group consisting of:

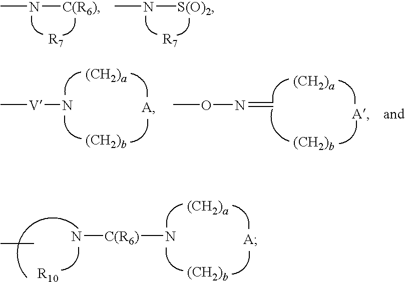

R_{5b} is selected from the group consisting of:

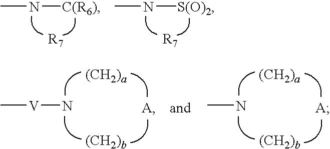

R₆ is selected from the group consisting of ═O and ═S;

R₇ is C₂₋₇ alkylene;

R₈ is selected from the group consisting of hydrogen, alkyl, alkoxyalkylenyl, hydroxyalkylenyl, arylalkylenyl, and heteroarylalkylenyl;

R₉ is selected from the group consisting of hydrogen and alkyl;

R₁₀ is C₃₋₈ alkylene;

A is selected from the group consisting of —O—, —C(O)—, —S(O)₀₋₂—, and —N(R₄)—;

A′ is selected from the group consisting of —O—, —S(O)₀₋₂—, —N(-Q-R₄)—, and —CH₂—;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, —C(R₆)—S—, and —C(R₆)—N(OR₉)—;

V is selected from the group consisting of —C(R₆)—, —O—C(R₆)—, —N(R₈)—C(R₆)—, and —S(O)₂—;

V′ is selected from the group consisting of —O—C(R₆)—, —N(R₈)—C(R₆)—, and —S(O)₂—;

W is selected from the group consisting of a bond, —C(O)—, and —S(O)₂—; and

a and b are independently integers from 1 to 6 with the proviso that a+b is ≦7;

or a pharmaceutically acceptable salt thereof.

For certain embodiments, including any one of the above embodiments, R₁ is selected from the group consisting of -R₄, -X-R₄, -X-Y-R₄, -X-Y-X-Y-R₄, -X-R₅, —N(R₁′)-Q-R₄, —N(R₁′)—X, -Y-R₄, and —N(R₁′)—X₁—R_{5b}.

For certain embodiments, including any one of the above embodiments, R₁ is selected from the group consisting of -R₄, -X-R₄, -X-Y-R₄, -X-Y-X-Y-R₄, and -X-R₅.

For certain embodiments, including any one of the above embodiments, R₁ is -R₄ or -X-R₄. For certain of these embodiments, -R₄ is selected from the group consisting of 2-methylpropyl, 2-hydroxy-2-methylpropyl, 2,2-dimethyl-4-oxopentyl, and (1-hydroxycyclobutyl)methyl. For certain of these embodiments, R₁ is -R₄, and -R₄ is 2-methylpropyl or 2-hydroxy-2-methylpropyl. For certain of these embodiments, R₁ is -R₄, and -R₄ is 2-methylpropyl. Alternatively, for certain of these embodiments, R₁ is -X-R₄, and -X-R₄ is 2,2-dimethyl-3-(2-methyl-1,3-dioxolan-2-yl)propyl.

For certain embodiments, including any one of the above embodiments except for embodiments where R₁ is -R₄ or -X-R₄, R₁ is -X-Y-R₄. For certain of these embodiments, X is C₂₋₄ allylene, and Y is —S(O)₂— or —N(R₈)-Q-. For certain of these embodiments, -X-Y-R₄ is selected from the group consisting of 2-(propylsulfonyl)ethyl, 2-methyl-2-[(methylsulfonyl)amino]propyl, 4-methylsulfonylaminobutyl, and 2-(acetylamino)-2-methylpropyl.

For certain embodiments, including any one of the above embodiments except for embodiments where R₁ is -R₄, -X-R₄, or -X-Y-R₄, R₁ is -X-R₅. For certain of these embodiments, -X-R₅ is 4-[(morpholin-4-ylcarbonyl)amino]butyl.

For certain embodiments, including any one of the above embodiments except for embodiments where R₁ is -R₄, -X-R₄, -X-Y-R₄, -X-Y-X-Y-R₄, or -X-R₅, R₁ is selected from the group consisting of —N(R₁′)-Q-R₄, —N(R₁′)—X₁—Y₁—R₄, and —N(R₁′)—X₁—R_{5b}.

For certain embodiments, including any one of the above embodiments except for embodiments where R₂ is hydrogen, R₂ is selected from the group consisting of -R₄, -X-R₄, -X-Y-R₄, and -X-R₅.

For certain embodiments, including any one of the above embodiments except for embodiments where excluded, R₂ is -R₄. For certain of these embodiments, R₂ is selected from the group consisting of hydrogen, methyl, ethyl, propyl, butyl, 2-methoxyethyl, 2-hydroxyethyl, ethoxymethyl, and hydroxymethyl. For certain of these embodiments, R₂ is selected from the group consisting of hydrogen, methyl, ethyl, and ethoxymethyl. For certain embodiments, R₂ is hydrogen.

For certain embodiments, including any one of the above embodiments, R_{A} and R_{B} are each independently selected from the group consisting of hydrogen, halogen, allyl, alkenyl, alkoxy, alkylthio, and —N(R₉)₂;

or R_{A} and R_{B} taken together form a fused benzene ring or a fused pyridine ring wherein the benzene ring or pyridine ring is unsubstituted or substituted by one R group, or substituted by one R₃ group, or substituted by one R group and one R₃ group;

or R_{A} and R_{B} taken together form a fused 5 to 7 membered saturated ring optionally containing one nitrogen atom, wherein the fused ring is unsubstituted or substituted by one or more R groups.

For certain embodiments, including any one of the above embodiments, R_{A} and R_{B} are each independently selected from the group consisting of hydrogen, halogen, alkyl, alkenyl, alkoxy, alkylthio, and —N(R₉)₂. For certain of these embodiments, R_{A} and R_{B} are each methyl.

For certain embodiments, including any one of the above embodiments where R_{A} and R_{B} taken together can form a fused benzene ring, R_{A} and R_{B} taken together form a fused benzene ring wherein the benzene ring is unsubstituted or substituted by one R group, or substituted by one R₃ group, or substituted by one R group and one R₃ group. For certain of these embodiments, R is hydroxy or bromo, and R₃ is methoxy, phenoxy, or benzyloxy. For certain of these embodiments, the fused benzene ring is substituted by one R group selected from the group consisting of hydroxy and bromo. Alternatively, for certain of these embodiments, the fused benzene ring is substituted by one R₃ group wherein R₃ is methoxy, phenoxy, or benzyloxy. For certain embodiments, R_{A} and R_{B} taken together form a fused benzene ring wherein the benzene ring is unsubstituted.

For certain embodiments, including any one of the above embodiments where R_{A} and R_{B} taken together can form a fused pyridine ring, R_{A} and R_{B} taken together form a fused pyridine ring wherein the fused pyridine ring is unsubstituted or substituted by one R group, or substituted by one R₃ group, or substituted by one R group and one R₃ group; and wherein the fused pyridine ring is

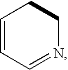

wherein the highlighted bond indicates the position where the ring is fused. For certain of these embodiments, R is hydroxy or bromo, and R₃ is methoxy, phenoxy, or benzyloxy. For certain of these embodiments, the fused pyridine ring is substituted by one R group selected from the group consisting of hydroxy and bromo. Alternatively, for certain of these embodiments, the fused pyridine ring is substituted by one R₃ group wherein R₃ is methoxy, phenoxy, or benzyloxy. For certain embodiments, R_{A} and R_{B} taken together form a fused pyridine ring wherein the fused pyridine ring is unsubstituted, and wherein the fused pyridine ring is

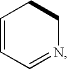

wherein the highlighted bond indicates the position where the ring is fused.

For certain embodiments, including any one of the above embodiments where R_{A} and R_{B} taken together can form a fused 5 to 7 membered saturated ring, R_{A} and R_{B} taken together form a fused 5 to 7 membered saturated ring optionally containing one nitrogen atom, wherein the fused ring is unsubstituted or substituted by one or more R groups. For certain of these embodiments, R_{A} and R_{B} taken together form a fused 5 to 7 membered carbocyclic ring wherein the fused ring is unsubstituted or substituted by one or more R groups. For certain of these embodiments, the fused ring is a 6 membered carbocyclic ring which is unsubstituted. Alternatively, for certain of these embodiments, R_{A} and R_{B} taken together form a fused 5 to 7 membered saturated ring containing one nitrogen atom, wherein the fused ring is unsubstituted or substituted by one or more R groups. For certain of these embodiments, the fused ring is a fused 6 membered ring which is unsubstituted or substituted at a carbon atom by one or more R groups. For certain of these embodiments, the fused 6 membered ring is

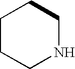

wherein the ring is unsubstituted; and wherein the highlighted bond indicates the position where the ring is fused.

For certain embodiments, including any one of the above embodiments of methods (i) through (viii), methods (i-1) through (viii-1), methods (i-2) through (viii-2), methods (i-3) through (viii-3), methods (i-4) through (viii-4), methods (i-5) through (viii-5), methods (i-6) through (viii-6), methods (i-7) through (viii-7), methods (i-8) through (viii-8), methods (i-9) through (viii-9), and methods (i-10) through (viii-10), R_{A} is R_{A1}, R_{B} is R_{B1}, R₁ is R₁ₐ, and R₂ is R₂ₐ, wherein:

R_{A1} and R_{B1} are independently selected from the group consisting of:
hydrogen,
halogen,
alkyl,
alkenyl,
alkoxy,
alkylthio, and
—N(R₉)₂;

or R_{A1} and R_{B1} taken together form a fused benzene ring or a fused pyridine ring wherein the benzene ring or pyridine ring is unsubstituted or substituted by one Rₐ group, or substituted by one R₃ₐ group, or substituted by one Rₐ group and one R₃ₐ group;

or R_{A1} and R_{B1} taken together form a fused 5 to 7 membered saturated ring optionally containing one nitrogen atom, wherein the fused ring is unsubstituted or substituted by one or more Rₐ groups;

Rₐ is selected from the group consisting of:
halogen,
hydroxy,
alkyl,
alkenyl,
trifluoromethyl,
alkoxy,
alkylthio, and
—N(R₉)₂;

R₁ₐ is selected from the group consisting of:
-R₄ₐ,
-X-R₄ₐ,
-X-Yₐ-R₄ₐ,
-X-Yₐ-X-Yₐ-R₄ₐ,
-X-R₅ₐ,
—N(R₁′)-Q-R₄ₐ,
—N(R₁′)—X₁—Y₁—R₄ₐ, and
—N(R₁′)—X₁—R_{5b};

R₂ₐ is selected from the group consisting of:
-R₄ₐ,
-X-R₄ₐ,
-X-Yₐ-R₄ₐ, and
-X-R₅ₐ;

R₃ₐ is selected from the group consisting of:
-Z-R₄ₐ,
-Z-X-R₄ₐ,
-Z-X-Yₐ-R₄ₐ,
-Z-X-Yₐ-X-Yₐ-R₄ₐ, and
-Z-X-R₅ₐ;

Yₐ is selected from the group consisting of:
—O—,
—S(O)₀₋₂—,
—S(O)₂—N(R₈)—,
—N(R₈)-Q-,
—O—C(R₆)—N(R₈)—,
—C(R₆)—N(OR₉)—,

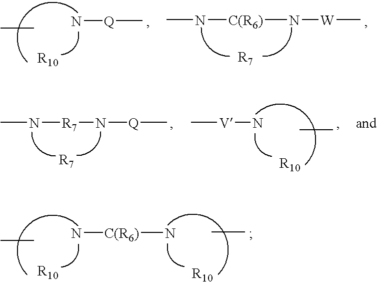

R₄ₐ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, trifluoromethyl, trifluoromethoxy, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, and (dialkylamino)alkyleneoxy; and

R₅₆ is selected from the group consisting of:

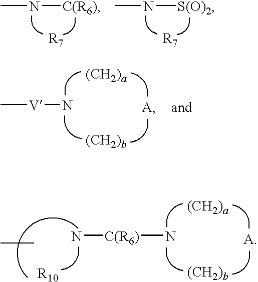

For each of the resulting embodiments, where present:

### Formula I is Formula Iₐ:

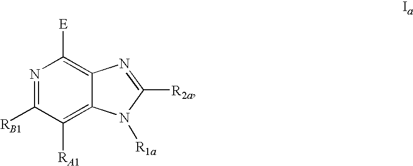

### Formula I-1 is Formula Iₐ-1:

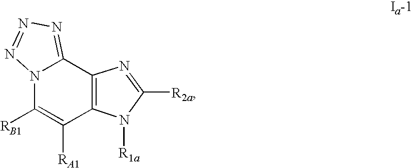

### Formula I-2 is Formula Iₐ-2:

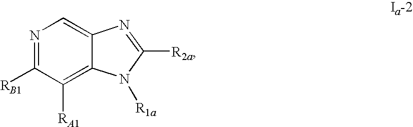

### Formula I-3 is Formula Iₐ-3:

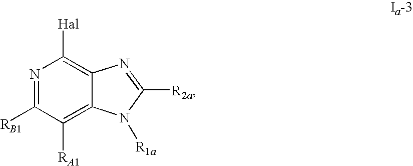

### Formula I-4 is Formula Iₐ-4:

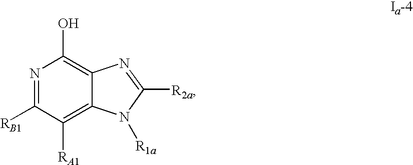

### Formula I-5 is Iₐ-5:

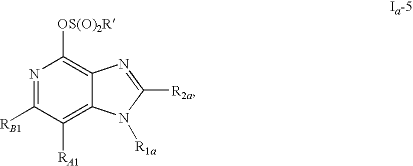

### Formula I-6 is Iₐ-6:

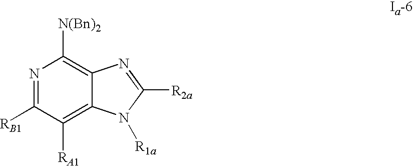

### Formula I-7 is Iₐ-7:

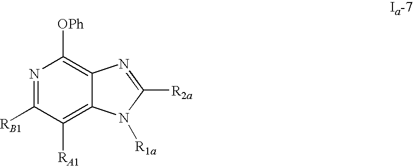

### Formula II is Formula IIₐ:

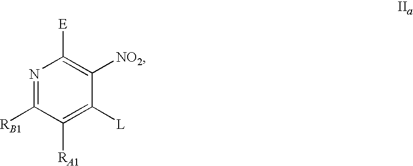

### Formula III is Formula IIIₐ:

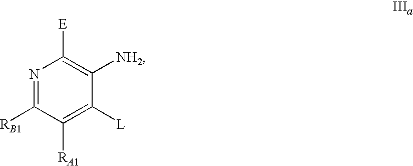

### Formula IV is Formula IVₐ:

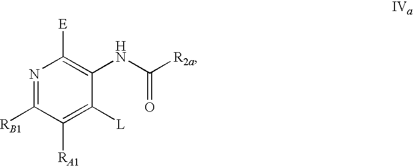

### Formula IV-1 is Formula IVₐ-1:

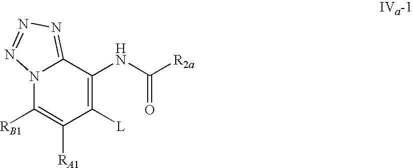

### Formula V is Formula Vₐ:

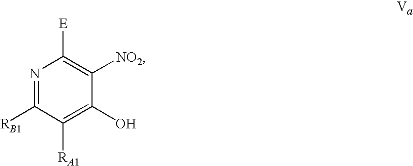

### Formula VI is Formula VIₐ:

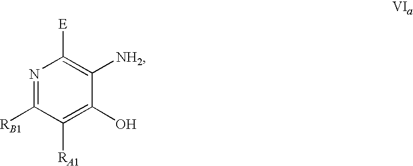

### and Formula VII is Formula VIIₐ:

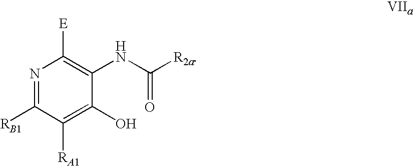

For certain of the resulting embodiments, where present:

### Formula X is Formula Xₐ:

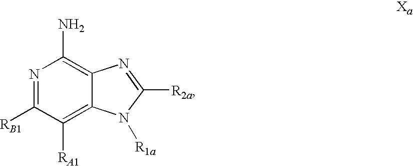

### Formula XX is Formula XXₐ:

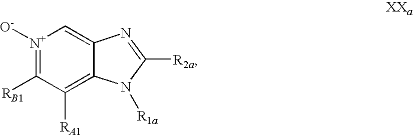

### and Formula XXI is Formula XXIₐ:

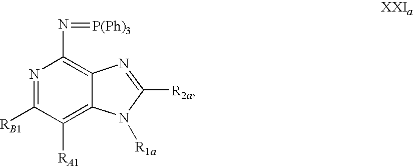

For certain embodiments, including any one of the above embodiments of methods (ix) through (xxiv), methods (ix-1) through (xxiv-1), methods (ix-2) through (xxiv-2), methods (ix-3) through (xxiv-3), methods (ix-4) through (xxiv-4), methods (ix-5) through (xxiv-5), methods (ix-6) through (xxiv-6), methods (ix-7) through (xxiv-7), methods (ix-8) through (xxiv-8), methods (ix-9) through (xxiv-9), and methods (ix-10) through (xxiv-10), R_{A} is R_{A1}, R_{B} is R_{B1}, R₁ is R₁ₐ, and R₂ is hydrogen, wherein R_{A1}, R_{B1}, and R₁ₐ are as defined above for embodiments wherein R_{A} is R_{A1}, R_{B} is R_{B1}, R₁ is R₁ₐ and R₂ is R₂ₐ. For the resulting embodiments of Formula Iₐ, Iₐ-2, Iₐ-3, Iₐ-4, Iₐ-5, Iₐ-6, and Iₐ-7, R₂ₐ is hydrogen. For resulting embodiments of Formula Xₐ, XXₐ, and XXIₐ, R₂ₐ is hydrogen.

For certain embodiments, including any one of the above embodiments of Formula XI, R_{A} is R_{A1}, R_{B} is R_{B1}, and R₁ is R₁ₐ, wherein R_{A1}, R_{B1}, and R₁ₐ are as defined above for embodiments wherein R_{A} is R_{A1}, R_{B} is R_{B1}, R₁ is R₁ₐ, and R₂ is R₂ₐ.

For each of the resulting embodiments, where present:

### Formula VIII is Formula VIIIₐ:

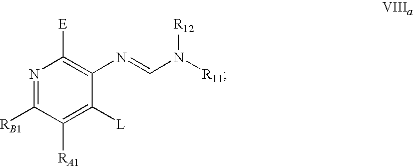

### Formula IX is Formula IXₐ:

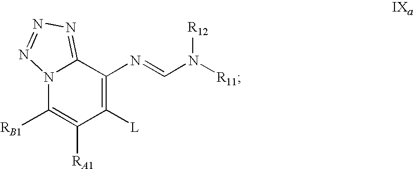

### Formula XI is Formula XIₐ:

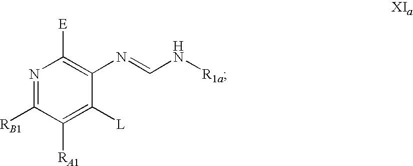

### and Formula XIII is Formula XIIIₐ:

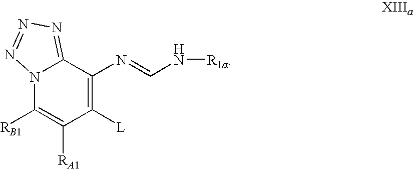

For each of the resulting embodiments, where present: R is Rₐ, R₃ is R₃ₐ, R is R₄ₐ R₅ is R₅ₐ, and Y is Yₐ.

For certain embodiments, including any one of the above embodiments where R_{A} is R_{A1}, R_{B} is R_{B1}, R₁ is R₁ₐ, and R₂ is R₂ₐ, embodiments where R_{A} is R_{A1}, R_{B} is R_{B1}, R₁ is R₁ₐ, and R₂ is hydrogen, and embodiments of XIₐ, R₁ₐ is selected from the group consisting of -R₄ₐ, -X-R₄ₐ, -X-Yₐ-R₄ₐ, -X-Yₐ-X-Yₐ-R₄ₐ, -X-R₅ₐ, —N(R₁′)-Q-R₄ₐ, —N(R₁′)-X₁—Y₁-R₄ₐ, and —N(R₁′)-X₁—R_{5b}.

For certain embodiments, including any one of the above embodiments where R_{A} is R_{A1}, R_{B} is R_{B1}, R₁ is R₁ₐ, and R₂ is R₂ₐ, embodiments where R_{A} is R_{A1}, R_{B} is R_{B1}, R₁ is R₁ₐ, and R₂ is hydrogen, and embodiments of XIₐ, R₁ₐ is selected from the group consisting of -R₄ₐ, -X-R₄ₐ, -X-Yₐ-R₄ₐ, -X-Yₐ-X-Yₐ-R₄ₐ, and -X-R₅ₐ.

For certain embodiments, including any one of the above embodiments where R_{A} is R_{A1}, R_{B} is R_{B1}, R₁ is R₁ₐ, and R₂ is R₂ₐ, embodiments where R_{A} is R_{A1}, R_{B} is R_{B1}, R₁ is R₁ₐ, and R₂ is hydrogen, and embodiments of XIₐ, R₁ₐ is -R₄ₐ or -X-R₄ₐ. For certain of these embodiments, -R₄ₐ is selected from the group consisting of 2-methylpropyl, 2-hydroxy-2-methylpropyl, and (1-hydroxycyclobutyl)methyl, and -X-R₄ₐ is 2,2-dimethyl-3-(2-methyl-1,3-dioxolan-2-yl)propyl. For certain of these embodiments, R₁ₐ is -R₄ₐ, and -R₄ₐ is 2-methylpropyl or 2-hydroxy-2-methylpropyl. For certain of these embodiments, R₁ₐ is -R₄ₐ, and -R₄ₐ is 2-methylpropyl. Alternatively, for certain of these embodiments, R₁ₐ is -X-R₄ₐ, and -X-R₄ₐ is 2,2-dimethyl-3-(2-methyl-1,3-dioxolan-2-yl)propyl.

For certain embodiments, including any one of the above embodiments where R_{A} is R_{A1}, R_{B} is R_{B1}, R₁ is R₁ₐ, and R₂ is R₂ₐ, embodiments where R_{A} is R_{A1}, R_{B} is R_{B1}, R₁ is R₁ₐ, and R₂ is hydrogen, and embodiments of XIₐ, except for embodiments where R₁ₐ is -R₄ₐ or -X-R₄ₐ, R₁ₐ is -X-Yₐ-R₄ₐ. For certain of these embodiments, X is C₂₋₄ alkylene, and Yₐ is —S(O)₂— or —N(R₈)-Q-. For certain of these embodiments, -X-Yₐ-R₄ₐ is selected from the group consisting of 2-(propylsulfonyl)ethyl, 2-methyl-2[(methylsulfonyl)amino]propyl, 4-methylsulfonylaminobutyl, and 2-(acetylamino)-2-methylpropyl.

For certain embodiments, including any one of the above embodiments where R_{A} is R_{A1}, R_{B} is R_{B1}, R₁ is R₁ₐ, and R₂ is R₂ₐ, embodiments where R_{A} is R_{A1}, R_{B} is R_{B1}, R₁ is R₁ₐ, and R₂ is hydrogen, and embodiments of XIₐ, except for embodiments where R₁ₐ is -R₄ₐ, -X-R₄ₐ, or -X-Yₐ-R₄ₐ, R₁ₐ is -X-R₅ₐ. For certain of these embodiments, -X-R₅ₐ is 4-[(morpholin-4-ylcarbonyl)amino]butyl.

For certain embodiments, including any one of the above embodiments where R_{A} is R_{A1}, R_{B} is R_{B1}, R₁ is R₁ₐ, and R₂ is R₂ₐ, embodiments where R_{A} is R_{A1}, R_{B} is R_{B1}, R₁ is R₁ₐ, and R₂ is hydrogen, and embodiments of XIₐ, except for embodiments where R₁ₐ is -R₄ₐ, -X-R₄ₐ, -X-Yₐ-R₄ₐ, -X-Yₐ-X-Yₐ-R₄ₐ, or -X-R₅ₐ, R₁ₐ is selected from the group consisting of —N(R₁′)-Q-R₄ₐ, —N(R₁′)—X₁—Y₁—R₄ₐ, and —N(R₁′)—X₁—R_{1b}.

For certain embodiments, including any one of the above embodiments where R_{A} is R_{A1}, R_{B} is R_{B1}, R₁ is R₁ₐ, and R₂ is R₂ₐ, R₂ₐ is selected from the group consisting of -R₄ₐ, -X-R₄ₐ, -X-Yₐ-R₄ₐ, and -X-R₅ₐ.

For certain embodiments, including any one of the above embodiments where R_{A} is R_{A1}, R_{B} is R_{B1}, R₁ is R₁ₐ, and R₂ is R₂ₐ, R₂ₐ is -R₄ₐ. For certain of these embodiments, R₂ₐ is selected from the group consisting of hydrogen, methyl, ethyl, propyl, butyl, 2-methoxyethyl, 2-hydroxyethyl, ethoxymethyl, and hydroxymethyl. For certain of these embodiments, R₂ₐ is selected from the group consisting of hydrogen, methyl, ethyl, and ethoxymethyl.

For certain embodiments, including any one of the above embodiments where R_{A} is R_{A1}, R_{B} is R_{B1}, R₁ is R₁ₐ, and R₂ is R₂ₐ, embodiments where R_{A} is R_{A1}, R_{B} is R_{B1}, R₁ is R₁ₐ, and R₂ is hydrogen, and embodiments of XIₐ, R_{A1} and R_{B1} are each independently selected from the group consisting of hydrogen, halogen, alkyl, alkenyl, alkoxy, alkylthio, and —N(R₉)₂;

or R_{A1} and R_{B1} taken together form a fused benzene ring or a fused pyridine ring wherein the benzene ring or pyridine ring is unsubstituted or substituted by one Rₐ group, or substituted by one R₃ₐ group, or substituted by one Rₐ group and one R₃ₐ group;

or R_{A1} and R_{B1} taken together form a fused 5 to 7 membered saturated ring optionally containing one nitrogen atom, wherein the fused ring is unsubstituted or substituted by one or more Rₐ groups.

For certain embodiments, including any one of the above embodiments where R_{A} is R_{A1}, R_{B} is R_{B1}, R₁ is R₁ₐ, and R₂ is R₂ₐ, embodiments where R_{A} is R_{A1}, R_{B} is R_{B1}, R₁ is R₁ₐ, and R₂ is hydrogen, and embodiments of XIₐ, R_{A1} and R_{B1} are each independently selected from the group consisting of hydrogen, halogen, allyl, alkenyl, alkoxy, alkylthio, and —N(R₉)₂. For certain of these embodiments, R_{A1} and R_{B1} are each methyl.

For certain embodiments, including any one of the above embodiments where R_{A} is R_{A1}, R_{B} is R_{B1}, R₁ is R₁ₐ, and R₂ is R₂ₐ, embodiments where R_{A} is R_{A1}, R_{B} is R_{B1}, R₁ is R₁ₐ, and R₂ is hydrogen, and embodiments of XIₐ, and where R_{A1} and R_{B1} taken together can form a fused benzene ring, R_{A1} and R_{B1} taken together form a fused benzene ring wherein the benzene ring is unsubstituted or substituted by one Rₐ group, or substituted by one R₃ₐ group, or substituted by one Rₐ group and one R₃ₐ group. For certain of these embodiments, Rₐ is hydroxy or bromo, and R₃ₐ is methoxy, phenoxy, or benzyloxy. For certain of these embodiments, the fused benzene ring is substituted by one Rₐ group selected from the group consisting of hydroxy and bromo. Alternatively, for certain of these embodiments, the fused benzene ring is substituted by one R₃ₐ group wherein R₃ₐ is methoxy, phenoxy, or benzyloxy. For certain embodiments, R_{A1} and R_{B1} taken together form a fused benzene ring that is unsubstituted.

For certain embodiments, including any one of the above embodiments where R_{A} is R_{A1}, R_{B} is R_{B1}, R₁ is R₁ₐ, and R₂ is R₂ₐ, embodiments where R_{A} is R_{A1}, R_{B} is R_{B1}, R₁ is R₁ₐ, and R₂ is hydrogen, and embodiments of XIₐ, and where R_{A1} and R_{B1} taken together can form a fused pyridine ring, R_{A1} and R_{B1} taken together form a fused pyridine ring wherein the fused pyridine ring is unsubstituted or substituted by one Rₐ group, or substituted by one R₃ₐ group, or substituted by one Rₐ group and one R₃ₐ group; and wherein the fused pyridine ring is

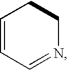

wherein the highlighted bond indicates the position where the ring is fused. For certain of these embodiments, Rₐ is hydroxy or bromo, and R₃ₐ is methoxy, phenoxy, or benzyloxy. For certain of these embodiments, the fused pyridine ring is substituted by one Rₐ group selected from the group consisting of hydroxy and bromo. Alternatively, for certain of these embodiments, the fused pyridine ring is substituted by one R₃ₐ group wherein R₃ₐ is methoxy, phenoxy, or benzyloxy. Alternatively, for certain of these embodiments, the fused pyridine ring is unsubstituted.

For certain embodiments, including any one of the above embodiments where R_{A} is R_{A1}, R_{B} is R_{B1}, R₁ is R₁ₐ, and R₂ is R₂ₐ, embodiments where R_{A} is R_{A1}, R_{B} is R_{B1}, R₁ is R₁ₐ, and R₂ is hydrogen, and embodiments of XIₐ, and where R_{A1} and R_{B1} taken together can form a fused 5 to 7 membered saturated ring, R_{A1} and R_{B1} taken together form a fused 5 to 7 membered saturated ring optionally containing one nitrogen atom, wherein the fused ring is unsubstituted or substituted by one or more Rₐ groups. For certain of these embodiments, R_{A1} and R_{B1} taken together form a fused 5 to 7 membered carbocyclic ring wherein the fused ring is unsubstituted or substituted by one or more Rₐ groups. For certain of these embodiments, the fused ring is a 6 membered carbocyclic ring which is unsubstituted. Alternatively, for certain of these embodiments, R_{A1} and R_{B1} taken together form a fused 5 to 7 membered saturated ring containing one nitrogen atom, wherein the fused ring is unsubstituted or substituted by one or more Rₐ groups. For certain of these embodiments, the fused ring is a fused 6 membered ring which is unsubstituted or substituted at a carbon atom by one or more Rₐ groups. For certain of these embodiments, the fused 6 membered ring is

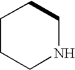

wherein the ring is unsubstituted; and wherein the highlighted bond indicates the position where the ring is fused.

For certain embodiments, including any one of the above embodiments which includes a step of reacting the compound of Formula IV with an amine of the formula R₁NH₂, the step is carried out neat and at an elevated temperature.

For certain embodiments, including any one of the above embodiments wherein Formula IV is the Formula IVₐ:

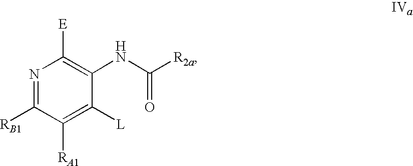

and which includes a step of reacting the compound of Formula IVₐ with an amine of the formula R₁ₐNH₂, the step is carried out neat and at an elevated temperature.

For certain embodiments, including any one of the above embodiments which includes a step of reacting the compound of Formula IV with an amine of the formula R₁NH₂, the step is carried out in a solvent and at an elevated temperature, except for embodiments wherein the step is carried out neat. For certain of these embodiments, the solvent is selected from the group consisting of methanol, ethanol, trifluoroethanol, isopropanol, tert-butanol, water, acetonitrile, 1-methyl-2-pyrrolidinone, and toluene. For certain of these embodiments, the solvent is selected from the group consisting of trifluoroethanol, isopropanol, and tert-butanol.

For certain embodiments, including any one of the above embodiments wherein Formula IV is the Formula IVₐ:

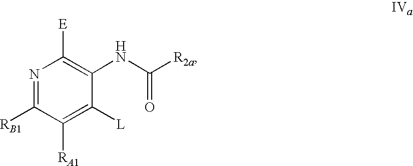

and which includes a step of reacting the compound of Formula IVₐ with an amine of the formula R₁ₐNH₂, the step is carried out in a solvent and at an elevated temperature, except for embodiments wherein the step is carried out neat. For certain of these embodiments, the solvent is selected from the group consisting of methanol, ethanol, trifluoroethanol, isopropanol, tert-butanol, water, acetonitrile, 1-methyl-2-pyrrolidinone, and toluene. For certain of these embodiments, the solvent is selected from the group consisting of trifluoroethanol, isopropanol, and tert-butanol.

For certain embodiments, including any one of the above embodiments which includes a step of reacting the compound of Formula VIII with an amine of the formula R₁NH₂, the step is carried out neat. For certain of these embodiments, the amine is of the formula R₁ₐNH₂. For certain of these embodiments, the step is carried out at an elevated temperature. For certain of these embodiments, the compound of Formula VIII is of Formula VIIIₐ.

For certain embodiments, including any one of the above embodiments which includes a step of reacting the compound of Formula VIII with an amine of the formula R₁NH₂, the step is carried out in a solvent. For certain of these embodiments, the solvent is selected from the group consisting of methanol, ethanol, trifluoroethanol, isopropanol, tert-butanol, water, acetonitrile, 1-methyl-2-pyrrolidinone, toluene, and tetrahydrofuran. For certain of these embodiments, the solvent is selected from the group consisting of trifluoroethanol, isopropanol, tert-butanol, and acetonitrile. For certain of these embodiments, the amine is of the formula R₁ₐNH₂. For certain of these embodiments, the step is carried out at an elevated temperature. For certain other of these embodiments, the step is carried out at room temperature. For certain of these embodiments, the compound of Formula VIII is of Formula VIIIₐ.

For certain embodiments, including any one of the above embodiments which includes an elevated temperature, the elevated temperature is not lower than 80° C.

For certain embodiments, including any one of the above embodiments which includes an elevated temperature, the elevated temperature is not lower than 110° C.

For certain embodiments, including any one of the above embodiments which includes an elevated temperature, the elevated temperature is not higher than 200° C.

For certain embodiments, including any one of the above embodiments which includes an elevated temperature, the elevated temperature is not higher than 180° C.

For certain embodiments, including any one of the above embodiments which includes an elevated temperature, the elevated temperature is not higher than 165° C.

For certain embodiments, including any one of the above embodiments which includes an elevated temperature, the elevated temperature is not higher than 150° C.

For certain embodiments, including anyone of the above embodiments which includes an elevated temperature, the elevated temperature is not higher than 135° C.

In one embodiment, the present invention provides a compound of the Formula IV:

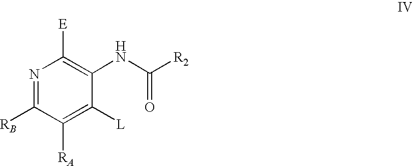

wherein R_{A}, R_{B}, R₂, E, and L are as defined above in method (i). For certain of these embodiments, R_{A}, R_{B}, R₂, E, and L are as defined in any one of the above embodiments of method (i).

In another embodiment, the present invention provides a compound of the Formula IVₐ:

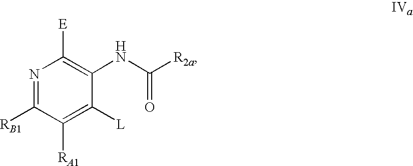

wherein R_{A1}, R_{B1}, R₂ₐ, E, and L are as defined above in method (i) where R_{A} is R_{A1}, R_{B} is R_{B1}, and R₂ is R₂ₐ. For certain of these embodiments, R_{A1}, R_{B1}, R₂ₐ, E, and L are as defined in any one of the above embodiments of method (i) where R_{A} is R_{A1}, R_{B} is R_{B1}, and R₂ is R₂ₐ.

For certain embodiments, including any one of the above embodiments of method (i), (ii), (iii), (iv), (v), (vi), (vii), (viii), or Formula IV or IVₐ, E is selected from the group consisting of hydrogen, fluoro, chloro, bromo, iodo, hydroxy, phenoxy, —O—S(O)₂—R′, and —N(Bn)₂, wherein R′ is selected from the group consisting of alkyl, haloalkyl, and aryl optionally substituted by alkyl, halo, or nitro, and Bn is selected from the group consisting of benzyl, p-methoxybenzyl, p-methylbenzyl, and 2-furanylmethyl; or E is joined with the adjacent pyridine nitrogen atom to form the fused tetrazolo ring shown in Formulas I-1 and IV-1:

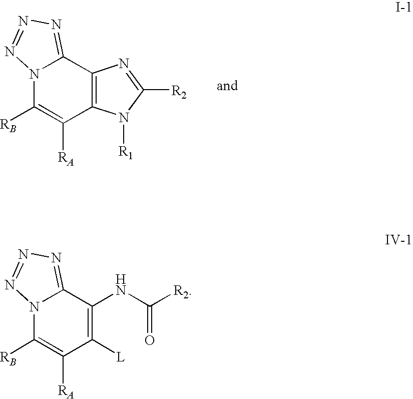

For certain embodiments, including any one of the above embodiments of method (ix), (x), (xi), (xii), (xiii), (xiv), (xv), (xix), (xx), (xxi), or (xxii), E is selected from the group consisting of hydrogen, fluoro, chloro, bromo, iodo, hydroxy, phenoxy, —O—S(O)₂—R′, and —N(Bn)₂, wherein R′ is selected from the group consisting of alkyl, haloalkyl, and aryl optionally substituted by alkyl, halo, or nitro, and Bn is selected from the group consisting of benzyl, p-methoxybenzyl, p-methylbenzyl, and 2-furanylmethyl; or

E is joined with the adjacent pyridine nitrogen atom of Formulas I and VIII to form the fused tetrazolo ring in Formulas I-1 and IX:

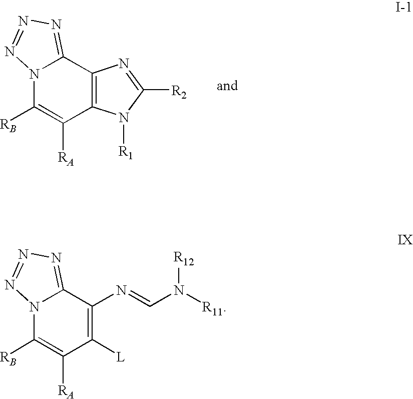

For certain embodiments, including any one of the above embodiments of method (xvi), (xvii), (xviii), (xxiii), and (xxiv) E is selected from the group consisting of hydrogen, fluoro, chloro, bromo, iodo, hydroxy, phenoxy, —O—S(O)₂—R′, and —N(Bn)₂, wherein R′ is selected from the group consisting of alkyl, haloalkyl, and aryl optionally substituted by alkyl, halo, or nitro, and Bn is selected from the group consisting of benzyl, p-methoxybenzyl, p-methylbenzyl, and 2-furanylmethyl; or

E is joined with the adjacent pyridine nitrogen atom of Formulas I and XI to form the fused tetrazolo ring in Formulas I-1 and XIII:

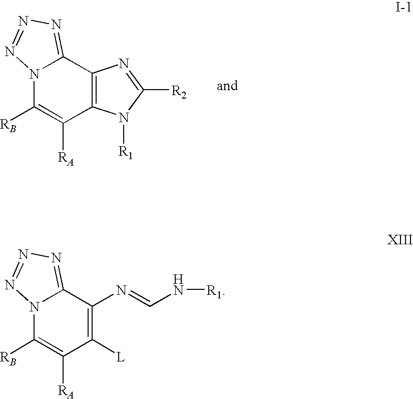

For certain embodiments, including any one of the above embodiments of Formula XI or XIₐ, E is selected from the group consisting of hydrogen, fluoro, chloro, bromo, iodo, hydroxy, phenoxy, —O—S(O)₂—R′, and —N(Bn)₂, wherein R′ is selected from the group consisting of alkyl, haloalkyl, and aryl optionally substituted by alkyl, halo, or nitro, and Bn is selected from the group consisting of benzyl, p-methoxybenzyl, p-methylbenzyl, and 2-furanylmethyl; or

E is joined with the adjacent pyridine nitrogen atom of Formula XI to form the fused tetrazolo ring in Formula XIII:

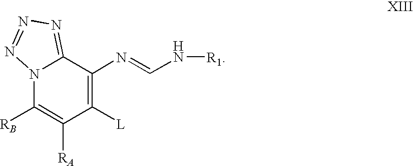

For certain of these embodiments, E is hydrogen. Alternatively, for certain of these embodiments, E is fluoro, chloro, bromo, or iodo, and for certain of these embodiments, E is chloro. Alternatively, for certain of these embodiments, E is hydroxy. Alternatively, for certain of these embodiments, E is phenoxy (OPh). Alternatively, for certain of these embodiments, E is —O—S(O)₂—R′ wherein R′ is selected from the group consisting of alkyl, haloalkyl, and aryl optionally substituted by alkyl, halo, or nitro. Alternatively, for certain of these embodiments, E is —N(Bn)₂ wherein Bn is selected from the group consisting of benzyl, p-methoxybenzyl, p-methylbenzyl, and 2-furanylmethyl. Alternatively, for certain of these embodiments, E is joined with the adjacent pyridine nitrogen atom to form the fused tetrazolo ring.

For certain embodiments, including any one of the above embodiments, L is selected from the group consisting of fluoro, chloro, bromo, iodo, phenoxy, and —O—S(O)₂—R′, wherein R′ is selected from the group consisting of alkyl, haloalkyl, and aryl optionally substituted by allyl, halo, or nitro. For certain embodiments, including any one of the above embodiments, L is selected from the group consisting of fluoro, chloro, bromo, iodo, phenoxy, and —O—S(O)₂—R′, wherein R′ is selected from the group consisting of alkyl, haloalkyl, and aryl optionally substituted by alkyl or halo. For certain of these embodiments, L is fluoro, chloro, bromo, or iodo, and for certain of these embodiments, L is chloro. Alternatively, for certain of these embodiments, L is phenoxy. Alternatively, for certain of these embodiments, L is —O—S(O)₂—R′, wherein R′ is selected from the group consisting of alkyl, haloalkyl, and aryl optionally substituted by alkyl, halo, or nitro. For certain of these embodiments, L is —O—S(O)₂—R′, wherein R′ is selected from the group consisting of alkyl, haloalkyl, and aryl optionally substituted by alkyl or halo.

For certain embodiments, including any one of the above embodiments wherein R′ is present, R′ is selected from the group consisting of alkyl, haloalkyl, and aryl optionally substituted by alkyl, halo, or nitro. For certain embodiments, R′ is selected from the group consisting of allyl, haloalkyl, and aryl optionally substituted by alkyl or halo. For certain of these embodiments, R′ is alkyl, and for certain of these embodiments, R′ is methyl. Alternatively, for certain of these embodiments, R′ is haloalkyl, and for certain of these embodiments, R′ is trifluoromethyl. Alternatively, for certain of these embodiments, R′ is aryl optionally substituted by alkyl or halo, and for certain of these embodiments, R′ is phenyl, p-bromophenyl, or p-tolyl. For certain of these embodiments, R′ is aryl optionally substituted by alkyl, halo, or nitro, and for certain of these embodiments, R′ is phenyl, p-bromophenyl, p-tolyl, 2-nitrophenyl, or 4-nitrophenyl.

For certain embodiments, including any one of the above embodiments wherein R is present, R is selected from the group consisting of halogen, hydroxy, alkyl, alkenyl, haloalkyl, alkoxy, alkylthio, and —N(R₉)₂. For certain of these embodiments, R is selected from the group consisting of hydroxy and bromo. For certain of these embodiments, R is at the 7- or 8-position. For certain of these embodiments, R is at the 7-position. Alternatively, for certain of these embodiments, R is at the 8-position.

For certain embodiments, including any one of the above embodiments wherein R is present except where R is selected from the group consisting of hydroxy and bromo, R is Rₐ.

For certain embodiments, including any one of the above embodiments wherein R₁ is present, Rₐ is selected from the group consisting of halogen, hydroxy, alkyl, alkenyl, trifluoromethyl, alkoxy, alkylthio, and —N(R₉)₂. For certain of these embodiments, Rₐ is selected from the group consisting of hydroxy and bromo. For certain of these embodiments, Rₐ is at the 7- or 8-position. For certain of these embodiments, Rₐ is at the 7-position. Alternatively, for certain of these embodiments, Rₐ is at the 8-position.

For certain embodiments, including any one of the above embodiments wherein R₃ is present, R₃ is selected from the group consisting of -Z-R₄, -Z-X-R₄, -Z-X-Y-R₄, -Z-X-Y-X-Y-R₄, and -Z-X-R₅. For certain of these embodiments, R₃ is -Z-R₄ or -Z-X-R₄.

For certain of these embodiments, R₃ is -Z-R₄. Alternatively, for certain of these embodiments, R₃ is -Z-X-R₄. For certain of any of these embodiments, Z is —O—. For certain of these embodiments, R₃ is methoxy, phenoxy, or benzyloxy. For certain of these embodiments, R₃ is at the 7- or 8-position. For certain of these embodiments, R₃ is at the 7-position. For certain of these embodiments, R₃ is a benzyloxy group at the 7-position. Alternatively, for certain of these embodiments, R₃ is at the 8-position.

For certain embodiments, including any one of the above embodiments wherein R₃ is present except where R₃ is -Z-R₄, -Z-X-R₄, methoxy, phenoxy, or benzyloxy, R₃ is R₃ₐ.

For certain embodiments, including any one of the above embodiments wherein R₃ₐ is present, R₃ₐ is selected from the group consisting of -Z-R₄ₐ, -Z-X-R₄ₐ, -Z-X-Yₐ-R₄ₐ, -Z-X-Yₐ-X-Yₐ-R₄ₐ, and -Z-X-R₅ₐ. For certain of these embodiments, R₃ₐ is -Z-R₄ₐ or -Z-X-R₄ₐ. For certain of these embodiments, R₃ₐ is -Z-R₄ₐ, Alternatively, for certain of these embodiments, R₃ₐ is -Z-X-R₄ₐ. For certain of any of these embodiments, Z is —O—. For certain of these embodiments, R₃ₐ is methoxy, phenoxy, or benzyloxy. For certain of these embodiments, R₃ₐ is at the 7- or 8-position. For certain of these embodiments, R₃ₐ is at the 7-position. For certain of these embodiments, R₃ₐ is a benzyloxy group at the 7-position. Alternatively, for certain of these embodiments, R₃ₐ is at the 8-position.

For certain embodiments, including any one of the above embodiments wherein R₄ is present, R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo. For certain of these embodiments, R₄ is alkyl optionally substituted by hydroxy or oxo. For certain of these embodiments, R₄ is selected from the group consisting of 2-methylpropyl, 2-hydroxy-2-methylpropyl, 2,2-dimethyl-4-oxopentyl, and (1-hydroxycyclobutyl)methyl. For certain of these embodiments, R₄ is 2-methylpropyl or 2-hydroxy-2-methylpropyl. For certain of these embodiments, R₄ is 2-methylpropyl.

For certain embodiments, including any one of the above embodiments wherein R₄ is present in -X-Y-R₄, R₄ is C₁₋₄ alkyl. For certain of these embodiments, R₄ is methyl.

For certain embodiments, including any one of the above embodiments wherein R₄ₐ is present, R₄ₐ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, trifluoromethyl, trifluoromethoxy, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, and (dialkylamino)alkyleneoxy. For certain of these embodiments, R₄ₐ is allyl optionally substituted by hydroxy. For certain of these embodiments, R₄ₐ is selected from the group consisting of 2-methylpropyl, 2-hydroxy-2-methylpropyl, and (1-hydroxycyclobutyl)methyl. For certain of these embodiments, R₄ₐ is 2-methylpropyl or 2-hydroxy-2-methylpropyl. For certain of these embodiments, R₄ₐ is 2-methylpropyl.

For certain embodiments, including any one of the above embodiments wherein R₄ₐ is present in -X-Y-R₄ₐ, R₄ₐ is C₁₋₄ alkyl. For certain of these embodiments, R₄ₐ is methyl.

For certain embodiments, including any one of the above embodiments wherein R₅ is present, R₅ is selected from the group consisting of:

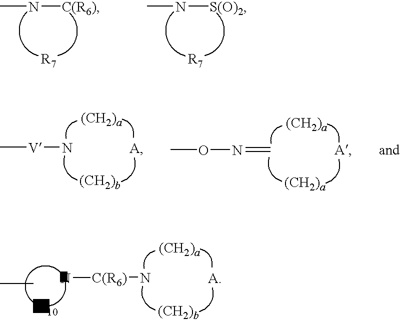

For certain of these embodiments, R₅ is

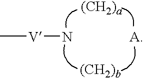

For certain of these embodiments, V′ is —NH—C(O)—. For certain of these embodiments, A is —O—. For certain of these embodiments, a and b are each 2.

For certain embodiments, including any one of the above embodiments wherein R₅ is present, R₅ is R₅ₐ.

For certain embodiments, including any one of the above embodiments wherein R₅ₐ is present, R₅ₐ is selected from the group consisting of:

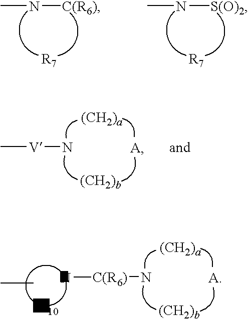

For certain of these embodiments, R₅ₐ is

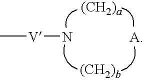

For certain of these embodiments, V′ is —NH—C(O)—. For certain of these embodiments, A is —O—. For certain of these embodiments, a and b are each 2.

For certain embodiments, R₁₁ and R₁₂ are independently C₁₋₄ allyl or R₁₁ and R₁₂ together with the nitrogen atom to which they are attached form a 5- or 6-membered ring optionally containing —O—, —N(C₁₋₄ alkyl)-, or —S—. For certain embodiments, R₁₁ and R₁₂ are each methyl.

For certain embodiments, including any one of the above embodiments where X is present, X is selected from the group consisting of alkylene, alkenylene, alkynylene, arylene, heteroarylene, and heterocyclylene wherein the alkylene, alkenylene, and alkynylene groups can be optionally interrupted or terminated by arylene, heteroarylene or heterocyclylene and optionally interrupted by one or more —O— groups. For certain of these embodiments, X is C₂₋₆ alkylene. For certain of these embodiments, X is C₂₋₄ alkylene.

For certain embodiments, including any one of the above embodiments where Y is present, Y is selected from the group consisting of —O—, —S(O)₀₋₂—, —S(O)₂—N(R₈)—, —C(R₆)—, —O—C(R₆)—, —O—C(O)—O—, —N(R₈)-Q-, —O—C(R₆)—N(R₈)—, —C(R₆)—N(OR₉)—, —O—N(R₈)-Q-, —O—N═C(R₄)—, —C(═N—O—R₈)—, —CH(—N(—O—R₈)-Q-R₄)—,

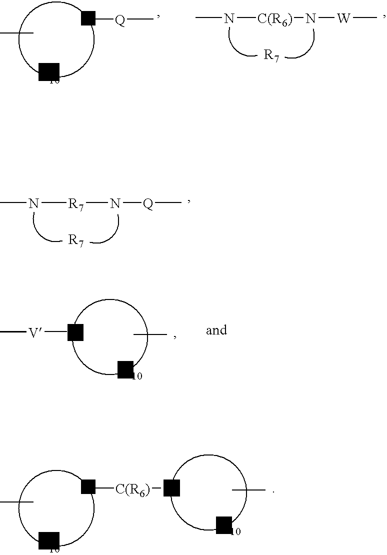

For certain of these embodiments, Y is —S(O)₂— or —N(R₈)-Q-.

For certain embodiments, including any one of the above embodiments where Y is present, Y is Yₐ.

For certain embodiments, including any one of the above embodiments where Yₐ is present, Yₐ is selected from the group consisting of —O—, —S(O)₀₋₂—, —S(O)₂—N(R₈)—, —N(R₈)-Q-, —O—C(R₆)—N(R₈)—, —C(R₆)—N(OR₉)—,

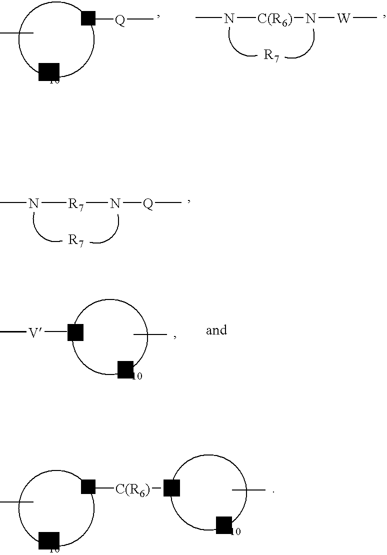

For certain of these embodiments, Yₐ is —S(O)₂— or —N(R₈)-Q-.

As used herein, the terms “alkyl”, “alkenyl”, “alkynyl”, and the prefix “alk-” are inclusive of both straight chain and branched chain groups and of cyclic groups, e.g., cycloalkyl and cycloalkenyl. Unless otherwise specified, these groups contain from 1 to 20 carbon atoms, with alkenyl groups containing from 2 to 20 carbon atoms, and alkynyl groups containing from 2 to 20 carbon atoms. In some embodiments, these groups have a total of up to 10 carbon atoms, up to 8 carbon atoms, up to 6 carbon atoms, or up to 4 carbon atoms. Cyclic groups can be monocyclic or polycyclic and preferably have from 3 to 10 ring carbon atoms. Exemplary cyclic groups include cyclopropyl, cyclopropylmethyl, cyclobutyl, cyclobutylmethyl, cyclopentyl, cyclopentylmethyl, cyclohexyl, cyclohexylmethyl, adamantyl, and substituted and unsubstituted bornyl, norbornyl, and norbornenyl.

Unless otherwise specified, “alkylene”, “alkenylene”, and “alkynylene” refer to a divalent form of the “alkyl”, “alkenyl”, and “alkynyl” groups defined above. The terms, “alkylenyl”, “alkenylenyl”, and “alkynylenyl” are used when “alkylene”, “alkenylene”, and “alkynylene”, respectively, are substituted. For example, an arylalkylenyl group comprises an alkylene moiety to which an aryl group is attached.

The term “haloalkyl” is inclusive of groups that are substituted by one or more halogen atoms, including perfluorinated groups. This is also true of other groups that include the prefix “halo-.” Examples of suitable haloalkyl groups are chloromethyl, trifluoromethyl, and the like.

The term “aryl” as used herein includes carbocyclic aromatic rings or ring systems. Examples of aryl groups include phenyl, naphthyl, biphenyl, fluorenyl and indenyl.

Unless otherwise indicated, the term “heteroatom” refers to the atoms O, S, or N.

The term “heteroaryl” includes aromatic rings or ring systems that contain at least one ring heteroatom (e.g., O, S, N). In some embodiments, the term “heteroaryl” includes a ring or ring system that contains 2-12 carbon atoms, 1-3 rings, 1-4 heteroatoms, and O, S, and N as the heteroatoms. Exemplary heteroaryl groups include furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, pyrazinyl, 1-oxidopyridyl, pyridazinyl, triazinyl, tetrazinyl, oxadiazolyl, thiadiazolyl, and so on.

The term “heterocyclyl” includes non-aromatic rings or ring systems that contain at least one ring heteroatom (e.g., O, S, N) and includes all of the fully saturated and partially unsaturated derivatives of the above mentioned heteroaryl groups. In some embodiments, the term “heterocyclyl” includes a ring or ring system that contains 2-12 carbon atoms, 1-3 rings, 1-4 heteroatoms, and O, S, and N as the heteroatoms. Exemplary heterocyclyl groups include pyrrolidinyl, tetrahydrofuranyl, morpholinyl, thiomorpholinyl, 1,1-dioxothiomorpholinyl, piperidinyl, piperazinyl, thiazolidinyl, imidazolidinyl, isothiazolidinyl, tetrahydropyranyl, quinuclidinyl, homopiperidinyl (azepanyl), 1,4-oxazepanyl, homopiperazinyl (diazepanyl), 1,3-dioxolanyl, aziridinyl, azetidinyl, dihydroisoquinolin-(1H)-yl, octahydroisoquinolin-(1H)-yl, dihydroquinolin-(2H)-yl, octahydroquinolin-(2H)-yl, dihydro-1H-imidazolyl, 3-azabicyclo[3.2.2]non-3-yl, and the like.

The term “heterocyclyl” includes bicyclic and tricyclic heterocyclic ring systems. Such ring systems include fused and/or bridged rings and spiro rings. Fused rings can include, in addition to a saturated or partially saturated ring, an aromatic ring, for example, a benzene ring. Spiro rings include two rings joined by one spiro atom and three rings joined by two spiro atoms.

When “heterocyclyl” contains a nitrogen atom, the point of attachment of the heterocyclyl group may be the nitrogen atom.

The terms “arylene”, “heteroarylene”, and “heterocyclylene” refer to a divalent form of the “aryl”, “heteroaryl”, and “heterocyclyl” groups defined above. The terms, “arylenyl”, “heteroarylenyl”, and “heterocyclylenyl” are used when “arylene”, “heteroarylene”, and “heterocyclylene”, respectively, are substituted. For example, an alkylarylenyl group comprises an arylene moiety to which an alkyl group is attached.

The term “fused 5 to 7 membered saturated ring” includes rings which are fully saturated except for the bond where the ring is fused.

When a group (or substituent or variable) is present more than once in any Formula described herein, each group (or substituent or variable) is independently selected, whether explicitly stated or not. For example, when more than one R′ group is present, then each R′ group is independently selected. In another example, in the formula O(—C(O)—R₂)₂, each R₂ group is independently selected.

The invention is inclusive of the compounds described herein in any of their pharmaceutically acceptable forms, including isomers (e.g., diastereomers and enantiomers), salts, solvates, polymorphs, prodrugs, and the like. In particular, if a compound is optically active, the invention specifically includes each of the compound's enantiomers as well as racemic mixtures of the enantiomers. It should be understood that the term “compound” includes any or all of such forms, whether explicitly stated or not (although at times, “salts” are explicitly stated).

### Preparation of the Compounds

More specific details of the reactions described herein are discussed in the context of the following schemes.

Some embodiments of the invention are described below in Reaction Schemes I through IX. For more detailed description of the individual reaction steps, see the EXAMPLES section below. The starting materials are generally available from commercial sources such as Aldrich Chemicals (Milwaukee, Wis., USA) or are readily prepared using methods well known to those skilled in the art (e.g., prepared by methods generally described in Louis F. Fieser and Mary Fieser, Reagents for Organic Synthesis, v. 1-19, Wiley, New York, (1967-1999 ed.); Alan R. Katritsky, Otto Meth-Cohn, Charles W. Rees, Comprehensive Organic Functional Group Transformations, v. 1-6, Pergamon Press, Oxford, England, (1995); Barry M. Trost and Ian Fleming, Comprehensive Organic Synthesis, v. 1-8, Pergamon Press, Oxford, England, (1991); or Beilsteins Handbuch der organischen Chemie, 4, Aufl. Ed. Springer-Verlag, Berlin, Germany, including supplements (also available via the Beilstein online database)).

Although specific starting materials and reagents are depicted in the reaction schemes and discussed below, other starting materials and reagents known to those skilled in the art can be substituted to provide a variety of derivatives and/or reaction conditions. In addition, many of the methods described below can be further elaborated in light of this disclosure using conventional methods well known to those skilled in the art.

In carrying out methods of the invention it may sometimes be necessary to protect a particular functionality while reacting other functional groups on an intermediate. The need for such protection will vary depending on the nature of the particular functional group and the conditions of the reaction step. Suitable amino protecting groups include acetyl, trifluoroacetyl, tert-butoxycarbonyl (Boc), benzyloxycarbonyl, and 9-fluorenylmethoxycarbonyl (Fmoc). Suitable hydroxy protecting groups include acetyl and silyl groups such as the tert-butyl dimethylsilyl group. For a general description of protecting groups and their use, see T. W. Greene and P. G. M. Wuts, Protective Groups in Organic Synthesis, 3^{rd} edition, John Wiley & Sons, New York, USA, 1999.

Conventional methods and techniques of separation and purification can be used to isolate compounds shown in the Reaction Schemes below. Such techniques may include, for example, all types of chromatography (high performance liquid chromatography (HPLC), column chromatography using common absorbents such as silica gel, and thin layer chromatography), recrystallization, and differential (i.e., liquid-liquid) extraction techniques.

Methods of the invention are shown in Reaction Scheme I, wherein R_{A}, R_{B}, R₁, R₂, E, and L are as defined above. In step (1) or (1a) of Reaction Scheme I, a 3-nitropyridine, 3-nitroquinoline, or 3-nitronaphthyridine of Formula V or II is reduced to a 3-aminopyridine, 3-aminoquinoline, or 3-aminonaphthyridine of Formula VI or III, respectively. The reduction can be carried out by a number of conventional methods. For example, the reaction can be carried out by hydrogenation using a heterogeneous hydrogenation catalyst such as platinum on carbon or Raney nickel. The hydrogenation can be conveniently carried out at room temperature in a Parr apparatus in a suitable solvent such as N,N-dimethylformamide (DMF). The reduction can also be carried out using nickel boride, prepared in situ from sodium borohydride and nickel(II) chloride. The nickel boride reduction is conveniently carried out by adding a solution of a compound of Formula V or II in a suitable solvent or solvent mixture such as dichloromethane/methanol to a mixture of excess sodium borohydride and catalytic or stoichiometric nickel(II) chloride in methanol. The reaction can be carried out at room temperature. Alternatively the reduction can be carried out using a one- or two-phase sodium dithionite reduction. The sodium dithionite reduction can be conveniently carried out using the conditions described by Park, K. K.; Oh, C. H.; and Joung, W. K.; Tetrahedron Lett., 34, pp. 7445-7446 (1993) by adding sodium dithionite to a compound of Formula V or II in a mixture of dichloromethane and, water at ambient temperature in the presence of potassium: carbonate and ethyl viologen dibromide, ethyl viologen diiodide, or 1,1′-di-n-octyl-4,4′-bipyridinium dibromide.

Many compounds of Formula V and II are known; others can be prepared by known methods. For quinolines and [1,5]naphthyridines of Formula V and II in which E is hydrogen and L is chloro, see U.S. Pat. Nos. 4,689,338 (Gerster) and 6,194,425 (Gerster et al.) and the references cited therein. Quinolines, tetrahydroquinolines, and pyridines of Formula II in which E and L are each chloro or —O—S(O)₂—R′, can be prepared from compounds of Formula V in which E is hydroxy; see, for example, U.S. Pat. Nos. 4,988,815 (Andre et al.), 5,395,937 (Nikolaides et al.), 5,352,784 (Nikolaides et al.), 5,446,153 (Lindstrom et al.), and 6,743,920. (Lindstrom et al.) and the references cited therein. For quinolines and naphthyridines of Formula V or II in which E is part of a tetrazolo ring and L is chloro or —O—S(O)₂—R′, see U.S. Pat. Nos. 6,194,425 (Gerster et al.) and 5,741,908 (Gerster et al.) and the references cited therein. Compounds of Formula II in which E and/or L is phenoxy can be prepared from compounds of Formula II in which E and/or L is chloro using the methods described in 6,743,920 (Lindstrom et al.). Compounds of Formula II in which E is —N(Bn)₂ can be prepared from compounds of Formula II in which E is —O—S(O)₂—R′ according to the methods described in U.S. Pat. No. 5,395,937 (Nikolaides et al.) and U.S. Pat. No. 5,352,784 (Nikolaides et al.).

Several compounds of Formula VI in which E is hydrogen are known compounds, including unsubstituted and substituted pyridines, quinolines, and naphthyridines of each isomeric variation. See, for example, U.S. Pat. No. 6,110,929 (Gerster et al.) and the references cited therein. Also, some compounds of Formula III are known. For example, 3-amino-4-chloroquinoline, 3-amino-4,5-dichloroquinoline, and 3-amino-4,7-dichloroquinoline have been prepared by Surrey et al., Journal of the American Chemical Society, 73, pp. 2413-2416 (1951).

In step (2) or (3a) of Reaction Scheme I, a 3-aminopyridine, 3-aminoquinoline, or 3-aminonaphthyridine of Formula VI or III is reacted with a carboxylic acid or an equivalent thereof to provide an amide-substituted compound of Formula VII or IV, respectively. Suitable equivalents to carboxylic acids include acid anhydrides and acid halides. The selection of the carboxylic acid equivalent is determined by the desired substituent at R₂. For example, the use of butyryl chloride provides a compound in which R₂ is a propyl group; the use of ethoxyacetyl chloride provides a compound in which R₂ is an ethoxymethyl group. The reaction can be conveniently carried out by combining an acid halide of Formula R₂C(O)Cl or R₂C(O)Br with a compound of Formula VI or III in a suitable solvent such as dichloromethane, acetonitrile, or 1,2-dichloroethane optionally in the presence of a tertiary amine such as triethylamine, pyridine, or 4-dimethylaminopyridine (DMAP). The reaction can be run at a reduced temperature, for example, 0° C., at room temperature, or at an elevated temperature, such as 40° C. to 90° C.—For compounds wherein R₂ is hydrogen, a compound of Formula VI or III can be reacted with a formulating agent such as, for example, diethoxymethyl acetate or acetic formic anhydride. Some compounds of Formula VII are known; see, for example, U.S. Pat. No. 6,110,929 (Gerster et al.).

In step (3) of Reaction Scheme I, the hydroxy group in a compound of Formula VII is converted to a leaving group using conventional activation methods to provide a compound of Formula IV. For example, conversion of the hydroxy group to a chloro group can be conveniently carried out by combining a compound of Formula VII with phosphorus(III) oxychloride. The chlorination reaction can be carried out neat or in a suitable solvent such as N,N-dimethylformamide (DMF), dichloromethane, acetonitrile, 1-methyl-2-pyrrolidinone (NMP), and 1,2-dichloroethane. The reaction can be carried out at room temperature or at an elevated temperature up to the reflux temperature, for example, at a temperature of 25° C. to 120° C. Other examples of chlorinating agents include, for example, thionyl chloride, phosgene, oxalyl chloride, and phosphorus pentachloride. Other halogenating agents include phosphorus(III) oxybromide, phosphorus pentabromide, diphenylphosphinic chloride, and triphenylphosphine in the presence of bromine. The hydroxy group in a compound of Formula VII can also be converted to a sulfonate ester by reaction with, for example, a sulfonyl halide or sulfonic anhydride. Suitable sulfonating agents include methanesulfonyl chloride, methanesulfonic anhydride, trifluoromethanesulfonyl chloride, trifluoromethanesulfonic anhydride, N-phenylbis(trifluoromethanesulfonimide), benzenesulfonyl chloride, benzenesulfonic anhydride, p-bromobenzenesulfonyl chloride, p-bromobenzenesulfonic anhydride, p-toluenesulfonyl chloride, p-toluenesulfonic anhydride, 2-nitrobenenesulfonyl chloride and 4-nitrobenzenesulfonyl chloride. The reaction with a sulfonating agent is typically carried out in the presence of a base. Preferably the base is a tertiary amine such as triethylamine. The reaction can be carried out in a suitable solvent such as dichloromethane, 1,2-dichloroethane, acetonitrile, tetrahydrofuran (THF), DMF, and NMP. The reaction may also be carried out in pyridine, which can be used as both the base and the solvent for the reaction. The reaction may be carried out at room temperature or an elevated temperature, such as the reflux temperature of the solvent. Preferably the reaction temperature is about room temperature to no higher than 90° C. These methods described for step (3) of Reaction Scheme I can also be used to convert a 3-aminopyridine, 3-aminoquinoline, or 3-aminonaphthyridine of Formula VI to a compound of Formula III, as shown in step (2a) of Reaction Scheme I.

In step (4) of Reaction Scheme I, an amide of Formula IV is reacted with an amine of formula R₁NH₂, or a suitable salt thereof, to provide a 1H-imidazo compound of Formula I. The reaction may be carried out neat at an elevated temperature such as the temperature required to melt the mixture. The reaction may also be carried out in a suitable solvent at an elevated temperature. Suitable solvents include alcohols such as methanol, ethanol, trifluoroethanol, isopropanol, and tert-butanol; water; acetonitrile; NMP; and toluene. Preferred solvents include trifluoroethanol, isopropanol, and tert-butanol. Preferably, the reaction temperature is not lower than 80° C. and not higher than 200° C. More preferably, the reaction temperature is not higher than 180° C. More preferably, the reaction temperature is 110° C. to 165° C. Optionally, a base may be used in the reaction. Suitable bases include triethylamine. Optionally, a catalyst such as pyridine hydrochloride, pyridinium p-toluenesulfonate, or p-toluenesulfonic acid can be added. For some amines of formula R₁NH₂ under certain conditions, an uncyclized 3-amido-4-amino intermediate may be isolated. The intermediate can then be cyclized in a subsequent step by heating in a solvent such as toluene, optionally in the presence of a catalyst such as pyridine hydrochloride or pyridinium p-toluenesulfonate. The cyclization may be carried out at an elevated temperature, such as the reflux temperature of the solvent.

Numerous primary amines of formula R₁NH₂, or salts thereof, suitable for this reaction are commercially available; others can be prepared by known methods. See, for example, the methods in U.S. Pat. Nos. 6,451,810 (Coleman et al.), 6,660,747 (Crooks et al.), 6,683,088 (Crooks et al.), and 6,656,938 (Crooks et al.); U.S. Patent Application Publication No. 2004/0147543 (Hays et al.); and International Publication No. WO2005/051317 (Krepski et al.).

Some amines of the formula R₁NH₂ can be made according to the following method. For some embodiments, R₁ is a 1-hydroxycycloalkylmethyl group. The corresponding amine of formula R₁NH₂ can be, prepared by combining a cyclic ketone, such as cyclopentanone or cyclobutanone with excess nitromethane in a suitable solvent such as ethanol or methanol in the presence of a catalytic amount of base such as sodium ethoxide or sodium hydroxide and reducing the resultant nitromethyl-substituted compound using conventional heterogeneous hydrogenation conditions. The hydrogenation is typically carried out in the presence of a catalyst such as palladium hydroxide on carbon, palladium on carbon, or Raney nickel in a suitable solvent such as ethanol. Both the reaction with nitromethane and the reduction can be carried out at room temperature. A wide variety of cyclic ketones can be obtained from commercial sources; others can be synthesized using known synthetic methods.

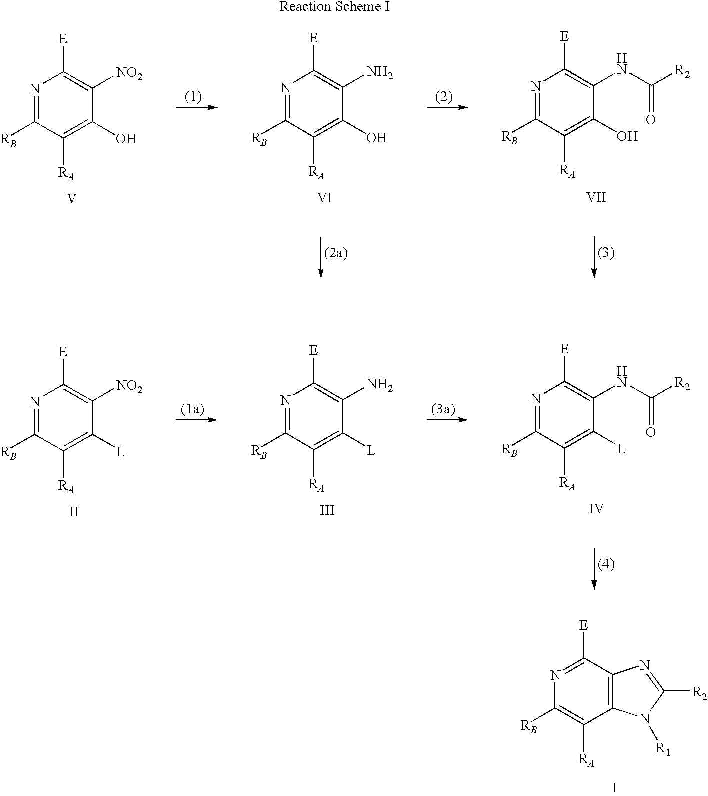

A compound of Formula I can be converted to a compound of Formula X using a variety of methods, depending on the identity of E. Examples of these methods are shown in Reaction Schemes II through V.

For certain embodiments, the amination of a compound of Formula I₁ is shown in Reaction Scheme II, wherein E₁ is a halogen, phenoxy, or —O—S(O)₂—R′, and R_{A}, R_{B}, R₁, R₂ are as defined above. Step (1) of Reaction Scheme II can be used to convert a compound of Formula I-4, wherein E is hydroxy, to a compound of Formula I₁. Any one of the methods described in step (3) and step (2a) of Reaction Scheme I can be used. The amination in step (2) of Reaction Scheme II can be conveniently carried out by heating a combination of a compound of Formula I₁ and a solution of ammonia in a suitable solvent such as methanol. The amination may also be carried out by using ammonium acetate or ammonium hydroxide in combination with a compound of Formula I₁ and heating. The amination is preferably carried out at a temperature not lower than 100° C., preferably not lower than 125° C., more preferably not lower than 140° C. The reaction is preferably carried out at a temperature not higher than 200° C., more preferably not higher than 170° C.

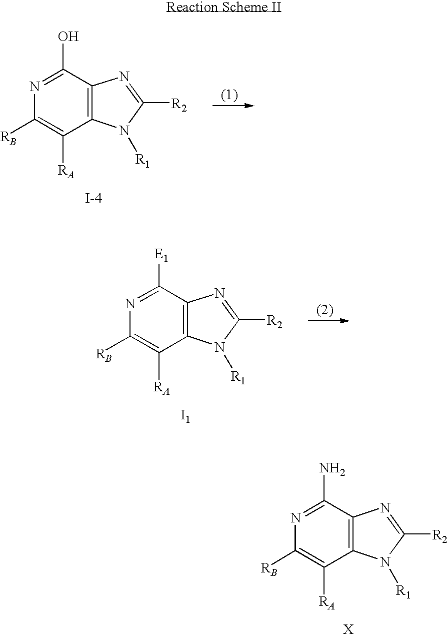

Alternatively, a compound of Formula I, can be converted in two steps to a compound of Formula X as shown in Reaction Scheme III, wherein R_{A}, R_{B}, R₁, R₂, E₁, and Bn are as defined above. Step (1) of Reaction Scheme III can be used to displace the E₁ group in a compound of Formula II with an amine of Formula HN(Bn)₂ to provide a compound of Formula I-6. The displacement can be conveniently carried out by combining an amine of formula HN(Bn)₂ and a compound of Formula I₁ in a suitable solvent such as toluene or xylenes in the presence of a base such as triethylamine and heating at an elevated temperature such as the reflux temperature of the solvent. In step (2) of Reaction Scheme III, the protecting groups are removed from the 4-amine of a compound of Formula I-6 to provide a compound of Formula X. For certain embodiments, the deprotection can be conveniently carried out on a Parr apparatus under hydrogenolysis conditions using a suitable heterogeneous catalyst such as palladium on carbon in a solvent such as ethanol. Alternatively, when Bn is p-methoxybenzyl, step (2) may carried out by combining trifluoroacetic acid and a compound of Formula I-6 and stirring at room temperature or heating at an elevated temperature such as 50° C. to 70° C.

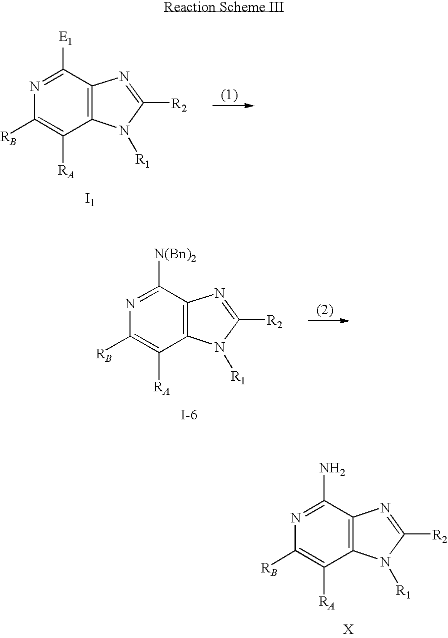

For certain embodiments, a compound of Formula I wherein E is hydrogen can be converted in to a compound of Formula X₂ by oxidation and amination as shown in Reaction Scheme IV, wherein R_{A2} and R_{B2} taken together form a fused benzene ring or a fused pyridine ring wherein the benzene ring or pyridine ring is unsubstituted or substituted by one R group, or substituted by one R₃ group, or substituted by one R group and one R₃ group, and R₁ and R₂ are as defined above. In step (1) of Reaction Scheme IV, a compound of Formula I-2₂ is oxidized to a 5N-oxide of Formula XX₂ using a conventional oxidizing agent capable of forming N-oxides. The reaction is conveniently carried out by combining 3-chloroperoxybenzoic acid with a compound of Formula I-2₂ in a suitable solvent such as dichloromethane or chloroform. The reaction can be carried out at room temperature. Alternatively, other peracids such as peracetic acid can be used as the oxidizing agent. The reaction with peracetic acid can be carried out in a suitable solvent such as ethanol at an elevated temperature such as 50° C. to 60° C. The 5N-oxide of Formula XX₂ is then aminated in step (2) of Reaction Scheme IV to provide a compound of Formula X₂. The amination can be carried out by the activation of the 5N-oxide by conversion to an ester and then reacting the ester with an aminating agent. Suitable activating agents include alkyl- or arylsulfonyl chlorides such as benzenesulfonyl chloride, methanesulfonyl chloride, or p-toluenesulfonyl chloride. Suitable aminating agents include ammonia, in the form of ammonium hydroxide, for example, and ammonium salts such as ammonium carbonate, ammonium bicarbonate, and ammonium phosphate. The reaction is conveniently carried out by adding p-toluenesulfonyl chloride to a mixture of ammonium hydroxide and a solution of the 5N-oxide in a suitable solvent such as dichloromethane or chloroform. The reaction can be carried out at room temperature. The oxidation and amination steps may be carried out as a one-pot procedure without isolating the 5N-oxide of Formula XX₂. Alternatively, the 5N-oxide can be treated in step (2) with an isocyanate wherein the isocyanato group is bonded to a hydrolytically active functional group; subsequent hydrolysis of the resulting intermediate provides a compound of Formula X₂. The reaction can be conveniently carried out in two steps by (i) combining an isocyanate such as trichloroacetyl isocyanate and a solution of the 5N-oxide in a solvent such as dichloromethane and stirring at room temperature to provide an isolable amide intermediate. In step (ii), a solution of the intermediate in methanol can be treated with a base such as sodium methoxide at room temperature.

Alternatively, a 5N-oxide of Formula XX can be converted to a compound of Formula I₁ wherein E₁ is chloro using one of the methods described in step (3) of Reaction Scheme I. The resulting 4-chloro compound can then be aminated according to the methods described in Reaction Scheme II.

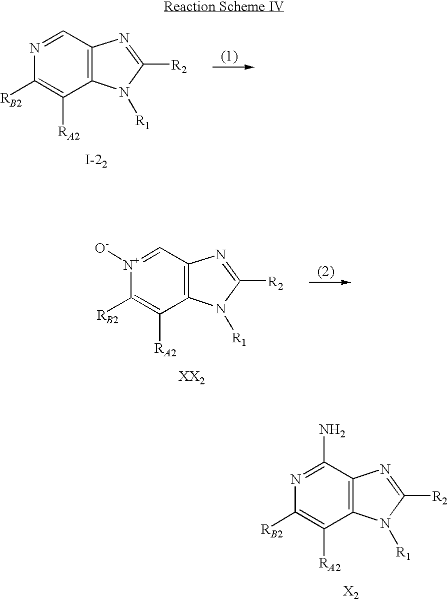

For embodiments wherein E is joined with the pyridine nitrogen atom in a compound of Formula I to form a fused tetrazolo ring, the tetrazolo ring can be removed to form a compound of Formula X as shown in Reaction Scheme V, wherein R_{A}, R_{B}, R₁, and R₂ are as defined above, and Ph is phenyl. In step (1) of Reaction Scheme V, a compound of Formula I-1 is combined with triphenylphosphine to form an N-triphenylphosphinyl intermediate of Formula XXI. The reaction with triphenylphosphine can be run in a suitable solvent such as toluene or 1,2-dichlorobenzene under an atmosphere of nitrogen with heating, for example at the reflux temperature.

In step (2) of Reaction Scheme V, an N-triphenylphosphinyl intermediate of Formula XXI is hydrolyzed to provide a compound of Formula X. The hydrolysis can be carried out by general methods well known to those skilled in the art, for example, by heating in a lower alkanol or an alkanol/water solution in the presence of an acid such as trifluoroacetic acid, acetic acid, or hydrochloric acid. A compound of Formula X may also be obtained through an alternative route as shown in step (1a) of Reaction Scheme V. In step (1a), the tetrazolo ring is reductively removed from a compound of Formula I-1 to provide a compound of Formula X. The reaction can be carried out by reacting the compound of Formula I-1 with hydrogen in the presence of a catalyst and an acid. The hydrogenation can be conveniently run at ambient temperature on a Parr apparatus with a suitable catalyst, such as platinum IV oxide, and a suitable acid, such as trifluoroacetic acid or hydrochloric acid. The reaction can optionally be carried out in the presence of a solvent such as, for example, ethanol. If step (1a) is used, a compound of Formula I-1 in which R_{A} and R_{B} taken together form a fused benzene ring or a fused pyridine ring may be converted to a compound of Formula X in which R_{A} and R_{B} taken together form a fused 5 to 7-membered saturated ring optionally containing one nitrogen atom. One of skill in the art would understand that other groups susceptible to reduction, such as alkenyl, alkynyl, and aryl groups, would be reduced in step (1a).

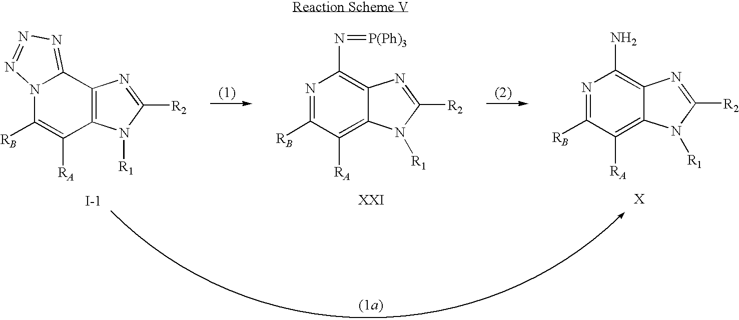

For certain embodiments, compounds of Formula X_{b} can be reduced according to Reaction Scheme VI, wherein R_{A3} and R_{B3} taken together form a fused benzene ring or a fused pyridine ring wherein the benzene ring or pyridine ring is unsubstituted or substituted by one R_{b} group, or substituted by one R_{3b} group, or substituted by one R_{b} group and one R_{3b} group; R_{A4} and R_{B4} taken together form a fused 5 to 7-membered saturated ring optionally containing one nitrogen atom, wherein the fused ring is unsubstituted or substituted by one or more R_{b} groups; and R_{1b}, R_{2b}, R_{3b}, and R_{b} are subsets of R₁, R₂, R₃, and R as defined above that do not include those substituents that one skilled in the art would recognize as being susceptible to reduction under the acidic hydrogenation conditions of the reaction. These susceptible groups include, for example, alkenyl, alkynyl, and aryl groups and groups bearing nitro substituents.

As shown in Reaction Scheme VI, an 1H-imidazo[4,5-c]quinolin-4-amine or 1H-imidazo[4,5-c][1,5]naphthyridin-4-amine of Formula X_{b} can be reduced to a 6,7,8,9-tetrahydroquinoline or tetrahydronaphthyridine of Formula X_{c}. Compounds of Formula X_{b} can be prepared according to the methods described in Reaction Schemes II, III, IV, or V. The reaction is conveniently carried out under hetereogeneous hydrogenation conditions by adding platinum (IV) oxide to a solution of the compound of Formula X_{b} in trifluoroacetic acid and placing the reaction under hydrogen pressure. The reaction can be carried out on a Parr apparatus at ambient temperature.

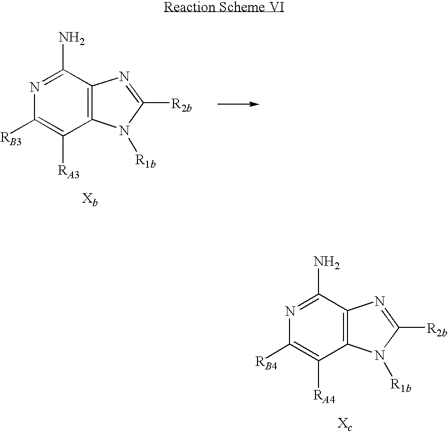

For some embodiments, compounds shown in Reaction Schemes I through VI can be further elaborated using conventional synthetic methods. Amines of formula R₁NH₂, used in step (4) of Reaction Scheme I, may contain a protected functional group, such as a tert-butoxycarbonyl-protected amino group. For example, protected diamines of Formula Boc-N(R₈)—X—NH₂,

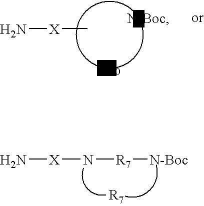

are commercially available or can be prepared by known methods; see, for example, U.S. Pat. Nos. 6,660,747 (Crooks et al.), 6,683,088 (Crooks et al.), and 6,656,938 (Crooks et al.) and Carceller, E. et al., J. Med. Chem., 39, pp. 487-493 (1996). The protecting group may be removed after the cyclization step shown in step (4) of Reaction Scheme I or after the steps shown in Reaction Schemes II through VI to reveal, for example, an amino substituent on the R₁ group. An amino group introduced in this manner can react with an acid chloride of Formula R₄C(O)Cl, a sulfonyl chloride of Formula R₄S(O)₂Cl, a sulfonic anhydride of Formula (R₄S(O)₂)₂O, or an isocyanate of Formula R₄N═C═O to provide a compound of Formula X in which R₁ is —X—N(R₈)-Q-R₄,

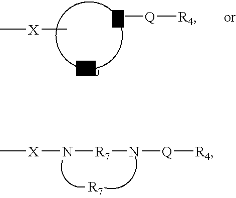

where X, R₄, R₇, R₈, and R₁₀ are as defined above and Q is —C(O)—, —SO₂—, or —C(O)—NH—. Numerous acid chlorides, sulfonyl chlorides, sulfonic anhydrides, and isocyanates are commercially available; others can be readily prepared using known synthetic methods. The reaction can be conveniently carried out by combining the acid chloride, sulfonyl chloride, sulfonic anhydride, or isocyanate and a solution of an amino-substituted compound, and a base such as triethylamine in a suitable solvent such as dichloromethane. The reaction can be carried out at room temperature.

Amines of formula R₁NH₂ can also contain other protected functional groups, such as ketal-protected ketones. For example, 2,2-dimethyl-3-(2-methyl-1,3-dioxolan-2-yl)propylamine, prepared in Example 22 of International Publication No. WO2005/051317 (Krepski et al.), can be used in step (4) of Reaction Scheme I. The ketal protecting group can later be removed by conventional methods to provide a compound of Formula I or X in which R₁ is 2,2-dimethyl-4-oxopentyl.

Amino alcohols of formula H₂N—X—OH can be used in step (4) of Reaction Scheme I, and the hydroxy functional group can be converted in subsequent steps to a compound of Formula I or X having an —X—S(O)₀₋₂—R₄, —X—S(O)₂—N(R₈)—R₄, —X—O—N(R)-Q-R₄, —X—O—N═C(R₄)—R₄, —X—CH(—N(—O—R₈)-Q-R₄)—R₄ group at the R₁ position using methods described in U.S. Pat. No. 6,664,264 (Dellaria et al.) and International Publication Nos. WO2005/066169 (Bonk and Dellaria), WO2005/018551 (Kshirsagar et al.), WO2005/018556 (Kshirsagar et al.), and WO2005/051324 (Krepski et al.), respectively.

The amine used in step (1) may be tert-butyl carbazate, and the resulting compound of Formula I or subsequently converted compound of Formula X wherein R₁ is a Boc-protected amino group can be deprotected to provide a 1-amino compound or a salt (for example, hydrochloride salt) thereof. The deprotection can be carried out by heating at reflux a solution of a compound of Formula I or X in ethanolic hydrogen chloride. The resulting compound of Formula I or X wherein R₁ is an amino group can treated with a ketone, aldehyde, or corresponding ketal or acetal thereof, under acidic conditions. For example, a ketone can be added to a solution of the hydrochloride salt of a compound of Formula I or X in which R₁ is an amino group in a suitable solvent such as isopropanol or acetonitrile in the presence of an acid such as pyridinium p-toluene sulfonate or acetic acid, or an acid resin, for example, DOWEX W50-X1 acid resin. The reaction can be performed at an elevated temperature. The resulting imine can be reduced to provide a compound of Formula I or X in which R₁ is —N(R₁′)-Q-R₄, wherein Q is a bond The reduction can be carried out at room temperature with sodium borohydride in a suitable solvent, for example, methanol. A tert-butyl hydrazinecarboxylate can also be manipulated in subsequent steps using the methods of U.S. Patent Application Publication No. 2005/0054640 to provide other compounds of Formula I or X, wherein R₁ is —N(R₁′)-Q-R₄, —N(R₁′)—X₁—Y₁—R₄, or —N(R₁′)—X₁—R_{5b}.

Other transformations at the R₁ position can be made. See, for example, U.S. Pat. Nos. 5,389,640 (Gerster et al.), 6,331,539 (Crooks et al.), 6,451,810 (Coleman et al.), 6,541,485 (Crooks et al.), 6,660,747 (Crooks et al.), 6,670,372 (Charles et al.), 6,683,088 (Crooks et al.), 6,656,938 (Crooks et al.), 6,664,264 (Dellaria et al.), 6,677,349 (Griesgraber), and 6,664,260 (Charles et al.).

For some embodiments, synthetic transformations can be made at the R₂ position in a compound of Formula I or X, if, for example, the carboxylic equivalent used in step (2) or (3a) of Reaction Scheme I contains a protected or unprotected hydroxy group or a protected amino group. Some carboxylic acid equivalents of this type are commercially available; others can be prepared by known synthetic methods. A protected hydroxy or amino group installed at the R₂ position can be deprotected by a variety of methods well known to one of skill in the art. For example, a hydroxyalkylenyl group is conveniently introduced at the R₂ position by the dealkylation of a methoxy- or ethoxyalkylenyl group, which can be installed by using a methoxy- or ethoxy-substituted carboxylic acid equivalent in step (2) or (3a) of Reaction Scheme I. The dealkylation can be carried out by treating a compound of Formula I or Formula X wherein R₂ is an alkoxyalkylenyl group with boron tribromide in a suitable solvent such as dichloromethane at a sub-ambient temperature such as 0° C. The resulting hydroxy group may then be oxidized to an aldehyde or carboxylic acid or converted to a leaving group such as, for example, a chloro group using thionyl chloride or a trifluoromethanesulfonate group using trifluoromethanesulfonic anhydride. The resulting leaving group can then be displaced by a variety of nucleophiles. Sodium azide can be used as the nucleophile to install an azide group, which can then be reduced to an amino group using heterogeneous hydrogenation conditions. An amino group at the R₂ position can be converted to an amide, sulfonamide, sulfamide, or urea using conventional methods. A leaving group at R₂, such as a chloro or trifluoromethanesulfonate group, can also be displaced with a secondary amine, a substituted phenol, or a mercaptan in the presence of a base such as potassium carbonate, For examples of these and other methods used to install a variety of groups at the R₂ position, see U.S. Pat. No. 5,389,640 (Gerster et al.). These synthetic transformations may conveniently be carried out as the last steps in the synthesis or prior to the steps shown in Reaction Schemes II through VI.

1H-Imidazo[4,5-c]quinolines of Formulas II and V wherein the quinoline ring is substituted by a bromo, a benzyloxy, or a methoxy substituent and E is hydrogen are known or can be made according to the methods described in U.S. Patent Application Publication No. 2004/0147543 (Hays et al.) and International Publication No. WO2005/020999 (Lindstrom et al.). These compounds can be subjected to the methods of Reaction Scheme I to provide 1H-imidazo[4,5-c]quinolines of Formula I-2₂, which can then be oxidized and aminated according to the methods of Reaction Scheme IV. Compounds in which the quinoline ring is substituted by a benzyloxy or methoxy group can then be converted to a hydroxy-substituted 1H-imidazo[4,5-c]quinolin-4-amine of Formula XXII, shown below in Reaction Scheme VII. The demethylation of a methoxy-substituted compound can be carried out with boron tribromide as described in the previous paragraph. Alternatively, the demethylation can be carried out by heating the methoxy-substituted compound with anhydrous pyridinium chloride at an elevated temperature, such as 210° C. Removal of a benzyloxy group can be carried out as described in International Publication No. WO2005/020999 (Lindstrom et al.).

Further synthetic elaboration of 1H-imidazo[4,5-c]quinolin-4-amines can then be carried out as shown in Reaction Scheme VII, wherein R₃C is -R₄, -X-R₄, -X-Y-R₄, -X-Y-X-Y-R₄, or -X-R₅; R_{3d} is —O—R₄, —O—X—R₄, —O—X—Y—R₄, —O—X—Y—X—Y—R₄, or —O—X—R₅; R_{d} is selected from the group consisting of halogen, alkyl, alkenyl, trifluoromethyl, and dialkylamino; n is 0 or 1; and R₁ and R₂ are defined as above. In step (1) of Reaction Scheme VII, the hydroxy group of a 1H-imidazo[4,5-c]quinolin-4-amine of Formula XXII is activated by conversion to a trifluoromethanesulfonate (triflate) group according to any one of the methods described in step (2a) or (3) of Reaction Scheme I to provide a compound of Formula XXIII.

Step (2) of Reaction Scheme VII can be carried out using known palladium-catalyzed coupling reactions such as the Suzuki coupling, Heck reaction, the Stille coupling, and the Sonogashira coupling according to any of the methods described in U.S. Patent Application Publication No. 2004/0147543 (Hays et al.) to provide a compound of Formula XXIV. Compounds in which a bromo substituent is used instead of a triflate group in a compound of Formula XXIII can alternatively be used in this transformation. The Suzuki coupling is carried out with an aryl or vinyl boronic acid, an anhydride thereof, or a boronic acid ester. The Heck reaction is carried out with vinyl-substituted compound. Sonogashira and Stille coupling reactions can be carried out with alkynes, and any of the unsaturated compounds prepared by theses couplings can undergo reduction of an alkenylene or alkynylene group.

In step (1a) of Reaction Scheme VII, a hydroxy-substituted 1H-imidazo[4,5-c]quinolin-4-amine of Formula XXII is converted to a compound of Formula XXV using a Williamson-type ether synthesis. The methods described in International Publication Nos. WO2005/020999 (Lindstrom et al.) and WO2005/032484 (Lindstrom et al.) can be used.

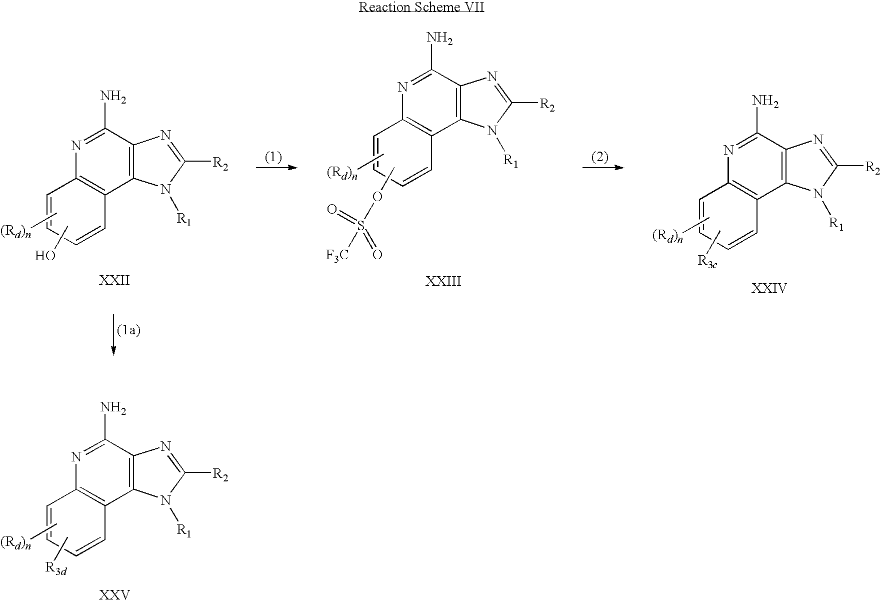

For certain embodiments, methods of the invention are shown Reaction Scheme VIII, wherein R_{A}, R_{B}, R₁, R₁₁, R₁₂, E, and L are as defined above. In step (1) of Reaction Scheme VIII, a 3-aminopyridine, 3-aminoquinoline, or 3-aminonaphthyridine of Formula VI is converted to an imidoformamide of Formula VIII. The reaction can be carried out by combining a 3-aminopyridine, 3-aminoquinoline, or 3-aminonaphthyridine of Formula VI or a suitable salt thereof with a halogenating agent or sulfonating agent described in step (3) or (2a) of Reaction Scheme I in the presence of a formamide of formula HC(O)—N(R₁₁)R₁₂. Several formamides of formula HC(O)—N(R₁₁)R₁₂ are commercially available, such as, for example, DMF, N,N-diethylformamide, and 1-formylpiperidine. Other formamides of this formula can be prepared by known methods; for example, an amine can be combined with a mixture of formic acid and acetic anhydride in a suitable solvent such as THF, acetone, acetonitrile, ethyl acetate, tert-butyl methyl ether, DMF, NMP, dichloromethane, toluene, xylenes, methanol, and ethanol. The reaction can be carried out at room temperature, below room temperature at a temperature of about 0° C. up to room temperature, or at an elevated temperature up to the reflux temperature of the solvent. Step (1) of Reaction Scheme VIII may be carried out in a solvent such as dichloromethane, 1,2-dichloroethane, acetonitrile, THF, toluene, and NMP, or in some embodiments, the reaction can be carried out in excess DMF. The reaction may be carried out at room temperature, below room temperature such as a temperature not lower than 0° C., or at an elevated temperature, such as a temperature not higher than the reflux temperature of the solvent. For some embodiments, the reaction temperature is not higher than 40° C. For some embodiments, step (1) of Reaction Scheme VIII can be carried out by combining a compound of Formula VI with phosphorus(III) oxychloride in excess DMF at room temperature or at an elevated temperature such as a temperature not higher than 150° C. For some embodiments, DMF may be used as the solvent. For some embodiments, the reaction temperature is 15° C. to 30° C.

For some embodiments, the conversion of a 3-aminopyridine, 3-aminoquinoline, or 3-aminonaphthyridine of Formula VI to a imidoformamide of Formula VIII can be carried out in two steps, such as steps (1a) and (2a) or (1b) and (2b) of Reaction-Scheme VIII. Steps (1b) and (2a) can be carried out according to the methods described in step (3) or (2a) of Reaction Scheme I in a solvent other than DMF. Steps (1a) and (2b) can be carried out as described in step (1) of Reaction Scheme VIII but in the absence of a halogenating or sulfonating agent.

In step (3) of Reaction Scheme VIII, an imidoformamide of Formula VIII is reacted with an amine of formula R₁NH₂, or a suitable salt thereof, to provide a 1H-imidazo compound of Formula I-H. The reaction may be carried out neat at an elevated temperature such as the temperature required to melt the mixture. The reaction may also be carried out in a suitable solvent at room temperature or at an elevated temperature. Suitable solvents include alcohols such as methanol, ethanol, trifluoroethanol, isopropanol, and tert-butanol; water; acetonitrile; NMP; toluene, and tetrahydrofuran. Preferred solvents include trifluoroethanol, isopropanol, tert-butanol, and acetonitrile. Preferably, the reaction temperature is not higher than 250° C. The reaction may be carried out at a temperature not higher than 200° C. or at a temperature not higher than 180° C. Optionally, a base may be used in the reaction. Suitable bases include triethylamine. Optionally, a catalyst such as pyridine hydrochloride, pyridinium p-toluenesulfonate, or p-toluenesulfonic acid can be added.

Under certain conditions, an imidoformamide intermediate of Formula XI,

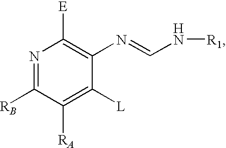

may be formed during step (3) of Reaction Scheme VIII. For some embodiments, the intermediate of Formula XI is isolated from the reaction mixture. The intermediate can then be cyclized in a subsequent step. The cyclization may be carried out by heating optionally in a solvent such as those described in the previous paragraph and optionally in the presence of a catalyst such as pyridine hydrochloride, pyridinium p-toluenesulfonate, or p-toluenesulfonic acid. Preferably, the cyclization reaction temperature is not higher than 250° C. The cyclization may be carried out at a temperature not higher than 200° C. or at a temperature not higher than 180° C.

For some embodiments, steps (1) and (3) of Reaction Scheme VIII are carried out as a one-pot procedure without isolating a compound of Formula VIII. The method may be carried out by adding an amine of formula R₁NH₂, or a suitable salt thereof, directly to the reaction mixture from step (1) of Reaction Scheme VIII. Optionally, the reaction mixture may be filtered prior to the addition of the amine of formula R₁NH₂. The resulting mixture can then be subjected to the conditions of step (3) to provide a compound of Formula I-H.

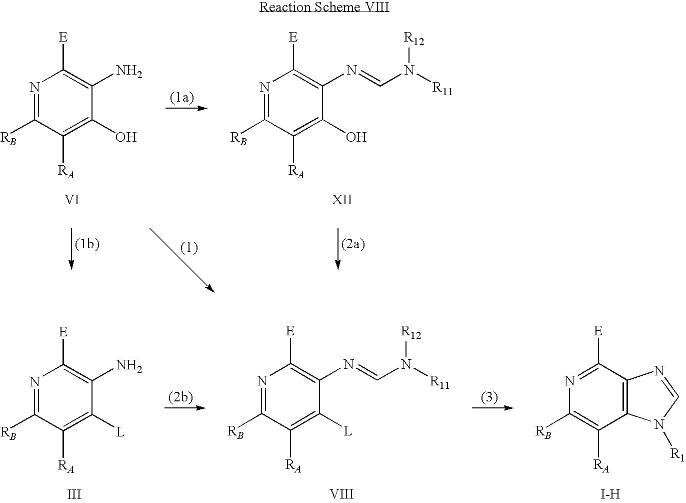

For certain embodiments, methods of the invention are shown Reaction Scheme IX, wherein R_{A}, R_{B}, R₁, E, and L are as defined above. In step (1) of Reaction Scheme IX, a 3-aminopyridine, 3-aminoquinoline, or 3-aminonaphthyridine of Formula VI is converted to an imidoformamide of Formula XI. Step (1) of Reaction Scheme IX may be carried out under the conditions described for step (1) of Reaction Scheme VIII using a formamide of formula HC(O)—NHR₁, instead of HC(O)—N(R₁₁)R₁₂. Some formamides of formula HC(O)—NHR₁, are commercially available. Others can be prepared by known methods; for example, an amine of formula R₁NH₂ can be combined with a mixture of formic acid and acetic anhydride using any of the solvents and conditions described in step (1) of Reaction Scheme VIII in connection with the preparation of HC(O)—N(R₁₁)R₁₂. Alternatively, an amine of formula R₁NH₂ can be combined with another formulating agent such as methyl formate, formamide, and chloroform in the presence of sodium hydroxide under to conditions known to one of skill in the art. See, for example, J. Org. Chem., 23, p. 1032 (1958), J. Am. Chem. Soc. 78, p. 2467 (1956), J. Chem. Soc., p. 858 (1957), J. Am. Chem. Soc. 74, p. 5619 (1952), and Tetrahedron Lett., 7, p. 5 (1959). The compound of Formula XI may be isolated from the reaction mixture prior to step (2) of Reaction Scheme IX, or steps (1) and (2) may be carried out without isolating a compound of Formula XI.

If an imidoformamide of Formula XI is isolated, step (2) of Reaction Scheme IX can be used to cyclize a compound of Formula XI to a compound of Formula I-H. The reaction may be carried out neat at an elevated temperature such as the temperature required to melt the compound of Formula XI. The reaction may also be carried out in a suitable solvent at room temperature or at an elevated temperature. Suitable solvents include alcohols such as methanol, ethanol, trifluoroethanol, isopropanol, and tert-butanol; water; acetonitrile; NMP; and toluene. Preferred solvents include trifluoroethanol, isopropanol, tert-butanol, and acetonitrile. Preferably, the cyclization reaction temperature is not higher than 250° C. The cyclization may be carried out at a temperature not higher than 200° C. or at a temperature not higher than 180° C. Optionally, a base may be used in the reaction. Suitable bases include triethylamine. Optionally, a catalyst such as pyridine hydrochloride, pyridinium p-toluenesulfonate, or p-toluenesulfonic acid can be added.

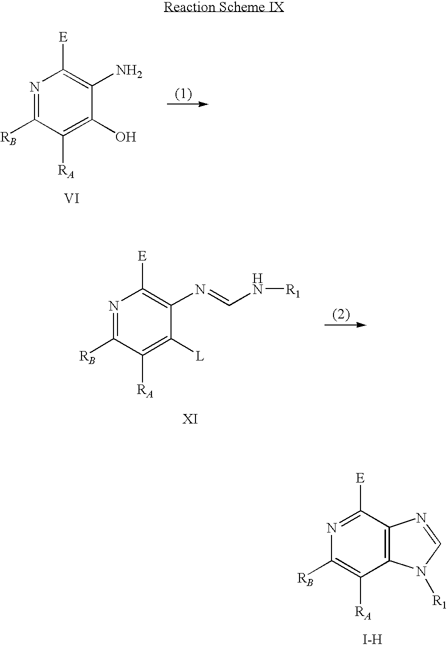

A compound of Formula I-H can be converted to a compound of Formula X, wherein R₂ is hydrogen, using a variety of methods, depending on the identity of E. Examples of these methods are shown in Reaction Schemes II through V, wherein R₂ is hydrogen. The synthetic methods of Reaction Schemes VI and VII can also be carried out using starting materials X_{b} and XXII wherein R_{2b} and R₂, respectively, are hydrogen.

### EXAMPLES

Objects and advantages of this invention are further illustrated by the following examples, but the particular materials and amounts thereof recited in these examples, as well as other conditions and details, should not be construed to unduly limit this invention.

### Example 1

### Preparation of 1-[4-Amino-2-(ethoxymethyl)-1H-imidazo[4,5-c]quinolin-1-yl]-2-methylpropan-2-ol

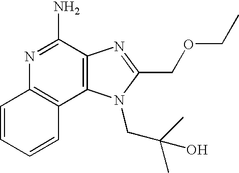

### Part A

Triethylamine (13.1 mL, 94.1 mmol) was added with stirring to a solution of 3-amino-4-chloroquinoline, see Surrey et al., Journal of the American Chemical Society, 73, pp. 2413-2416 (1951), (11.2 g, 62.7 mmol) in dichloromethane (125 mL). A solution of ethoxyacetyl chloride (9.2 g, 75 mmol) in dichloromethane (35 mL) was then added dropwise, and the reaction was stirred at room temperature overnight. An analysis by liquid chromatography/mass spectrometry (LC/MS) indicated the presence of starting material, and a solution of additional ethoxyacetyl chloride (2.3 g, 19 mmol) in dichloromethane (10 mL) was added dropwise. The reaction was stirred at room temperature overnight. Saturated aqueous sodium bicarbonate (100 mL) was added, and the resulting mixture was stirred at room temperature for 30 minutes. The organic layer was separated and washed sequentially with saturated aqueous sodium bicarbonate (50 mL) and water (2×50 mL), dried over potassium carbonate, filtered, and concentrated under reduced pressure to provide 17.0 g of N-(4-chloroquinolin-3-yl)-2-ethoxyacetamide as a dark oil that crystallized upon standing.

### Part B

A mixture of N-(4-chloroquinolin-3-yl)-2-ethoxyacetamide (4.5 g, 17 mmol), 1-amino-2-methylpropan-2-ol (2.3 g, 26 mmol), and p-toluenesulfonic acid monohydrate (150 mg, 0.79 mmol) was placed in a TEFLON-lined pressure vessel, heated at 125° C. for 15 hours, and allowed to cool to room temperature. Dichloromethane (150 mL) and saturated aqueous sodium bicarbonate (25 mL) were added, and the mixture was stirred for 15 minutes. The organic layer was separated and washed sequentially with saturated aqueous sodium bicarbonate (2×35 mL) and water (25 mL), dried over potassium carbonate, filtered, and concentrated under reduced pressure to provide 4.6 g of 1-[2-(ethoxymethyl)-1H-imidazo[4,5-c]quinolin-1-yl]-2-methylpropan-2-ol as a dark brown oil.

HRMS (ESI) calcd for C₁₇H₂₁N₃O₂ M+H⁺: 300.1712, found 300.1713.

### Part C

A solution of 1-[2-(ethoxymethyl)-1H-imidazo[4,5-c]quinolin-1-yl]-2-methylpropan-2-ol (0.98 g, 3.3 mmol) in dichloromethane (35 mL) was cooled to approximately 0° C., and 3-chloroperoxybenzoic acid (1.1 g of approximately 77% pure material, 5 mmol) was added. The reaction was stirred for ten minutes at 0° C., stirred for three hours at room temperature, diluted with dichloromethane (50 mL) and saturated aqueous sodium bicarbonate (35 mL), and stirred for 15 minutes. The aqueous layer was separated and extracted with dichloromethane (3×25 mL), and the combined organic fractions were dried over potassium carbonate, filtered, and concentrated under reduced pressure to provide 0.96 g of 1-[2-(ethoxymethyl)-5-oxido-1H-imidazo[4,5-c]quinolin-1-yl]-2-methylpropan-2-ol as a brown oil.

HRMS (ESI) calcd for C₁₇H₂₁N₃O₃ M+H⁺: 316.1661, found 316.1664.

### Part D

A solution of 1-[2-(ethoxymethyl)-5-oxido-1H-imidazo[4,5-c]quinolin-1-yl]-2-methylpropan-2-ol (0.96 g, 3.0 mmol) in dichloromethane (35 mL) was cooled to 0° C., and trichloroacetyl isocyanate (0.60 mL, 5.0 mmol) was added with stirring. The reaction was stirred for 15 minutes at 0° C. and then stirred overnight at room temperature. Methanol (10 mL) was added, and the mixture was stirred for 15 minutes and concentrated under reduced pressure. The residue was dissolved in methanol (10 mL), and sodium methoxide (0.25 mL of a 25% w/w solution in methanol) was added. The mixture was stirred at room temperature for three hours. An analysis by LC/MS indicated the reaction was incomplete, and additional sodium methoxide solution (1.0 mL) was added. The reaction was stirred overnight at room temperature and determined to be incomplete. Hydrochloric acid (5 mL of 10% w/w) was added, and the mixture was stirred for one hour. Saturated aqueous sodium bicarbonate (15 mL) and aqueous sodium hydroxide (ten drops of 50% w/w) were added, and the reaction was stirred overnight at room temperature. An analysis by LC/MS again showed that the reaction was incomplete. Potassium hydroxide (5 mL of a 0.5 N solution in methanol) was added, and the reaction mixture was heated at reflux for four hours, allowed to cool to room temperature, and concentrated under reduced pressure. The residue was dissolved in dichloromethane (75 mL), and the solution was washed with water (2×50 mL), dried over potassium carbonate, filtered, and concentrated under reduced pressure. The residue (0.64 g) was purified by column chromatography on silica gel (eluting with 5% methanol in dichloromethane containing 2 mL of aqueous ammonium hydroxide per liter of eluent). The resulting solid was recrystallized from methanol/water, recrystallized three times from methanol, and dried overnight under vacuum at 70° C. to provide 1-[4-amino-2-(ethoxymethyl)-1H-imidazo[4,5-c]quinolin-1-yl]-2-methylpropan-2-ol as tan needles, mp 192-194° C.

MS (APCI) m/z 315 (M+H⁺); HRMS (ESI) calcd for C₁₇H₂₂N₄O₂ M+H⁺: 315.1821, found 315.1819.

Anal. calcd for C₁₇H₂₂N₄O₂: C, 64.95; H, 7.05; N, 17.82. Found: C, 64.94; H, 6.94; N, 17.74.

### Example 2

### Preparation of N-[4-(4-Amino-2-ethyl-1H-imidazo[4,5-c]quinolin-1-yl)butyl]methanesulfonamide

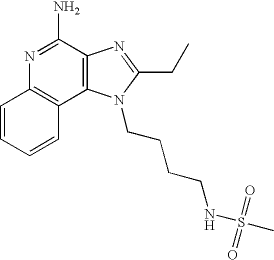

### Part A

A solution of 3-amino-4-chloroquinoline (5.0 g, 28 mmol) and triethylamine (5.8 mL, 42 mmol) in dichloromethane (100 mL) was cooled to approximately 0° C. A solution of propionyl chloride (2.8 g, 31 mmol) in dichloromethane (15 mL) was then added dropwise over a period of 15 minutes, and the reaction was allowed to warm to room temperature and stirred overnight. An analysis by high performance liquid chromatography (HPLC) indicated the presence of starting material, and additional triethylamine (1.95 mL, 14.0 mmol) and propionyl chloride (0.85 g, 9.2 mmol) in dichloromethane (5 mL) were added. The reaction was stirred at room temperature overnight; diluted with dichloromethane (100 mL); washed sequentially with water, saturated aqueous sodium carbonate, 10% w/w aqueous sodium hydroxide, saturated aqueous sodium carbonate, and brine; dried over magnesium sulfate and sodium sulfate, filtered, and concentrated under reduced pressure. The resulting brown solid (8.1 g) was recrystallized from toluene to provide 4.5 g of N-(4-chloroquinolin-3-yl)propanamide as beige platelets, mp 151-152° C.

### Part B

In a glass-lined pressure vessel, a solution of N-(4-chloroquinolin-3-yl)propanamide (3.3 g, 14 mmol), N-(4-aminobutyl)methanesulfonamide hydrochloride (see Example 199 in U.S. Patent Application Publication No. 2004/0147543, 3.14 g, 15.5 mmol), and triethylamine (3.9 mL, 28 mmol) in trifluoroethanol (35 mL) was heated at 150° C. for 16 hours and allowed to cool to room temperature. The volatiles were removed under reduced pressure, and the resulting amber paste was dissolved in dichloromethane. The solution was washed sequentially with dilute aqueous ammonium chloride, saturated aqueous sodium carbonate (2×), and brine; dried over magnesium sulfate and sodium sulfate; filtered; and concentrated under reduced pressure. The resulting amber syrup (4.8 g) was recrystallized from hot propyl acetate. The crystals were washed with propyl acetate to provide 3.8 g of N-[4-(2-ethyl-1H-imidazo[4,5-c]quinolin-1-yl)butyl]methanesulfonamide as light amber granules, mp 166-168° C.

### Part C

Solid 3-chloroperoxybenzoic acid (2.7 g of approximately 77% pure material, 13 mmol) was added in portions to a solution of N-[4-(2-ethyl-1H-imidazo[4,5-c]quinolin-1-yl)butyl]methanesulfonamide (3.8 g, 11 mmol) in dichloromethane (75 mL). The reaction was stirred for five hours at room temperature. An analysis by HPLC indicated the presence of starting material, and additional 3-chloroperoxybenzoic acid (0.5 g) was added. The reaction was stirred for one hour at room temperature, and ammonium hydroxide (50 mL) was added. The resulting mixture was vigorously stirred at room temperature for 15 minutes, and then p-toluenesulfonyl chloride (2.5 g, 0.013 mol) was added in one portion. The mixture was stirred vigorously at room temperature overnight. A fine solid was present and was collected by vacuum filtration (3.4 g) and recrystallized from ethanol (100 mL). The crystals were dried in a vacuum oven at 60° C. for four hours to provide 3.0 g of N-[4-(4-amino-2-ethyl-1H-imidazo[4,5-c]quinolin-1-yl)butyl]methanesulfonamide as straw-colored, feathery crystals, m.p. 198-200° C.

MS (APCI) m/z 362 (M+H)⁺;

Anal. calcd for C₁₇H₂₃N₅O₂S: C, 56.49; H, 6.41; N, 19.37. Found: C, 56.31; H, 6.49; N, 19.13.

### Example 3

### Preparation of N-{2-[4-Amino-2-(ethoxymethyl)-1H-imidazo[4,5-c]quinolin-1-yl]-1,1-dimethylethyl}methanesulfonamide monohydrate

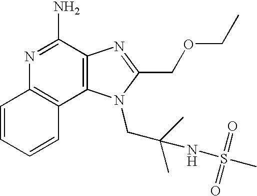

### Part A

A solution of 1,2-diamino-2-methylpropane (11.9 mL, 113 mmol), tert-butyl phenyl carbonate (42.1 mL, 227 mmol) and absolute ethanol (500 mL) was heated at reflux under a nitrogen atmosphere for 20.5 hours. The volatiles were removed under reduced pressure, and the residue was dissolved in water (560 mL). The solution was adjusted to approximately pH 3 with the addition of hydrochloric acid (140 mL of 1 N) and washed with dichloromethane (2×1 L). The aqueous solution was then adjusted to approximately pH 12 with the addition of aqueous sodium hydroxide (70 mL of 2 N) and extracted with dichloromethane (5×800 mL). The combined extracts were dried over sodium sulfate, filtered, concentrated under reduced pressure, and further dried under vacuum to provide 13.05 g of tert-butyl 2-amino-2-methylpropylcarbamate, which was combined with material from other runs.

### Part B

A solution of tert-butyl 2-amino-2-methylpropylcarbamate (20.8 g, 111 mmol) and triethylamine (23.2 mL, 167 mmol) in dichloromethane (125 mL) was cooled to −9° C. A solution of methanesulfonic anhydride (21.25 g, 122 mmol) in dichloromethane (106 mL) was added over a period of 50 minutes while maintaining the reaction temperature at or below −4° C. After the addition, the reaction was stirred for 30 minutes; diluted with dichloromethane (80 mL); washed sequentially with brine (30 mL), saturated aqueous ammonium chloride (30 mL), 10% w/w hydrochloric acid (20 mL), brine (10 mL), saturated aqueous sodium carbonate (20 mL), and brine (10 mL); dried over magnesium sulfate, filtered, concentrated under reduced pressure, and dried under vacuum to provide tert-butyl 2-methyl-2-[(methylsulfonyl)amino]propylcarbamate.

### Part C

Hydrogen chloride (153 mL of a 4 N solution in 1,4-dioxane) was cooled to 0° C. and stirred. The material from Part B was added in portions followed by an ethanol rinse. The reaction was allowed to warm to room temperature and stirred overnight. The solvent was removed under reduced pressure, and the residue was concentrated twice from ethanol and dried under vacuum to provide N-(2-amino-1,1-dimethylethyl)methanesulfonamide hydrochloride.

### Part D

In a glass-lined pressure vessel, a solution of N-(4-chloroquinolin-3-yl)-2-ethoxyacetamide (2.0 g, 7.6 mmol), N-(2-amino-1,1-dimethylethyl)methanesulfonamide hydrochloride (2.16 g, 10.7 mmol), and triethylamine (2.6 mL, 19 mmol) in trifluoroethanol (30 mL) was heated at −150° C. for 16 hours. An analysis by HPLC indicated the presence of starting material, and additional N-(2-amino-1,1-dimethylethyl)methanesulfonamide hydrochloride (6.5 g) and triethylamine (0.3 mL) were added. The heating was continued for an additional 16 hours. The volatiles were removed under reduced pressure, and the resulting amber paste was dissolved in dichloromethane. The solution was washed sequentially with dilute aqueous ammonium chloride, saturated aqueous sodium carbonate (2×), and brine; dried over magnesium sulfate and sodium sulfate; filtered; and concentrated under reduced pressure. The resulting amber syrup (4 g) was purified by column chromatography on silica gel to provide 1.6 g of N-{2-(ethoxymethyl)-1H-imidazo[4,5-c]quinolin-1-yl]-1,1-dimethylethyl}methanesulfonamide as an amber syrup.

### Part E

Solid 3-chloroperoxybenzoic acid (1.1 g of approximately 77% pure material, 5 mmol) was added in portions to absolution of N-{2-(ethoxymethyl)-1H-imidazo[4,5-c]quinolin-1-yl]-1,1-dimethylethyl}methanesulfonamide (1.6 g, 4.2 mmol) in dichloromethane (30 mL). The reaction was stirred for three hours at room temperature. An analysis by HPLC indicated the presence of a small amount of starting material, and additional 3-chloroperoxybenzoic acid (0.2 g) was added. The reaction was stirred for one hour at room temperature, and ammonium hydroxide (30 mL) was added. The resulting mixture was vigorously stirred at room temperature for 15 minutes, and then p-toluenesulfonyl chloride (0.97 g, 0.0051 mol) was added in one portion. The reaction mixture was vigorously stirred at room temperature for two hours, diluted with dichloromethane, washed sequentially with saturated aqueous sodium carbonate (2×) and brine (1×), dried over sodium sulfate and magnesium sulfate, filtered, and concentrated under reduced pressure to give an amber syrup. The syrup was purified by column chromatography on silica gel (eluting with dichloromethane) followed by recrystallization from propyl acetate (10 mL/g) to give 0.8 g of a tan solid. The solid was recrystallized from ethanol (4 mL) and water (two drops), and the crystals were dried in a vacuum oven at 60° C. for four hours to provide 0.55 g of N-{2-[4-amino-2-(ethoxymethyl)-1H-imidazo[4,5-c]quinolin-1-yl]-1,1-dimethylethyl}methanesulfonamide monohydrate as pale yellow crystals, m.p. 161-163° C.

MS (ESI) m/z 392 (M+H)⁺;

Anal. calcd for C₁₈H₂₅N₅O₃S.1.0H₂O: C, 52.79; H, 6.65; N, 17.10. Found: C, 52.69; H, 6.56; N, 16.87.

### Example 4

### Preparation of N-[4-(2-Ethyl-1H-imidazo[4,5-c]quinolin-1-yl)butyl]methanesulfonamide

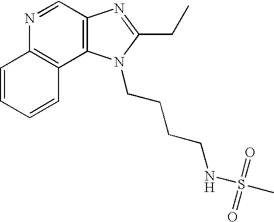

A mixture of N-(4-chloroquinolin-3-yl)propanamide (0.1 g, 0.4 mmol) and N-(4-aminobutyl)methanesulfonamide hydrochloride (0.095 g, 0.5 mmol) was sealed in a vial, heated at 125° C. for 16 hours, and allowed to cool to room temperature. Water was added, and the solution was adjusted to approximately pH 10 with the addition of sodium carbonate. The aqueous solution was extracted twice with dichloromethane (50 mL), and the combined extracts were washed with brine, dried over magnesium sulfate and sodium sulfate, filtered, and concentrated under reduced pressure. Analysis by HPLC and mass spectrometry gave evidence that N-[4-(2-ethyl-1H-imidazo[4,5-c]quinolin-1-yl)butyl]methanesulfonamide was the major product. MS (APCI) W/z 347 (M+H)⁺.

### Examples 5 and 6

### Preparation of N-[4-(2-Ethyl-1H-imidazo[4,5-c]quinolin-1-yl)butyl]methanesulfonamide

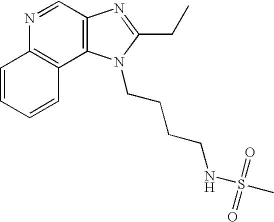

A solution of N-(4-chloroquinolin-3-yl)propanamide (0.05 g, 0.2 mmol), N-(4-aminobutyl)methanesulfonamide hydrochloride (0.047 g, 0.2 mmol), and triethylamine (0.059 mL, 0.40 mmol) in the solvent indicated below was sealed in a vial and heated at 128° C. for 48 hours. An analysis by HPLC indicated the ratio of N-[4-(2-ethyl-1H-imidazo[4,5-c]quinolin-1-yl)butyl]methanesulfonamide to N-(4-chloroquinolin-3-yl)propanamide shown below.

| Example | Solvent | Product:Starting Material |
|---|---|---|
| 5 | tert-butanol | 93:7 |
| 6 | isopropanol | 92:8 |

### Example 7

### Preparation of 2-Methyl-1-(2-methylpropyl)-1H-imidazo[4,5-c][1,5]naphthyridin-4-amine

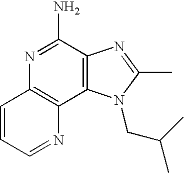

### Part A

A Parr vessel was flushed with nitrogen and charged with 5% platinum on carbon (1.1 g) and water (5 mL). A solution of 3-nitro[1,5]naphthyridin-4-ol (22.0 g, 0.115 mol) in triethylamine (23.3 g, 0.230 mol) and water (1.1 L) was added, and the mixture was placed under hydrogen pressure (35 psi, 2.4×10⁵ Pa) for three hours and then filtered through a layer of CELITE filter agent. The filtrate was concentrated to a volume of 400 mL by flash evaporation at 75° C. and allowed to cool. A solid formed and was collected by vacuum filtration to give 3-amino[1,5]naphthyridin-4-ol (16.4 g) as a fine mustard-colored powder, mp 315-320° C.

MS (ESI) m/z 162 (M+H)⁺.

### Part B

Phosphorus(III) oxychloride (47.0 g, 0.304 mol) was added dropwise over a period of 30 minutes to a mixture of 3-amino[1,5]naphthyridin-4-ol (20.0 g, 0.124 mol) in N,N-dimethylformamide (DMF) (200 mL); the reaction temperature was maintained between 10° C. and 20° C. during the addition. When the addition was complete, the reaction was stirred at room temperature for three hours, heated to 90° C. for 15 minutes, and allowed to cool to room temperature. Water (150 mL) and ice were added while maintaining the temperature below 55° C. The mixture was stirred at room temperature for 30 minutes, heated at 100° C. for three hours, and allowed to cool to room temperature overnight. The resulting black solution was made basic by slowly adding solid sodium carbonate and saturated aqueous sodium carbonate. The basic solution was extracted three times with dichloromethane. The combined extracts were washed with brine, dried over sodium sulfate and magnesium sulfate, and concentrated under reduced pressure to give a sticky amber paste. The paste was triturated with warm diethyl ether for 30 minutes, and the resulting solid was collected by vacuum filtration to give 4-chloro[1,5]naphthyridin-3-amine (11 g) as brown granules, mp 188-190° C.

MS (ESI) m/z 180 (M+H)⁺.

### Part C

A solution of 4-chloro[1,5]naphthyridin-3-amine (4.0 g, 0.022 mol) and triethylamine (4.6 mL, 0.033 mol) in 1,2-dichloroethane (80 mL) was cooled to 3° C. A solution of acetyl chloride (4.4 g, 0.056 mol) in 1,2-dichloroethane (5 mL) was added dropwise over a period of five minutes. The reaction was then heated at reflux for five hours; allowed to cool to room temperature; diluted with dichloromethane (100 mL); washed sequentially with saturated aqueous sodium carbonate (2×), water (1×), and brine (1×); dried over sodium sulfate and magnesium sulfate; filtered; and concentrated under reduced pressure to give 4.7 g of dark brown solid. The solid was recrystallized from acetonitrile (20 mL) and collected in two crops to give N-(4-chloro[1,5]naphthyridin-3-yl)acetamide (3.0 g) as brown solids.

MS (ESI) m/z 222 (M+H)⁺.

### Part D

A mixture of N-(4-chloro[1,5]naphthyridin-3-yl)acetamide (1.6 g, 0.073 mol) and isobutylamine (7.3 mL, 0.0722 mol) was heated in a sealed vial at 120° C. overnight, allowed to cool to room temperature, and added to a solution of water (100 mL) and saturated aqueous sodium carbonate (20 mL). A precipitate formed. The mixture was stirred at room temperature for 1.5 hours, and the precipitate was collected by vacuum filtration and dried on the filter funnel to give 2-methyl-1-(2-methylpropyl)-1H-imidazo[4,5-c][1,5]naphthyridine (1.45 g) as gray powder, mp 110-111° C.

MS (APCI) m/z 241 (M+H)⁺.

### Part E

Solid 3-chloroperoxybenzoic acid (1.8 g of approximately 77% pure material, 0.0078 mol) was added in portions to a solution of 2-methyl-1-(2-methylpropyl)-1H-imidazo[4,5-c][1,5]naphthyridine (1.45 g, 0.0060 mol) in dichloromethane (30 mL). The reaction was stirred at room temperature for three hours and washed with saturated aqueous sodium carbonate (100 mL). The aqueous fraction was extracted three times with dichloromethane. The combined organic fractions were washed with brine, dried over sodium sulfate and magnesium sulfate, filtered, and concentrated under reduced pressure to give 2-methyl-1-(2-methylpropyl)-5-oxido-1H-imidazo[4,5-c][1,5]naphthyridine (1.3 g) as yellow solid.

MS (APCI) m/z 257 (M+H)⁺.

### Part F

p-Toluenesulfonyl chloride (1.2 g, 0.0061 mol) was added to a vigorously stirred mixture of 2-methyl-1-(2-methylpropyl)-5-oxido-1H-imidazo[4,5-c][1,5]naphthyridine (1.3 g, 0.0051 mol), dichloromethane (25 mL), and ammonium hydroxide (17 mL). The reaction was vigorously stirred at room temperature for two hours, and then the stirring was stopped and the layers allowed to separate for two hours. A precipitate formed and was collected by vacuum filtration to give 0.9 g of ivory needles. The needles were recrystallized from isopropanol (12 mL), and the collected crystals were dried in a vacuum oven at 60° C. for five hours to give 2-methyl-1-(2-methylpropyl)-1H-imidazo[4,5-c][1,5]naphthyridin-4-amine (0.7 g) as colorless needles, mp 227-229° C.

MS (ESI) m/z 256 (M+H)⁺;

Anal. calcd for C₁₄H₁₇N₅: C, 65.86; H, 6.71; N, 27.43. Found: C, 65.53; H, 6.68; N, 27.35.

### Example 8

### Preparation of 1-(2-Methylpropyl)-1H-imidazo[4,5-c]quinoline

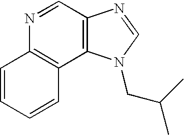

### Part A

Formic acid (0.36 mL) was slowly added with stirring to acetic anhydride (0.8 mL), and the reaction was stirred at room temperature for 2.75 hours and then added to a solution of 3-amino-4-chloroquinoline (0.50 g, 2.8 mmol) in tetrahydrofuran (5 mL). The resulting mixture was stirred for one hour at room temperature. A solid was present and was collected by filtration and washed with diethyl ether to provide 0.48 g of 4-chloroquinolin-3-ylformamide as a beige solid, mp 175-177° C.

### Part B

A mixture of 4-chloroquinolin-3-ylformamide (0.050 g, 0.24 mol) and isobutylamine (0.25 mL, 2.4 mmol) was heated in a sealed vial at 110° C. overnight and allowed to cool to room temperature. Dichloromethane (1 mL) and aqueous ammonium chloride (1 mL of 10% w/w) were added. The organic layer was separated and washed with 10% w/w aqueous ammonium chloride, dried over sodium sulfate, concentrated under reduced pressure, and dried overnight in a vacuum oven at 35° C. to provide 41 mg of a brown crystalline solid that was analyzed by LC/MS and found to be a mixture of 1-(2-methylpropyl)-1H-imidazo[4,5-c]quinoline and 3-amino-4-chloroquinoline.

### Example 9

### Preparation of 1-(2-Methylpropyl)-1H-imidazo[4,5-c]quinoline

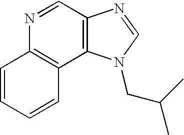

### Part A

Phosphorous oxychloride (90 mL, 0.97 mol) was added dropwise to a stirred slurry of 3-aminoquinolin-4-ol hydrochloride (150 g, 0.76 mol) in 500 mL of DMF. The temperature of the mixture increased to about 100° C. over the course of the addition. The reaction mixture was allowed to cool to room temperature, then about two-thirds of the DMF was removed under reduced pressure. The mixture was then filtered and the solid dried to provide N′-(4-chloroquinolin-3-yl)-N,N-dimethylimidoformamide (118 g),

MS (ESI) m/z 234 (M+H)⁺.

### Part B

A glass vial was charged with N′-(4-chloroquinolin-3-yl)-N,N-dimethylimidoformamide (0.10 g, 0.40 mmol), isobutylamine (0.50 mL, 5.4 mmol), and pyridinium p-toluenesulfonate (5 mg, 0.02 mmol). The vial was placed inside a steel pressure reactor and the vessel heated in an oven at 150° C. for 15 hours. After cooling to room temperature, examination of the reaction mixture by liquid chromatography/mass spectrometry indicated that the reaction was incomplete, so the vessel was placed back inside the oven and heated at 175° C. for 15 hours. After cooling to room temperature, examination of the reaction mixture by liquid chromatography/mass spectrometry indicated that the major product of the reaction was 1-(2-methylpropyl)-1H-imidazo[4,5-c]quinoline, MS (ESI) m/z 226 (M+H)⁺.

### Example 10

### Preparation of 1-(2-Methylpropyl)-1H-imidazo[4,5-c]quinoline

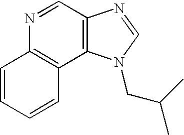

A flask was charged with 3-aminoquinolin-4-ol hydrochloride (1.0 g, 5.1 mmol), 20 mL of acetonitrile and 1.5 mL of DMF. Phosphorous oxychloride (0.7 mL, 7.5 mmol) was added to the stirred slurry. The mixture was stirred at ambient temperature for 21 hours. The mixture was filtered to remove solids, rinsing with acetonitrile. To the filtrates was added isobutylamine (2.5 mL, 25.5 mmol). An exotherm was observed upon addition and the solution became a slurry. The mixture was placed in a glass pressure vessel and heated in an oven at 120° C. for 3 hours. After cooling to room temperature, examination of the reaction mixture by liquid chromatography/mass spectrometry indicated that the reaction was mainly complete. The slurry was taken up in 50 mL of water and 50 mL of dichloromethane. The layers were separated. The aqueous was extracted with 25 mL of dichloromethane, which was combined with the separated dichloromethane layer. Examination of the combined dichloromethane layers by liquid chromatography/mass spectrometry indicated that the major product was 1-(2-methylpropyl)-1H-imidazo[4,5-c]quinoline, MS (ESI) m/z 226 (M+H)⁺. The layers were dried with magnesium sulfate, filtered and concentrated to provide 0.17 g (15%) of a sticky amber residue. ¹H NMR (300 MHz, DMSO-d₆) δ 9.24 (s, 1H), 8.43 (s, 1H), 8.35 (m, 1H), 8.18 (m, 1H), 7.74 (m, 2H), 4.53 (d, J=7.5 Hz, 2H), 2.22 (m, 1H), 0.93 (d, J=6.6 Hz, 6H); HRMS (ESI) calcd for C₁₄H₁₅N₃ [M+H]⁺: 226.1344, found 226.1352.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. A method for preparing a 1H-imidazo[4,5-c]pyridine compound or analog thereof or a pharmaceutically acceptable salt thereof comprising:
providing a compound of the Formula IV: 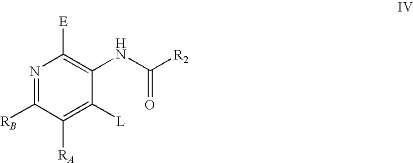
and reacting the compound of Formula IV with an amine of the formula R₁NH₂ to provide a 1H-imidazo[4,5-c]pyridine or analog thereof of the Formula I: 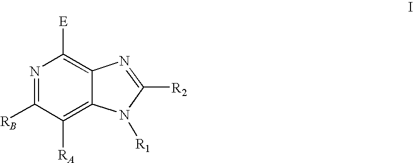
or a pharmaceutically acceptable salt thereof,
wherein:
E is selected from the group consisting of hydrogen, fluoro, chloro, bromo, iodo, hydroxy, phenoxy, —O—S(O)₂—R′, and —N(Bn)₂, wherein R′ is selected from the group consisting of alkyl, haloalkyl, and aryl optionally substituted by alkyl, halo, or nitro, and Bn is selected from the group consisting of benzyl, p-methoxybenzyl, p-methylbenzyl, and 2-furanylmethyl; or
E is joined with the adjacent pyridine nitrogen atom of Formulas I and IV to form the fused tetrazolo ring in Formulas I-1 and IV-1: 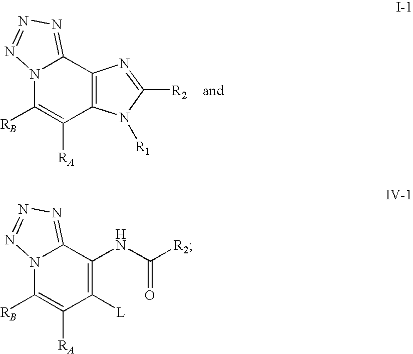
L is selected from the group consisting of fluoro, chloro, bromo, iodo, phenoxy, and —O—S(O)₂—R′, wherein R′ is selected from the group consisting of alkyl, haloalkyl, and aryl optionally substituted by alkyl, halo, or nitro;
R_{A} and R_{B} are independently selected from the group consisting of:
hydrogen,
halogen,
alkyl,
alkenyl,
alkoxy,
alkylthio, and
—N(R₉)₂;
or R_{A} and R_{B} taken together form a fused benzene ring or a fused pyridine ring wherein the benzene ring or pyridine ring is unsubstituted or substituted by one R group, or substituted by one R₃ group, or substituted by one R group and one R₃ group;
or R_{A} and R_{B} taken together form a fused 5 to 7 membered saturated ring optionally containing one nitrogen atom, wherein the fused ring is unsubstituted or substituted by one or more R groups;
R is selected from the group consisting of:
halogen,
hydroxy,
alkyl,
alkenyl,
haloalkyl,
alkoxy,
alkylthio, and
—N(R₉)₂;
R₁ is selected from the group consisting of:
-R₄,
-X-R₄,
-X-Y-R₄,
-X-Y-X-Y-R₄,
-X-R₅,
—N(R₁′)-Q-R₄,
—N(R₁′)—X₁—Y—R₄, and
—N(R₁′)—X₁—R_{5b};
R₂ is selected from the group consisting of:
-R₄,
-X-R₄,
-X-Y-R₄, and
-X-R₅;
R₃ is selected from the group consisting of:
-Z-R₄
-Z-X-R₄,
-Z-X-Y-R₄,
-Z-X-Y-X-Y-R₄, and
-Z-X-R₅;
X is selected from the group consisting of alkylene, alkenylene, alkynylene, alkylene, heteroarylene, and heterocyclylene wherein the alkylene, alkenylene, and alkynylene groups can be optionally interrupted or terminated by arylene, heteroarylene or heterocyclylene and optionally interrupted by one or more —O— groups;
X₁ is C₂₋₂₀ alkylene;
Y is selected from the group consisting of:
—O—,
—S(O)₀₋₂—,
—S(O)₂—N(R₈)—,
—C(R₆)—,
—O—C(R₆)—,
—O—C(O)—O—,
—N(R₈)-Q-,
—O—C(R₆)—N(R)—,
—C(R₆)—N(OR₉)—,
—O—N(R₈)-Q-,
—O—N═C(R₄)—,
—C(═N—O—R₈)—,
—CH(—N(—O—R₈)-Q-R₄)—, 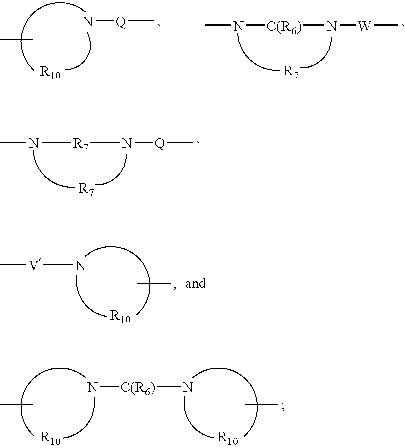
Y₁ is selected from the group consisting of —O—, —S(O)₀₋₂—, —S(O)₂—N(R₈)—, —N(R₈)-Q-, —C(R₆)—N(R₈)—, —O—C(R₆)—N(R₈)—, and 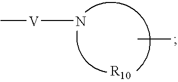
Z is a bond or —O—;
R₁′ is selected from the group consisting of hydrogen, C₁₋₂₀ alkyl, hydroxy-C₂₋₂₀ alkylenyl, and alkoxy-C₂₋₂₀ alkylenyl;
R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;
R₅ is selected from the group consisting of: 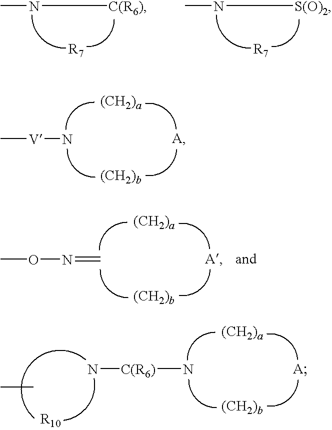
R_{5b} is selected from the group consisting of: 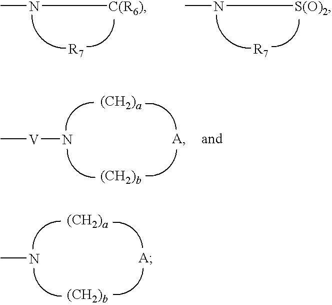
R₆ is selected from the group consisting of ═O and —S;
R₇ is C₂₋₇ alkylene;
R₈ is selected from the group consisting of hydrogen, alkyl, alkoxyalkylenyl, hydroxyalkylenyl, arylalkylenyl, and heteroarylalkylenyl;
R₉ is selected from the group consisting of hydrogen and alkyl;
R₁₀ is C₁₋₈ alkylene;
A is selected from the group consisting of —O—, —C(O)—, —S(O)₀₋₂-, and N(R₄)—;
A′ is selected from the group consisting of —O—, —S(O)₀₋₂—, —N(-Q-R₄)—, and —CH₂—;
Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, —C(R₆)—S—, and —C(R₆)—N(R₆)—;
V is selected from the group consisting of —C(R₆)—, —O—C(R₆)—, —N(R₈)—C(R₆)—, and —S(O)₂—;
V′ is selected from the group consisting of —O—C(R₆)—, —N(R₈)—C(R₆)—, and —S(O)₂—;
W is selected from the group consisting of a bond, —C(O)—, and —S(O)₂—; and
a and b are independently integers from 1 to 6 with the proviso that a+b is ≦7.

2.-7. (canceled)

8. A method for preparing a 1H-imidazo[4,5-c]pyridine compound or analog thereof or a pharmaceutically acceptable salt thereof comprising:
providing a compound of the Formula VIII: 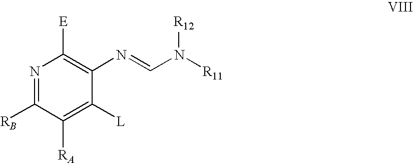
and reacting the compound of Formula VIII with an amine of the formula R₁NH₂ to provide a 1H-imidazo[4,5-c]pyridine or analog thereof of the Formula I: 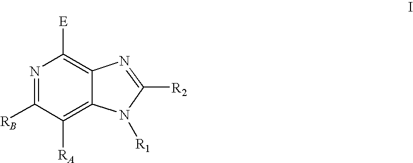
or a pharmaceutically acceptable salt thereof
wherein:
E is selected from the group consisting of hydrogen, fluoro, chloro, bromo, iodo, hydroxy, phenoxy, —O—S(O)₂—R′, and —N(Bn)₂, wherein R′ is selected from the group consisting of alkyl, haloalkyl, and aryl optionally substituted by alkyl, halo, or nitro, and Bn is selected from the group consisting of benzyl, p-methoxybenzyl, p-methylbenzyl, and 2-furanylmethyl; or
E is joined with the adjacent pyridine nitrogen atom of Formulas I and VIII to form the fused tetrazolo ring in Formulas I-1 and IX: 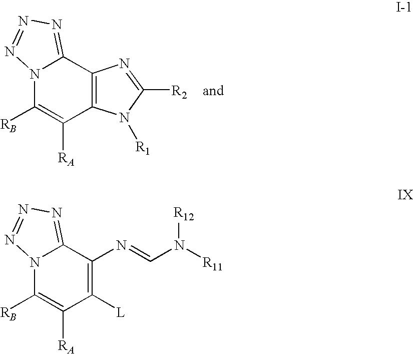
L is selected from the group consisting of fluoro, chloro, bromo, iodo, phenoxy, and —O—S(O)₂—R′, wherein R′ is selected from the group consisting of alkyl, haloalkyl, and aryl optionally substituted by alkyl, halo, or nitro;
R_{A} and R_{B} are independently selected from the group consisting of:
hydrogen,
halogen,
alkyl,
alkenyl,
alkoxy,
alkylthio, and
—N(R₉)₂;
or R_{A} and R_{B} taken together form a fused benzene ring or a fused pyridine ring wherein the benzene ring or pyridine ring is unsubstituted or substituted by one R group, or substituted by one R₃ group, or substituted by one R group and one R₃ group;
or R_{A} and R_{B} taken together form a fused 5 to 7 membered saturated ring optionally containing one nitrogen atom, wherein the fused ring is unsubstituted or substituted by one or more R groups;
R is selected from the group consisting of:
halogen,
hydroxy,
alkyl,
alkenyl,
haloalkyl,
alkoxy,
alkylthio, and
—N(R₉)₂;
R₁ is selected from the group consisting of:
-R₄,
-X-R₄,
-X-Y-R₄,
-X-Y-X-Y-R₄,
-X-R₅,
—N(R₁′)-Q-R₄,
—N(R₁′)—X₁—Y₁—R₄, and
—N(R₁′)—X₁—R_{5b};
R₂ is hydrogen;
R₃ is selected from the group consisting of:
-Z-R₄
-Z-X-R₄,
-Z-X-Y-R₄,
-Z-X-Y-X-Y-R₄, and
-Z-X-R₅;
X is selected from the group consisting of alkylene, alkenylene, alkynylene, arylene, heteroarylene, and heterocyclylene wherein the alkylene, alkenylene, and alkynylene groups can be optionally interrupted or terminated by arylene, heteroarylene or heterocyclylene and optionally interrupted by one or more —O— groups;
X₁ is C₂₋₂₀ alkylene;
Y is selected from the group consisting of:
—O—,
—S(O)₀₋₂—,
—S(O)₂—N(R₈)—,
—C(R₆)—,
—O—C(R₆)—,
—O—C(O)—O—,
—N(R₈)-Q-,
—O—C(R₆)—N(R₈)—,
—C(R₆)—N(OR₉)—,
—O—N(R₈)-Q-,
—O—N═C(R₄)—,
—C(—N—O—R₈)—,
—CH(—N(—O—R₈)-Q-R₄)—, 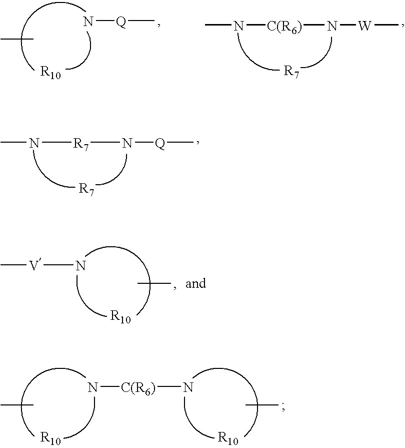
Y₁ is selected from the group consisting of —O—, —S(O)₀₋₂—, —S(O)₂—N(R₈)—, —N(R₈)-Q-, —C(R₆)—N(R₈)—, —O—C(R₈)—N(R₈)—, and 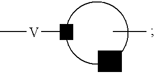
Z is a bond or —O—;
R₁′ is selected from the group consisting of hydrogen, C₁₋₂₀ alkyl, hydroxy-C₂₋₂₀ alkylenyl, and alkoxy-C₂₋₂₀ alkylenyl;
R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;
R₅ is selected from the group consisting of: 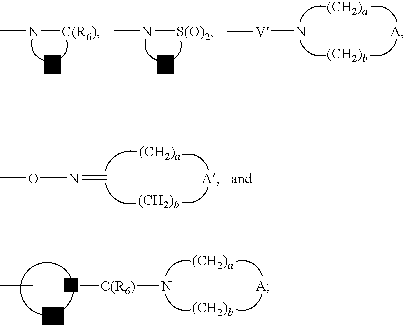
R_{5b} is selected from the group consisting of: 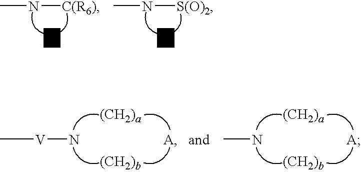
R₆ is selected from the group consisting of ═O and ═S;
R₇ is C₂₋₇ alkylene;
R₈ is selected from the group consisting of hydrogen, alkyl, alkoxyalkylenyl, hydroxyalkylenyl, arylalkylenyl, and heteroarylalkylenyl;
R₉ is selected from the group consisting of hydrogen and alkyl;
R₁₀ is C₃₋₈ alkylene;
R₁₁ and R₁₂ are independently C₁₋₄ alkyl or R₁₁ and R₁₂ together with the nitrogen atom to which they are attached form a 5- or 6-membered ring optionally containing —O—, —N(C₁₋₄ alkyl)-, or —S—;
A is selected from the group consisting of —O—, —C(O)—, —S(O)₀₋₂—, and —N(R₄)—;
A′ is selected from the group consisting of —O—, —S(O)₀₋₂—, —N(-Q-R₄)—, and —CH₂—;
Q is selected from the group consisting of a bond, —C(R₆)—, —C(R₆)—C(R₆)—, —S(O)₂; —C(R₆)—N(R₈)—W—, —S(O)₂—N(R₈)—, —C(R₆)—O—, —C(R₆)—S—, and —C(R₆)—N(OR₉)—;
V is selected from the group consisting of —C(R₆)—, —O—C(R₆)—, —N(R₈)—C(R₆)—, and —S(O)₂—;
V′ is selected from the group consisting of —O—C(R₆)—, —N(R₈)—C(R)—, and —S(O)₂—;
W is selected from the group consisting of a bond, —C(O)—, and —S(O)₂—; and
a and b are independently integers from 1 to 6 with the proviso that a+b is ≦7.

9.-12. (canceled)

13. A method for preparing a 1H-imidazo[4,5-c]pyridine compound or analog thereof or a pharmaceutically acceptable salt thereof comprising:
providing a compound of the Formula XI: 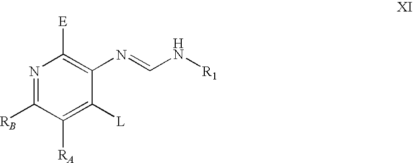
and forming a 1H-imidazo[4,5-c]pyridine or analog thereof of the Formula I: 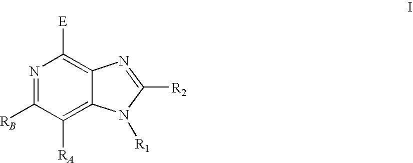
or a pharmaceutically acceptable salt thereof;
wherein:
E is selected from the group consisting of hydrogen, fluoro, chloro, bromo, iodo, hydroxy, phenoxy, —O—S(O)₂—R′, and —N(Bn)₂, wherein R′ is selected from the group consisting of alkyl, haloalkyl, and aryl optionally substituted by alkyl, halo, or nitro, and Bn is selected from the group consisting of benzyl, p-methoxybenzyl, p-methylbenzyl, and 2-furanylmethyl; or
E is joined with the adjacent pyridine nitrogen atom of Formulas I and XI to form the fused tetrazolo ring in Formulas I-1 and XIII: 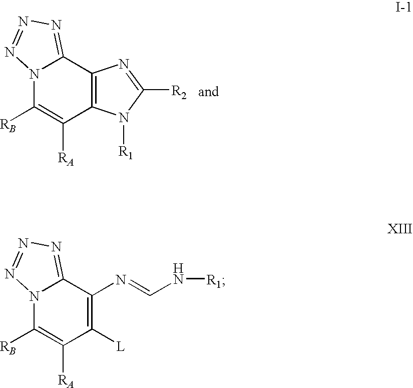
L is selected from the group consisting of fluoro, chloro, bromo, iodo, phenoxy, and —O—S(O)₂—R′, wherein R′ is selected from the group consisting of alkyl, haloalkyl, and aryl optionally substituted by alkyl, halo, or nitro;
R_{A} and R_{B} are independently selected from the group consisting of:
hydrogen,
halogen,
alkyl,
alkenyl,
alkoxy,
alkylthio, and
—N(R₉)₂;
or R_{A} and R_{B} taken together form a fused benzene ring or a fused pyridine ring wherein the benzene ring or pyridine ring is unsubstituted or substituted by one R group, or substituted by one R₃ group, or substituted by one R group and one R₃ group;
or R_{A} and R_{B} taken together form a fused 5 to 7 membered saturated ring optionally containing one nitrogen atom, wherein the fused ring is unsubstituted or substituted by one or more R groups;
R is selected from the group consisting of:
halogen,
hydroxy,
alkyl,
alkenyl,
haloalkyl,
alkoxy,
alkylthio, and
—N(R₉)₂;
R₁ is selected from the group consisting of:
-R₄,
-X-R₄,
-X-Y-R₄,
-X-Y-X-Y-R₄,
-X-R₅,
—N(R₁′)-Q-R₄,
—N(R₁′)—X₁—Y₁—R₄, and
—N(R₁′)—X₁—R_{5b};
R₂ is hydrogen;
R₃ is selected from the group consisting of:
-Z-R₄,
-Z-X-R₄,
-Z-X-Y-R₄,
-Z-X-Y-X-Y-R₄, and
-Z-X-R₅;
X is selected from the group consisting of alkylene, alkenylene, alkynylene, arylene, heteroarylene, and heterocyclylene wherein the alkylene, alkenylene, and alkynylene groups can be optionally interrupted or terminated by arylene, heteroarylene or heterocyclylene and optionally interrupted by one or more —O— groups;
X₁ is C₂₋₂₀ alkylene;
Y is selected from the group consisting of:
—O—,
—S(O)₀₋₂—,
—S(O)₂—N(R₈)—,
—C(R₆)—,
—O—C(R₆)—,
—O—C(O)—O—,
—N(R₈)-Q-,
—O—C(R₆)—N(R₈)—,
—C(R₆)—N(OR₉)—,
—O—N(R₈)-Q-,
—O—N═C(R₄)—,
—C(═N—O—R₈)—,
—CH(—N(—O—R₈)-Q-R₄)—, 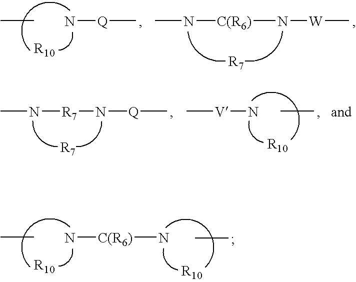
Y₁ is selected from the group consisting of —O—, —S(O)₀₋₂—, —S(O)₂—N(R₈)—, —N(R₈)-Q-, —C(R₆)—N(R₈)—, —O—C(R₆)—N(R₈)—, and 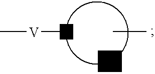
Z is a bond or —O—;
R₁′ is selected from the group consisting of hydrogen, C₁₋₂₀ alkyl, hydroxy-C₂₋₂₀ alkylenyl, and alkoxy-C₂₋₂₀ alkylenyl;
R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;
R₅ is selected from the group consisting of: 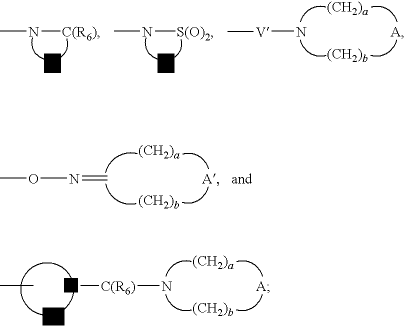
R_{5b} is selected from the group consisting of: 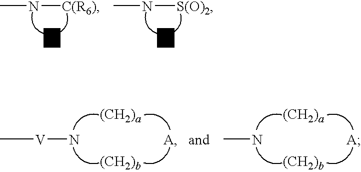
R₆ is selected from the group consisting of ═O and ═S;
R₇ is C₂₋₇ alkylene,
R₈ is selected from the group consisting of hydrogen, alkyl, alkoxyalkylenyl, hydroxyalkylenyl, arylalkylenyl, and heteroarylalkylenyl;
R₉ is selected from the group consisting of hydrogen and alkyl;
R₁₀ is C₃₋₈ alkylene;
A is selected from the group consisting of —O—, —C(O)—, —S(O)₀₋₂—, and —N(R₄)—;
A′ is selected from the group consisting of —O—, —S(O)₀₋₂—, —N(-Q-R₄)—, and —CH₂—;
Q is selected from the group consisting of a bond, —C(R₆)—, —C(R₆)—C(R₆)—, —S(O)₂—, —C(R₆)—N(R₈)—W—, —S(O)_{z}—N(R₈)—, —C(R₆)—O—, —C(R₆)—S—, and —C(R₆)—N(OR₉)—;
V is selected from the group consisting of —C(R₆)—, —O—C(R₆)—, —N(R₈)—C(R₆)—, and —S(O)₂;
V′ is selected from the group consisting of —O—C(R₆)—, —N(R₈)—C(R₆)—, and —S(O)₂—;
W is selected from the group consisting of a bond, —C(O)—, and —S(O)₂—; and
a and b are independently integers from 1 to 6 with the proviso that a+b is ≦7.

14.-16. (canceled)

17. The method of claim 1 further comprising the step of converting E to an amino group in the compound of Formula I to provide a compound of the Formula X: 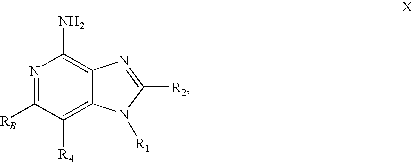
or a pharmaceutically acceptable salt thereof.

18.-26. (canceled)

27. The method of claim 1 wherein R₂ is -R₄.

28. The method of claim 27 wherein R₂ is selected from the group consisting of hydrogen, methyl, ethyl, propyl, butyl, 2-methoxyethyl, 2-hydroxyethyl, ethoxymethyl, and hydroxymethyl.

29.-31. (canceled)

32. The method of claim 1 wherein R₁ is -X-Y-R₄.

33. (canceled)

34. The method of claim 32 wherein -X-Y-R₄ is selected from the group consisting of 2-propylsulfonyl)ethyl, 2-methyl-2-[(methylsulfonyl)amino]propyl, 4-methylsulfonylaminobutyl, and 2-(acetylamino)-2-methylpropyl.

35.-38. (canceled)

39. The method of claim 1 wherein R_{A} and R_{B} taken together form a fused benzene ring wherein the benzene ring is unsubstituted or substituted by one R group, or substituted by one R₃ group, or substituted by one R group and one R₃ group.

40.-45. (canceled)

46. The method of claim 1 wherein R_{A} is R_{A1}, R_{B} is R_{B1}, R₁ is R₁ₐ, and R₂ is R₂ₐ, wherein:
R_{A1} and R_{B1} are independently selected from the group consisting of:
hydrogen,
halogen,
alkyl,
alkenyl,
alkoxy,
alkylthio, and
—N(R₉)₂;
or R_{A1} and R_{B}, taken together form a fused benzene ring or a fused pyridine ring wherein the benzene ring or pyridine ring is unsubstituted or substituted by one Rₐ group, or substituted by one R₃ₐ group, or substituted by one Rₐ group and one R₃ₐ group;
or R_{A1} and R_{B1} taken together form a fused 5 to 7 membered saturated ring optionally containing one nitrogen atom, wherein the fused ring is unsubstituted or substituted by one or more Rₐ groups;
Rₐ is selected from the group consisting of:
halogen,
hydroxy,
alkyl,
alkenyl,
trifluoromethyl,
alkoxy,
alkylthio, and
—N(R₉)₂;
R₁ₐ is selected from the group consisting of:
-R₄ₐ,
-X-R₄ₐ,
-X-Yₐ-R₄ₐ,
-X-R₅ₐ,
—N(R₁′)-Q-R₄ₐ,
—N(R₁′)—X₁—Y₁—R₄ₐ, and
—N(R₁′)—X₁—R_{5b};
R₂ₐ is selected from the group consisting of:
-R₄ₐ,
-X-R₄ₐ,
-X-Yₐ-R₄ₐ, and
-X-R₅ₐ;
R₃ₐ is selected from the group consisting of:
-Z-R₄ₐ,
-Z-X-R₄ₐ,
-Z-X-Yₐ-R₄ₐ,
-Z-X-Yₐ-X-Yₐ-R₄ₐ, and
-Z-X-R₅ₐ;
Yₐ is selected from the group consisting of:
—O—,
—S(O)₀₋₂—,
—S(O)₂—N(R₈)—,
—N(R₈)-Q-,
—O—C(R₆)—N(R₈)—,
—C(R₆)—N(OR₉)—, 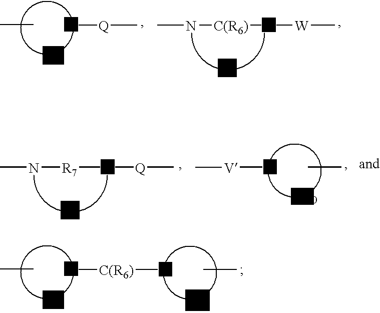
R₄ₐ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, trifluoromethyl, trifluoromethoxy, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, and (dialkylamino)alkyleneoxy; and
R₅ₐ is selected from the group consisting of:

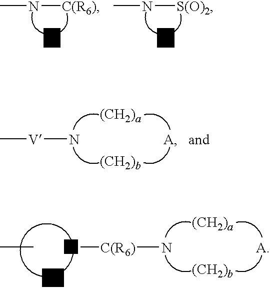

47. (canceled)

48. The method of claim 46 wherein R₂ₐ is selected from the group consisting of hydrogen, methyl, ethyl, propyl, butyl, 2-methoxyethyl, 2-hydroxyethyl, ethoxymethyl, and hydroxymethyl.

49.-52. (canceled)

53. The method of claim 46 wherein R₁ₐ is -X-Yₐ-R₄ₐ.

54. (canceled)

55. The method of claim 53 wherein -X-Yₐ-R₄ₐ is selected from the group consisting of 2-(propylsulfonyl)ethyl, 2-methyl-2-[(methylsulfonyl)amino]propyl, 4-methylsulfonylaminobutyl, and 2-(acetylamino)-2-methylpropyl.

56.-59. (canceled)

60. The method of claim 46 wherein R_{A1} and R_{B1} taken together form a fused benzene ring wherein the benzene ring is unsubstituted or substituted by one Rₐ group, or substituted by one R₃ₐ group, or substituted by one Rₐ group and one R₃ₐ group.

61.-62. (canceled)

63. The method of claim 39 wherein the fused benzene ring is unsubstituted.

64.-82. (canceled)

83. A compound of the Formula XI: 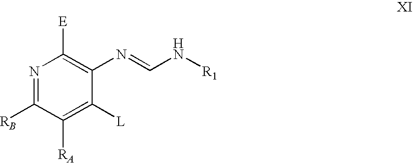
wherein:
E is selected from the group consisting of hydrogen, fluoro, chloro, bromo, iodo, hydroxy, phenoxy, —O—S(O)₂—R′, and —N(Bn)₂, wherein R′ is selected from the group consisting of alkyl, haloalkyl, and aryl optionally substituted by alkyl, halo, or nitro, and Bn is selected from the group consisting of benzyl, p-methoxybenzyl, p-methylbenzyl, and 2-furanylmethyl; or
E is joined with the adjacent pyridine nitrogen atom of Formula XI to form the fused tetrazolo ring in Formula XIII: 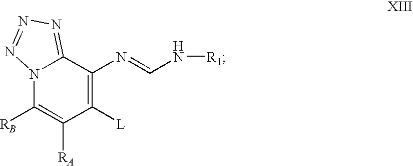
L is selected from the group consisting of fluoro, chloro, bromo, iodo, phenoxy, and —O—S(O)₂—R′, wherein R′ is selected from the group consisting of alkyl, haloalkyl, and aryl optionally substituted by alkyl, halo, or nitro;
R_{A} and R_{B} are independently selected from the group consisting of:
hydrogen,
halogen,
alkyl,
alkenyl,
alkoxy,
alkylthio, and
—N(R₉)₂;
or R_{A} and R_{B} taken together form a fused benzene ring or a fused pyridine ring wherein the benzene ring or pyridine ring is unsubstituted or substituted by one R group, or substituted by one R₃ group, or substituted by one R group and one R₃ group;
or R_{A} and R_{B} taken together form a fused 5 to 7 membered saturated ring optionally containing one nitrogen atom, wherein the fused ring is unsubstituted or substituted by one or more R groups;
R is selected from the group consisting of:
halogen,
hydroxy,
alkyl,
alkenyl,
haloalkyl,
alkoxy,
alkylthio, and
—N(R₉)₂;
R₁ is selected from the group consisting of:
-R₄,
-X-R₄,
-X-Y-R₄ₐ,
-X-Y-X-Y-R₄,
-X-R₅,
—N(R₁′)-Q-R₄,
—N(R₁′)—X₁—Y₁—R₄, and
—N(R₁′)—X₁—R_{5b};
R₃ is selected from the group consisting of:
-Z-R₄,
-Z-X-R₄,
-Z-X-Y-R₄,
-Z-X-Y-X-Y-R₄, and
-Z-X-R₅;
X is selected from the group consisting of alkylene, alkenylene, alkynylene, arylene, heteroarylene, and heterocyclylene wherein the alkylene, alkenylene, and alkynylene groups can be optionally interrupted or terminated by arylene, heteroarylene or heterocyclylene and optionally interrupted by one or more —O— groups;
X₁ is C₂₋₂₀ alkylene;
Y is selected from the group consisting of:
—O—,
—S(O)₀₋₂—,
—S(O)₂—N(R₈)—,
—C(R₆)—,
—O—C(R₆)—,
—O—C(O)—O—,
—N(R₈)-Q-,
—O—C(R₆)—N(R₉)—,
—C(R₆)—N(OR₉)—,
—O—N(R₈)-Q-,
—O—N═C(R₄)—,
—C(═N—O—R₈)—,
—CH(—N(—O—R₈)-Q-R₄)—, 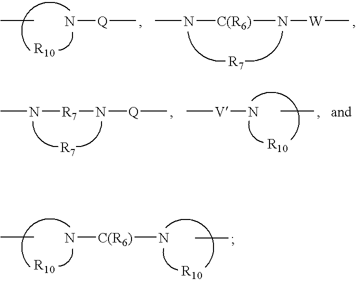
Y₁ is selected from the group consisting of —O—, —S(O)₀₋₂—, —S(O)₂—N(R₈)—, —N(R₈)-Q-, —C(R₆)—N(R₈)—, —O—C(R₆)—N(R₈)—, and 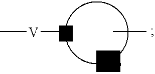
Z is a bond or —O—;
R₁′ is selected from the group consisting of hydrogen, C₁₋₂₀ alkyl, hydroxy-C₂₋₂₀ alkylenyl, and alkoxy-C₂₋₂₀ alkylenyl;
R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;
R₅ is selected from the group consisting of: 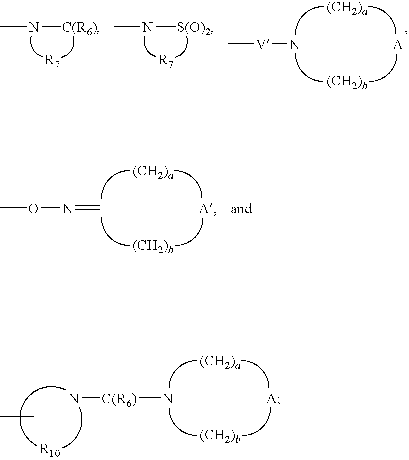
R_{5b} is selected from the group consisting of: 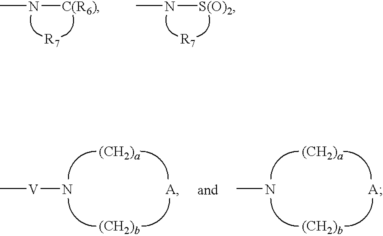
R₆ is selected from the group consisting of ═O and ═S;
R₇ is C₂₋₇ alkylene;
R₈ is selected from the group consisting of hydrogen, alkyl, alkoxyalkylenyl, hydroxyalkylenyl, arylalkylenyl, and heteroarylalkylenyl;
R₉ is selected from the group consisting of hydrogen and alkyl
R₁₀ is C₃₋₈ alkylene;
A is selected from the group consisting of —O—, —C(O)—, —S(O)₀₋₂—, and —N(R₄)—;
A′ is selected from the group consisting of —O—, —S(O)₀₋₂—, —N(-Q-R₄)—, and —CH₂—;
Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, —C(R₆)—S—, and —C(R₆)—N(OR₉)—;
V is selected from the group consisting of —C(R₆)—, —O—C(R₆)—, —N(R₆)—C(R₆)—, and —S(O)₂—;
V′ is selected from the group consisting of —O—C(R₆)—, —N(R₈)—C(R₆)—, and —S(O)₂—;
W is selected from the group consisting of a bond, —C(O)—, and —S(O)₂—; and
a and b are independently integers from 1 to 6 with the proviso that a+b is ≦7;
or a pharmaceutically acceptable salt thereof.

84.-86. (canceled)

87. The compound or salt of claim 83 wherein R₁ is -X-Y-R₄.

88. (canceled)

89. The compound or salt of claim 83 wherein -X-Y-R₄ is selected from the group consisting of 2-(propylsulfonyl)ethyl, 2-methyl-2-[(methylsulfonyl)amino]propyl, 4-methylsulfonylaminobutyl, and 2-(acetylamino)-2-methylpropyl.

90.-91. (canceled)

92. The compound or salt of claim 83 wherein R_{A} and R_{B} taken together form a fused benzene ring wherein the benzene ring is unsubstituted.

93. (canceled)

94. The method of claim 60 wherein the fused benzene ring is unsubstituted.
